SEQUENCE LISTING (1 sequence)

SEQ ID NO: 1 - protein, 8 aa, Artificial Sequence
  Synthetic construct
SIINFEKL

(19) United States
(12) Patent Application Publication
(10) Pub. No.: US 2023/0416746 A1
(43) Pub. Date: Dec. 28, 2023

(54) IMMUNO-ONCOLOGY TARGETS TO IMPROVE T-CELL METABOLIC RESPONSE

(71) Applicant: The Johns Hopkins University, Baltimore, MD (US)

(72) Inventors: Michael Lim, Reisterstown, MD (US); Christopher Jackson, Baltimore, MD (US); Ayush Pant, Baltimore, MD (US); Henry Brem, Ellicott City, MD (US)

(21) Appl. No.: 18/037,482

(22) PCT Filed: Nov. 18, 2021
(86) PCT No.: PCT/US2021/059900

§ 371 Date: May 17, 2023

Related U.S. Application Data
(60) Provisional application No. 63/115,803, filed on Nov. 19, 2020.
(60) Provisional application No. 63/246,146, filed on Sep. 20, 2021.

(51) Int. Cl. C12N 15/113; A61K 39/00; C07K 16/28; A61P 37/06

(52) U.S. Cl. CPC C12N 15/1136; A61K 39/4631; A61K 39/4611; C07K 16/2866; A61P 37/06; C12N 2310/14; C12N 2310/531; C12N 2310/11; C12N 2310/12

(57) ABSTRACT

The present invention relates to the field of cancer. More specifically, the present invention provides compositions and methods utilizing Meteorin-like (METRNL), C-X-C Motif Chemokine Receptor 6 (CXCR6) and/or endogenous C-X-C Motif Ligand 16 (CXCL16) as immune-oncology targets. Accordingly, in one aspect, the present invention provides compositions and methods directed to the knockout of METRNL, CXCR6 and/or CXCL16 expression in a cell. In particular embodiments, the cell is a T cell. In a specific embodiment, the present invention provides an engineered T-cell comprising disruption in the METRNL, CXCR6 and/or CXCL16 gene sequence. In another embodiment, an engineered T-cell comprises (a) at least one chimeric antigen receptor (CAR); and (b) at least one genomic disruption of METRNL, CXCR6 and/or CXCL16. In certain embodiments, the genomic disruption is performed using a CRSIPR endonuclease system.

### CROSS-REFERENCE TO RELATED APPLICATIONS

This application claims the benefit of U.S. Provisional Application No. 63/115,803, filed Nov. 19, 2020, and U.S. Provisional Application No. 63/246,146, filed Sep. 20, 2021, each of which is incorporated herein by reference in its entirety.

### FIELD OF THE INVENTION

The present invention relates to the field of cancer. More specifically, the present invention provides compositions and methods utilizing Meteorin-like (METRNL), C-X-C Motif Chemokine Receptor 6 (CXCR6) and/or endogenous C-X-C Motif Ligand 16 (CXCL16) as immune-oncology targets.

### BACKGROUND OF THE INVENTION

When immune cells infiltrate tumor tissue, they become inactive due to signals in the tumor environment. This process, known as exhaustion, is a major impediment to tumor immunotherapy and reversing exhaustion has proven to be a powerful strategy for treating solid tumors. The majority of tumors, however, fail to respond to immunotherapy, indicating a need for novel therapies and combination therapies.

### SUMMARY OF THE INVENTION

The present inventors used RNA sequencing of tumor-infiltrating lymphocytes (TIL) and matched lymphocytes from the peripheral blood (PBL) of patients with kidney cancer, bladder cancer, prostate cancer, and glioblastoma to identify genes specifically unregulated in exhausted TIL across tumor types. This comparative transcriptomics approach identified two genes, METRNL and CXCR6 that were unregulated in TIL compared with PBL in all tumor types and also were associated with expression of immune checkpoints (an indicator of exhaustion). The present inventors then verified that tumor-infiltrating lymphocytes express METRNL protein and exogenous Metrnl inhibits immune cell function in vitro. In addition, the present inventors verified that CXCR6 is a key mediator of T-cell exhaustion and have identified metabolic alterations in TILs elicited by CXCR6 signaling which lead to amelioration of T-cell function. Taken together, these results identify METRNL and CXCR6 as novel mediators of metabolic exhaustion of T-cells across many tumor types.

The discovery of METRNL/CXCR6 promoting mitochondrial exhaustion of T-cells can be used, in particular embodiments, to prolong survival and activity of endogenous anti-tumor T-cells or of CAR-T-cell therapy. In specific embodiments, a therapeutic blocking METRNL, CXCR6 and/or its ligand CXCL16, including but not limited to, an antibody, may have potential as an immune-oncology target across a variety of solid tumors.

Accordingly, in one aspect, the present invention provides compositions and methods directed to the knockout of METRNL, CXCR6 and/or CXCL16 expression in a cell. In particular embodiments, the cell is a T cell. In a specific embodiment, the present invention provides an engineered T-cell comprising a disruption in an endogenous METRNL, CXCR6 and/or CXCL16 gene sequence. In another embodiment, an engineered T-cell comprises (a) at least one chimeric antigen receptor (CAR); and (b) at least one genomic disruption of METRNL, CXCR6 and/or CXCL16. In certain embodiments, the genomic disruption is performed using a CRSIPR endonuclease system.

In another aspect, the present invention provides compositions and methods directed to the treatment of cancer. In one embodiment, a method of treating cancer in a patient comprising the step of administering to the patient an effective amount of an engineered T cell described herein. In another embodiment, a method for treating cancer in a patient comprises the step of administering to the patient an effective amount of a METRNL, CXCR6 and/or CXCL16 inhibitor. In certain embodiments, the METRNL, CXCR6 and/or CXCL16 inhibitor is selected from the group consisting of a small molecule, a polypeptide, a nucleic acid molecule, a peptidomimetic, or a combination thereof. In a specific embodiment, the agent can be a polypeptide. The polypeptide can, for example, comprise an antibody. In another embodiment, the agent can be a nucleic acid molecule. The nucleic acid molecule can, for example, be a METRNL, CXCR6 or CXCL16 inhibitory nucleic acid molecule. The METRNL, CXCR6 or CXCL16 inhibitory nucleic acid molecule can comprise a short interfering RNA (siRNA) molecule, a microRNA (miRNA) molecule, or an antisense molecule.

In a further aspect, the present invention provides compositions and methods directed to the treatment of autoimmune diseases. In one embodiment, a method for treating an autoimmune disorder in a patient comprises the step of administering to the patient an effective amount of a METRNL, CXCR6 and/or CXCL16 agonist. In another embodiment, the patient is administered an effective amount of METRNL, CXCR6 and/or CXCL16 protein or a functional part thereof.

In yet another aspect, the compositions and methods can be used to screen for modulators of METRNL, CXCR6 and/or CXCL16. The assay can be used to identify agonists or antagonists of METRNL, CXCR6 and/or CXCL16. In certain embodiments, a method comprises the steps of (a) contacting a cell with a test agent; and (c) measuring the amount of METRNL, CXCR6 and/or CXCL16 using at least one anti-METRNL, anti-CXCR6 and/or anti-CXCL16 antibody or antigen-binding fragment thereof. In another embodiment, a method of identifying a modulator of METRNL, CXCR6 and/or CXCL16 comprises the steps of (a) contacting cells with a test agent; and (b) detecting a change in the amount of METRNL, CXCR6 and/or CXCL16 in the cell as compared to the amount of METRNL, CXCR6 and/or CXCL16 in a cell not contacted with the test agent. In certain embodiments, the detecting step utilizes at least one anti-METRNL, anti-CXCR6 and/or anti-CXCL16 antibody or antigen-binding fragment thereof.

### BRIEF DESCRIPTION OF THE FIGURES

FIG. 1A-1C. CD8 gene expression patterns cluster by location. Differential expression analysis of experienced CD8 TIL samples and activated CD8 PBL samples for GBM, PRAD, RCC and BLCA cohorts. (FIG. A) After FDR adjustments, genes that meet an FDR<=0.05 are highlighted. Genes that meet an FDR<=0.00001 are labeled. Red indicates higher expression in the TIL samples and blue indicates lower expression compared with PBL. (FIG. B) GSEA pathways analysis for differentially expressed genes based on the Biological Hallmarks dataset. Gene sets were selected if FDR<0.01 in one of the tumor types. The color scale is log 10(FDR), with orange indicating higher expression in CD8 TILs vs. activated PBLs and blue indicating higher expression in activated PBLs vs. CD8 TILs. (FIG. C) Estimating the underlying high expression and low expression distributions via expectation maximization. Cutpoints between the two distributions were calculated to obtain a gene expression level cutoff.

FIG. 2A-2D: Differential expression of METRNL and CXCR6 is associated with intratumoral location and immune checkpoint expression. Comparison between tissue cohorts. (FIG. 2A) Venn diagram displaying significantly differentially expressed (FDR<0.01) genes across tissues for triple positive experienced CD8 tumor samples contrasted against all other samples. (FIG. 2B) Venn diagram displaying significantly differentially expressed (FDR<0.0001) genes across tissues for triple positive experienced CD8 tumor samples contrasted against triple positive activated PBL CD8 samples. (FIG. 2C) Statistics for METRNL in the triple positive vs all other samples analysis. (FIG. 2D) Statistics for METRNL in the triple positive TIL vs triple positive PBL analysis.

FIG. 3A-3E: Metrnl is an immunosuppressive cytokine present in glioblastoma tissue and associated with checkpoint expression. (FIG. 3A) Metrnl is secreted by immune checkpoint expressing CD8 T cells isolated from murine GL261 gliomas and immune checkpoints are associated with decreased IFN-g secretion. (FIG. 3B) Scatter plot showing Metrnl and IFNg concentrations by well. (FIG. 3C) Exogenous Metrnl Inhibits IFN-g secretion by CD8 T cells in the presence of cognate antigen in a dose-dependent manner. These experiments were repeated twice with consistent results. (FIG. 3D, 3E) Injection of Metrnl-hydrogel admixture at the site of MC38 flank tumors accelerates growth of the tumor. This experiment was performed >3 times with comparable results. Graphs show the mean+/−SEM. Statistics were calculated by one-way ANOVA with Tukey's multiple-comparisons post hoc test (FIG. 3B, 3D). Pearson's correlation (FIG. 3C), 2-way ANOVA with Sidak's multiple-comparisons test (FIG. 3E) and two-tailed unpaired Student's t test. *p≤0.05; **p≤0.01; ***p≤0.001; ****p≤0.0001; ns, not significant.

FIG. 4A-4E: Metrnl KO mice exhibit delayed tumor growth and enhanced CD8 TIL effector function and viability. Survival of Metrnl KO and WT mice with (FIG. 4A) orthotopic GL261 glioma, (FIG. 4B) B6CaP prostate cancer flank tumors, (FIG. 4C) MC38 colorectal cancer flank tumors. (FIG. 4D) Anti-CD8 depletion abrogated the growth suppression of MC38 in METRNL KO mice. (FIG. 4E) Flow cytometry analysis of TILs from MC38 flank tumors show no difference in checkpoint expression with Metrnl KO but do show an increase in IFN-g expression and a decrease in numbers of apoptotic cells. Differences in survival were calculated by the Mantel-Cox log-rank test. Graphs show the mean+/−SEM. Statistics for tumor volumes were calculated with 2-way ANOVA with Sidak's multiple-comparisons test. Statistics for immune checkpoints and markers of activation and apoptosis were calculated using two-tailed unpaired Student's t test. *p≤0.05; **p≤0.01; ****p≤0.0001; ns, not significant.

FIG. 5A-5E: Metrnl depolarizes mitochondria in CD8 T cells. Flow cytometry analysis of CD8 T cells treated with increasing doses of exogenous Metrnl during activation with PMA/ionomycin shows (FIG. 5A) an inverse correlation with retention of potential-dependent dye Mitotracker Red CMXRos, (FIG. 5B) and uniform staining with potential-independent dye Mitotracker Green. (FIG. 5C) TMRM staining of CD8 T cells assumes a punctate pattern of dye localization in untreated cells, whereas a diffuse, solid staining pattern is observed in Metrnl-treated cells. (FIG. 5D) Three to four images were quantified for each condition and the experiment was repeated three times with consistent results. (FIG. 5E) Injection of exogenous Metrnl at the MC38 flank tumor site decreases the percentage of CD8 TILs with polarized mitochondria and increases the percentage of TILs with depolarized mitochondria, as indicated by co-staining with potential-dependent Mitotracker Deep Red dye and potential-independent Mitotracker Green. Graphs show post hoc test (FIG. 5A, 5B, 5C), 2-way ANOVA with Holm-Sidak correction (FIG. 5E) and two-tailed unpaired Student's t test. *p≤0.05; **p≤0.01; ****p≤0.0001; ns, not significant.

FIG. 6A-6D: Metml increases ROS accumulation and apoptosis. (FIG. 6A) Flow cytometry analysis of CD8 T cells treated with increasing doses of exogenous Metrnl during activation of PMA/ionomycin showing increasing retention of ROS-staining dye Mitosox. (FIG. 6B) Representative images of Apopxin and Hoescht staining of activated CD8 T cells with and without exogenous Metml. (FIG. 6C) For each treatment three to four images were quantified, and the experiment was repeated four times with consistent results. (FIG. 6D) Flow cytometry analysis of Annexin V and Propidium Iodide co-staining of TILs isolated from MC38 flank tumors shows a decrease in apoptotic cells in Metml KO compared with WT. Graphs show the mean+/−SEM. Statistics were calculated with one-way ANOVA with Tukey's multiple-comparisons post hoc test (FIG. 6A), 2-way ANOVA with Holm-Sidak correction (FIG. 6C), and two-tailed unpaired Student's t test (FIG. 6D). *p≤0.05; **p≤0.01; ***p≤0.001; ns, not significant.

FIG. 7A-7D: Metrnl alters CD8 T cell metabolism, increasing oxidative stress and a triggering a compensatory anti-oxidative stress response. (FIG. 7A) Heatmap visualization of the top 25 metabolite changes between untreated and Metml-treated CD8 T cells, measured by LC-MS. (FIG. 7B) Relative amounts of metabolites related too glycolytic flux, pentose phosphate pathway, and oxidative stress response in untreated and Metml-treated CD8 T cells. (FIG. 7C) Volcano plot of metabolites plotting log 2 fold change versus −log 10 (FRD-corrected p value), with red/blue representing significant metabolite changes. (FIG. 7D) Impaired glucose uptake in Metml-treated CD8 T cells during activation, as measured by incorporation of glucose analogue 2-NBDG shows downregulation of glycolytic flux from LC-MS analysis. Graphs show mean+/−SD (FIG. 7B) and mean+/−SEM (FIG. 7D). Statistical significance was analyzed by unpaired two-tailed Student's t test (FIG. 7A, 7C) and one-way ANOVA with Tukey's multiple-comparisons post hoc test (FIG. 7D). *p≤0.1; **p≤0.01; ***p≤0.001 by t test (FIG. 7A, 7C). *p≤0.05; **p≤0.01; ***p≤0.001 (FIG. 7B, 7D).

FIG. 8A-8D. Ranges of expression for known genes of interest in (FIG. 8A) GBM, (FIG. 8B) PRAD, (FIG. 8C) RCC, and (FIG. 8D) BLCA cohorts. Boxplots of gene expression for known genes of interest in relation to immune checkpoints for glioblastoma data. Many of the genes demonstrate expected behavior with respect to relative range of expression between cell types.

FIG. 9A-9B: Comparison of samples by expression of immune checkpoints for GBM, PRAD, RCC and BLCA cohorts. (FIG. 9A) Gene expression values are plotted against each other for pairs of genes. Each label next to a point is a patient identifier. In multiple plots, the activated PBMC samples have higher expression for both markers compared to the patient-matched naive PBMC sample. (FIG. 9B) The range of distribution for each immune checkpoint indicates an underlying bimodal distribution.

FIG. 10. Confirming expression of CXCR6 on TILs.

FIG. 11. CXCR6 is upregulated in GBM in response to immunotherapy, acts as alternative checkpoint.

FIG. 12. CXCR6 is upregulated in GBM in response to many immunotherapy combinations.

FIG. 13. CXCR6 is upregulated in exhausted T-cells.

FIG. 14. CXCR upregulation indicates activation status, but CXCL16:CXCR6 signaling dampens activation.

FIG. 15. Role of CXCR6 activation on mitochondrial health.

FIG. 16. CXCL16 stimulation increases reactive oxygen species in T-cells.

FIG. 17. Sustained activation of CXCR6 in tumor increases tumor growth. CXCL16→CXCR6 signaling at flank tumor site (B16F10 melanoma) exacerbates tumor growth.

FIG. 18. Systemically injected Metrnl siRNA slows tumor progression of flank MC38 tumors. 100,000 MC38 cells were injected in the right flank of C57BL/6 mice. On day 10, 5 ug of siRNA against Metrnl and scrambled siRNA contained in nanoparticles were administered via retro-orbital injection. Tumor measurements were taken 2-3 times a week using calipers.

FIG. 19. CXCL16, the ligand that activates CXCR6, knockdown at the tumor site. Intratumoral injection of CXCL16 siRNA slows tumor progression of flank B16F10 tumors. 100,000 B16F10 cells were injected in the right flank of C57BL/6 mice. On day 10 and day 15, 10 ug of siRNA contained in nanoparticles were mixed with hydrogel in a 1:1 ratio by volume and injected at the tumor site. Tumor measurements were taken 2-3 times a week using calipers.

FIG. 20. METRNL adoptive cell therapy treatment. 1×10¹⁶ B16-Ova cells were injected in the right flank of C57BL/6 recipient mice. On day 6, 1×10⁶ T cells from OT-1^{metnl KO} donor mice that had been stimulated with anti-CD3/anti-CD28 beads for 7 days in vitro were injected in recipients with palpable tumors via the tail vein. Tumor volumes were measured using calipers twice a week.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

As used herein, the articles “a” and “an” are used to refer to one or to more than one (i.e., to at least one) of the grammatical object of the article. By way of example, “an element” means one element or more than one element.

As used herein, “about,” when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. Unless specifically stated or obvious from context, as used herein, the term “about” is understood as within a range of normal tolerance in the art, for example within 2 standard deviations of the mean. About can be understood as being within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term “about.”

Optional” or “optionally” means that the subsequently described event or circumstance can or cannot occur, and that the description includes instances where the event or circumstance occurs and instances where it does not.

As used herein, the term “modulate” indicates the ability to control or influence directly or indirectly, and by way of non-limiting examples, can alternatively mean inhibit or stimulate, agonize or antagonize, hinder or promote, and strengthen or weaken. Thus, the term “METRNL modulator” refers to an agent that modulates the expression and/or activity of METRNL. Similarly, the terms “CXCR6 modulator” and “CXCL16 modulator” refers to an agent that modulates the expression and/or activity of CXCR6 and CXCL16, respectively. Inhibitors may be organic or inorganic, small to large molecular weight individual compounds, mixtures and combinatorial libraries of inhibitors, agonists, antagonists, and biopolymers such as peptides, nucleic acids, or oligonucleotides. A modulator may be a natural product or a naturally-occurring small molecule organic compound. In particular, a modulator may be a carbohydrate; monosaccharide; oligosaccharide; polysaccharide; amino acid; peptide; oligopeptide; polypeptide; protein; receptor; nucleic acid; nucleoside; nucleotide; oligonucleotide; polynucleotide including DNA and DNA fragments, RNA and RNA fragments and the like; lipid; retinoid; steroid; glycopeptides; glycoprotein; proteoglycan and the like; and synthetic analogues or derivatives thereof, including peptidomimetics, small molecule organic compounds and the like, and mixtures thereof. A modulator identified according to the invention is preferably useful in the treatment of a disease disclosed herein.

An “agonist” is a type of modulator and refers to an agent that can activate one or more functions of the target. For example, an agonist of a protein can activate the protein in the absence of its natural or cognate ligand.

As used herein, an “antagonist” is a type of modulator and is used interchangeably with the term “inhibitor.” In certain non-limiting embodiments, the term refers to an agent that can inhibit a one or more functions of the target. For example, an antagonist of an enzymatic protein can inhibit the enzymatic activity of the protein.

The term “inhibitor” is a type of modulator and is used interchangeably with the term “antagonist.” The term “inhibitor” includes any type of molecule or agent that directly or indirectly inhibits the expression or activity of a target gene or protein. An inhibitor can be any type of compound, such as a small molecule, polypeptide, polynucleotide and the like including an antibody or an RNA interference compound. In certain embodiments, the target gene or protein is METRNL. The term also includes agents that have activity in addition to METRNL inhibitory activity. In particular embodiments, the target gene or protein is CXCR6. The term also includes agents that have activity in addition to CXCR6 inhibitory activity. CXCL16 inhibitors are also contemplated herein. In particular embodiments, the target gene or protein in CXCL16.

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules. In addition, as used herein, the term “polypeptide” refers to amino acids joined to each other by peptide bonds or modified peptide bonds, e.g., peptide isosteres, etc., and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. See Proteins-Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983).

By “probe,” “primer,” or oligonucleotide is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for METRNL, CXCR6 or CXCL16 nucleic acids (for example, genes and/or mRNAs) have at least 80%-90% sequence complementarity, preferably at least 91%-95% sequence complementarity, more preferably at least 96%-99% sequence complementarity, and most preferably 100% sequence complementarity to the region of the METRNL, CXCR6 or CXCL16 nucleic acid to which they hybridize. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

The term “antibody” means an immunoglobulin molecule that recognizes and specifically binds to a target, such as a protein (e.g., METRNL, CXCR6 or CXCL16), polypeptide, peptide, carbohydrate, polynucleotide, lipid, or combinations of the foregoing through at least one antigen recognition site within the variable region of the immunoglobulin molecule. A typical antibody comprises at least two heavy (HC) chains and two light (LC) chains interconnected by disulfide bonds. Each heavy chain is comprised of a “heavy chain variable region” or “heavy chain variable domain” (abbreviated herein as VH) and a heavy chain constant region (CH). The heavy chain constant region is comprised of three domains, CH1, CH2, and CH3. Each light chain is comprised of a “light chain variable region” or “light chain variable domain” (abbreviated herein as VL) and a light chain constant region. The light chain constant region is comprised of one domain, CL. The VH and VL regions can be further subdivided into regions of hypervariablity, termed Complementarity Determining Regions (CDR), interspersed with regions that are more conserved, termed framework regions (FRs). Each VH and VL region is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. As used herein, the term “antibody” encompasses intact polyclonal antibodies, intact monoclonal antibodies, antibody fragments (such as Fab, Fab′, F(ab′)2, Fd, Facb, and Fv fragments), single chain Fv (scFv), minibodies (e.g., sc(Fv)2, diabody), multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. Thus, the term “antibody” includes whole antibodies and any antigen-binding fragment or single chains thereof. Antibodies can be naked or conjugated to other molecules such as toxins, detectable labels, radioisotopes, small molecule drugs, polypeptides, etc.

The term “isolated antibody” refers to an antibody that has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibody is purified (1) to greater than 95% by weight of antibody as determined by, for example, the Lowry method, and including more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or silver stain. An isolated antibody includes the antibody in situ within recombinant cells since at least one component of the antibody's natural environment will not be present. Ordinarily, however, isolated antibody will be prepared by at least one purification step.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, “substantially purified” refers to being essentially free of other components. For example, a substantially purified polypeptide is a polypeptide which has been separated from other components with which it is normally associated in its naturally occurring state.

The term “humanized” immunoglobulin refers to an immunoglobulin comprising a human framework region and one or more CDRs from a non-human (usually a mouse or rat) immunoglobulin. The non-human immunoglobulin providing the CDRs is called the “donor” and the human immunoglobulin providing the framework is called the “acceptor.” Constant regions need not be present, but if they are, they must be substantially identical to human immunoglobulin constant regions, i.e., at least about 85-90%, preferably about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. For example, a humanized antibody would not encompass a typical chimeric antibody as defined herein, e.g., because the entire variable region of a chimeric antibody is non-human.

The term “antigen” is generally used in reference to any substance that is capable of reacting with an antibody. An antigen can also refer to a synthetic peptide, polypeptide, protein or fragment of a polypeptide or protein, or other molecule which elicits an antibody response in a subject, or is recognized and bound by an antibody. The term can refer to a molecule that contains one or more epitopes capable of being bound by one or more receptors. For example, an antigen can stimulate a host's immune system to make a cellular antigen-specific immune response when the antigen is presented, or a humoral antibody response. An antigen can also have the ability to elicit a cellular and/or humoral response by itself or when present in combination with another molecule. For example, a tumor cell antigen can be recognized by a T-cell receptor (TCR).

The term “antigen-binding fragment” refers to a portion of an intact antibody and refers to the antigenic determining variable regions of an intact antibody. It is known in the art that the antigen-binding function of an antibody can be performed by fragments of a full-length antibody. Examples of antigen-binding antibody fragments include, but are not limited to Fab, Fab′, F(ab′)2, Facb, Fd, and Fv fragments, linear antibodies, single chain antibodies, and multi-specific antibodies formed from antibody fragments. In some instances, antibody fragments may be prepared by proteolytic digestion of intact or whole antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab′)2 or Fab′; and plasmin digestion of whole antibodies yields Facb fragments.

The term “Fab” refers to an antibody fragment that is essentially equivalent to that obtained by digestion of immunoglobulin (typically IgG) with the enzyme papain. The heavy chain segment of the Fab fragment is the Fd piece. Such fragments can be enzymatically or chemically produced by fragmentation of an intact antibody, recombinantly produced from a gene encoding the partial antibody sequence, or it can be wholly or partially synthetically produced. The term “F(ab′)2” refers to an antibody fragment that is essentially equivalent to a fragment obtained by digestion of an immunoglobulin (typically IgG) with the enzyme pepsin at pH 4.0-4.5. Such fragments can be enzymatically or chemically produced by fragmentation of an intact antibody, recombinantly produced from a gene encoding the partial antibody sequence, or it can be wholly or partially synthetically produced. The term “Fv” refers to an antibody fragment that consists of one NH and one N domain held together by noncovalent interactions.

The terms “METRNL antibody,” “anti-METRNL antibody,” “anti-METRNL,” “antibody that binds to METRNL” and any grammatical variations thereof refer to an antibody that is capable of specifically binding to METRNL with sufficient affinity such that the antibody could be useful, for example, as a therapeutic agent or diagnostic reagent in targeting METRNL. The extent of binding of an anti-METRNL antibody disclosed herein to an unrelated, non-METRNL protein is less than about 10% of the binding of the antibody to METRNL as measured, e.g., by a radioimmunoassay (RIA), BIACORE™ (using recombinant METRNL as the analyte and antibody as the ligand, or vice versa), or other binding assays known in the art. In certain embodiments, an antibody that binds to METRNL has a dissociation constant (1(D) of <1 μM, <100 nM, <50 nM, <10 nM, or <1 nM.

The terms “CXCR6 antibody,” “anti-CXCR6 antibody,” “anti-CXCR6,” “antibody that binds to CXCR6” and any grammatical variations thereof refer to an antibody that is capable of specifically binding to CXCR6 with sufficient affinity such that the antibody could be useful, for example, as a therapeutic agent or diagnostic reagent in targeting CXCR6. The extent of binding of an anti-CXCR6 antibody disclosed herein to an unrelated, non-CXCR6 protein is less than about 10% of the binding of the antibody to CXCR6 as measured, e.g., by a radioimmunoassay (RIA), BIACORE™ (using recombinant CXCR6 as the analyte and antibody as the ligand, or vice versa), or other binding assays known in the art. In certain embodiments, an antibody that binds to CXCR6 has a dissociation constant (KD) of <1 μM, <100 nM, <50 nM, <10 nM, or <1 nM.

The terms “CXCL16 antibody,” “anti-CXCL16 antibody,” “anti-CXCL16,” “antibody that binds to CXCL16” and any grammatical variations thereof refer to an antibody that is capable of specifically binding to CXCL16 with sufficient affinity such that the antibody could be useful, for example, as a therapeutic agent or diagnostic reagent in targeting CXCL16. The extent of binding of an anti-CXCL16 antibody disclosed herein to an unrelated, non-CXCL16 protein is less than about 10% of the binding of the antibody to CXCL16 as measured, e.g., by a radioimmunoassay (RIA), BIACORE™ (using recombinant CXCL16 as the analyte and antibody as the ligand, or vice versa), or other binding assays known in the art. In certain embodiments, an antibody that binds to CXCL16 has a dissociation constant (KD) of <1 μM, <100 nM, <50 nM, <10 nM, or <1 nM. In particular embodiments, an anti-CXCL16 antibody prevents CXCL16 from binding its receptor CXCR6.

The term “% identical” (“sequence identity”) between two polypeptide (or polynucleotide) sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site. Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence. One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2 (ClustalX is a version of the ClustalW2 program ported to the Windows environment). Another suitable program is MUSCLE. ClustalW2 and MUSCLE are alternatively available, e.g., from the European Bioinformatics Institute (EBI).

By “detectable label” is meant a composition that when linked (directly or indirectly) to a molecule of interest renders the latter detectable via spectroscopic, photochemical, biochemical, immunochemical, chemical or electrochemiluminescent means. For example, detectable labels include radioactive isotopes, magnetic beads, metallic beads, colloidal particles, fluorescent dyes, electron-dense reagents, enzymes (for example, as commonly used in an ELISA), biotin, digoxigenin, or haptens. The labeling of an antigen can be carried out by any generally known method. Examples of the detectable label known to those skilled in the art include a fluorescent dye, an enzyme, a coenzyme, a chemiluminescent substance or a radioactive substance. Specific examples may include radioisotopes (³²P, ¹⁴C, ¹²⁵I, ³H, ¹³¹I and the like), fluorescein, rhodamine, dansyl chloride, umbelliferone, luciferase, peroxidase, alkaline phosphatase, beta-galactosidase, beta-glucosidase, horseradish peroxidase, glucoamylase, lysozyme, saccharide oxidase, microperoxidase, biotin and the like.

The term “epitope” and its grammatical equivalents as used herein can refer to a part of an antigen that can be recognized by antibodies, B cells, T cells or engineered cells. For example, an epitope can be a cancer epitope that is recognized by a T cell receptor (TCR). Multiple epitopes within an antigen can also be recognized. The epitope can also be mutated.

The term “autologous” and its grammatical equivalents as used herein can refer to as originating from the same being. For example, a sample (e.g., cells) can be removed, processed, and given back to the same subject (e.g., patient) at a later time. An autologous process is distinguished from an allogenic process where the donor and the recipient are different subjects.

The term “cancer” and its grammatical equivalents as used herein can refer to a hyperproliferation of cells whose unique trait—loss of normal controls—results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, rectal cancer, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin's lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and/or urinary bladder cancer. As used herein, the term “tumor” refers to an abnormal growth of cells or tissues, e.g., of malignant type or benign type.

The term “cancer neo-antigen” or “neo-antigen” or “neo-epitope” and its grammatical equivalents as used herein can refer to antigens that are not encoded in a normal, non-mutated host genome. A “neo-antigen” can in some instances represent either oncogenic viral proteins or abnormal proteins that arise as a consequence of somatic mutations. For example, a neo-antigen can arise by the disruption of cellular mechanisms through the activity of viral proteins. Another example can be an exposure of a carcinogenic compound, which in some cases can lead to a somatic mutation. This somatic mutation can ultimately lead to the formation of a tumor/cancer.

The term “cytotoxicity” as used in this specification, refers to an unintended or undesirable alteration in the normal state of a cell. The normal state of a cell may refer to a state that is manifested or exists prior to the cell's exposure to a cytotoxic composition, agent and/or condition. Generally, a cell that is in a normal state is one that is in homeostasis. An unintended or undesirable alteration in the normal state of a cell can be manifested in the form of, for example, cell death (e.g., programmed cell death), a decrease in replicative potential, a decrease in cellular integrity such as membrane integrity, a decrease in metabolic activity, a decrease in developmental capability, or any of the cytotoxic effects disclosed in the present application.

The term “engineered” and its grammatical equivalents as used herein can refer to one or more alterations of a nucleic acid, e.g., the nucleic acid within an organism's genome. The term “engineered” can refer to alterations, additions, and/or deletion of genes. An engineered cell can also refer to a cell with an added, deleted and/or altered gene.

The term “cell” or “engineered cell” and their grammatical equivalents as used herein can refer to a cell of human or non-human animal origin.

A “CRISPR,” “CRISPR system,” or “CRISPR nuclease system” and their grammatical equivalents can include a non-coding RNA molecule (e.g., guide RNA) that binds to DNA and Cas proteins (e.g., Cas9) with nuclease functionality (e.g., two nuclease domains). See, e.g., Sander, J. D., et al., “CRISPR-Cas systems for editing, regulating and targeting genomes,” Nature Biotechnology, 32:347-355 (2014); see also e.g., Hsu, P. D., et al., “Development and applications of CRISPR-Cas9 for genome engineering,” Cell 157(6):1262-1278 (2014).

The term “disrupting” and its grammatical equivalents as used herein can refer to a process of altering a gene, e.g., by deletion, insertion, mutation, rearrangement, or any combination thereof. For example, a gene can be disrupted by knockout. Disrupting a gene can be partially reducing or completely suppressing expression of the gene. Disrupting a gene can also cause activation of a different gene, for example, a downstream gene.

The term “gene editing” and its grammatical equivalents as used herein can refer to genetic engineering in which one or more nucleotides are inserted, replaced, or removed from a genome. Gene editing can be performed using a nuclease (e.g., a natural-existing nuclease or an artificially engineered nuclease).

The term “mutation” and its grammatical equivalents as used herein can include the substitution, deletion, and insertion of one or more nucleotides in a polynucleotide. For example, up to 1, 2, 3, 4, 5, 6, 7, 8, 9, 10, 11, 12, 13, 14, 15, 20, 25, 30, 40, 50, or more nucleotides/amino acids in a polynucleotide (cDNA, gene) or a polypeptide sequence can be substituted, deleted, and/or inserted. A mutation can affect the coding sequence of a gene or its regulatory sequence. A mutation can also affect the structure of the genomic sequence or the structure/stability of the encoded mRNA.

The term “hon-human animal” and its grammatical equivalents as used herein can include all animal species other than humans, including non-human mammals, which can be a native animal or a genetically modified non-human animal.

The terms “nucleic acid,” “polynucleotide,” “polynucleic acid,” and “oligonucleotide” and their grammatical equivalents can be used interchangeably and can refer to a deoxyribonucleotide or ribonucleotide polymer, in linear or circular conformation, and in either single- or double-stranded form. For the purposes of the present disclosure, these terms should not be construed as limiting with respect to length. The terms can also encompass analogues of natural nucleotides, as well as nucleotides that are modified in the base, sugar and/or phosphate moieties (e.g., phosphorothioate backbones). Modifications of the terms can also encompass demethylation, addition of CpG methylation, removal of bacterial methylation, and/or addition of mammalian methylation. In general, an analogue of a particular nucleotide can have the same base-pairing specificity, i.e., an analogue of A can base-pair with T.

The term “peripheral blood lymphocytes” (PBL) and its grammatical equivalents as used herein can refer to lymphocytes that circulate in the blood (e.g., peripheral blood). Peripheral blood lymphocytes can refer to lymphocytes that are not localized to organs. Peripheral blood lymphocytes can comprise T cells, NK cells, B cell, or any combinations thereof.

The term “recipient” and their grammatical equivalents as used herein can refer to a human or non-human animal. The recipient can also be in need thereof.

The term “recombination” and its grammatical equivalents as used herein can refer to a process of exchange of genetic information between two polynucleic acids. For the purposes of this disclosure, “homologous recombination” or “HR” can refer to a specialized form of such genetic exchange that can take place, for example, during repair of double-strand breaks. This process can require nucleotide sequence homology, for example, using a donor molecule to template repair of a target molecule (e.g., a molecule that experienced the double-strand break), and is sometimes known as non-crossover gene conversion or short tract gene conversion. Such transfer can also involve mismatch correction of heteroduplex DNA that forms between the broken target and the donor, and/or synthesis-dependent strand annealing, in which the donor can be used to resynthesize genetic information that can become part of the target, and/or related processes. Such specialized HR can often result in an alteration of the sequence of the target molecule such that part or all of the sequence of the donor polynucleotide can be incorporated into the target polynucleotide. In some cases, the terms “recombination arms” and “homology arms” can be used interchangeably.

The terms “target vector” and “targeting vector” are used interchangeably herein.

The term “transgene” and its grammatical equivalents as used herein can refer to a gene or genetic material that is transferred into an organism. For example, a transgene can be a stretch or segment of DNA containing a gene that is introduced into an organism. When a transgene is transferred into an organism, the organism is then referred to as a transgenic organism. A transgene can retain its ability to produce RNA or polypeptides (e.g., proteins) in a transgenic organism. A transgene can be composed of different nucleic acids, for example RNA or DNA. A transgene may encode for an engineered T cell receptor, for example a TCR transgene. A transgene may comprise a TCR sequence. A transgene can comprise recombination arms. A transgene can comprise engineered sites.

The term “T cell” and its grammatical equivalents as used herein can refer to a T cell from any origin. For example, a T cell can be a primary T cell, e.g., an autologous T cell, a cell line, etc. The T cell can also be human or non-human.

The term “TIL” or tumor infiltrating lymphocyte and its grammatical equivalents as used herein can refer to a cell isolated from a tumor. For example, a TIL can be a cell that has migrated to a tumor. A TIL can also be a cell that has infiltrated a tumor. A TIL can be any cell found within a tumor. For example, a TIL can be a T cell, B cell, monocyte, natural killer cell, or any combination thereof. A TIL can be a mixed population of cells. A population of TILs can comprise cells of different phenotypes, cells of different degrees of differentiation, cells of different lineages, or any combination thereof.

A “therapeutic effect” may occur if there is a change in the condition being treated. The change may be positive or negative. For example, a ‘positive effect’ may correspond to an increase in the number of activated T-cells in a subject. In another example, a ‘negative effect’ may correspond to a decrease in the amount or size of a tumor in a subject. There is a “change” in the condition being treated if there is at least 10% improvement, preferably at least 25%, more preferably at least 50%, even more preferably at least 75%, and most preferably 100%. The change can be based on improvements in the severity of the treated condition in an individual, or on a difference in the frequency of improved conditions in populations of individuals with and without treatment with the therapeutic compositions with which the compositions of the present invention are administered in combination. Similarly, a method of the present disclosure may comprise administering to a subject an amount of cells that is “therapeutically effective”. The term “therapeutically effective” should be understood to have a definition corresponding to ‘having a therapeutic effect’.

The term “sequence” and its grammatical equivalents as used herein can refer to a nucleotide sequence, which can be DNA or RNA; can be linear, circular or branched; and can be either single-stranded or double stranded. A sequence can be mutated. A sequence can be of any length, for example, between 2 and 1,000,000 or more nucleotides in length (or any integer value there between or there above), e.g., between about 100 and about 10,000 nucleotides or between about 200 and about 500 nucleotides.

The term “autoimmune disease” including ankylosing spondylitis, chronic inflammatory demyelinating polyneuropathy (CIDP), Crohn's disease, dermatomyositis, Graves' disease, Guillain-Barre syndrome, lupus, multiple sclerosis, myasthenia gravis, polyarteritis nodosa, primary biliary cirrhosis, psoriatic arthritis, rheumatoid arthritis, scleroderma and ulcerative colitis. The term further includes, but is not limited to, achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease, autoimmune myocarditis, autoimmune oophoritis, autoimmune orchitis, autoimmune pancreatitis, autoimmune retinopathy, Balo disease, Behcet's disease, benign mucosal pemphigoid, bullous pemphigoid, Castleman disease, Chagas disease, chronic inflammatory demyelinating polyneuropathy, chronic recurrent multifocal osteomyelitis, Churg-Strauss syndrome, cicatricial pemphigoid, coeliac disease, Cogan's syndrome, cold agglutinin disease, congenital heart block, Coxsackie myocarditis, CREST syndrome, dermatitis herpetiformis, Devic's disease, discoid lupus, Dressier's syndrome, endometriosis, eosinophilic esophagitis, eosinophilic fasciitis, erythema nodosum, essential mixed cryoglobulinemia, Evans syndrome, fibromyalgia, fibrosing alveolitis, giant cell arteritis, giant cell myocarditis, glomerulonephritis, Goodpasture's syndrome, granulomatosis with polyangiitis, Grave's disease, Guillain-Barre syndrome, haemolytic anaemia, Hashimoto's disease, Henoch-Schonlein purpura, herpes gestationis, hidradenitis suppurativa, hypogammaglobulinemia, idiopathic thrombocytopenic purpura, IgA nephropathy, IgG4-related sclerosing disease, immune thrombocytopenic purpura, inclusion body myositis, inflammatory bowel diseases, inflammatory myopathies, interstitial cystitis, juvenile arthritis, juvenile myositis, Kawasaki disease, Lambert-Eaton syndrome, leukocytoclastic vasculitis, lichen planus, lichen sclerosus, ligneous conjunctivitis, linear IgA disease, lupus, Lyme disease chronic, Meniere's disease, microscopic polyangiitis, mixed connective tissue disease, Mooren's ulcer, Mucha-Habermann disease, multifocal motor neuropathy, multiple sclerosis, myasthenia gravis, myositis, narcolepsy, neonatal lupus, neuromyelitis optica, neutropenia, ocular cicatricial pemphigoid, optic neuritis, palindromic rheumatism, paraneoplastic cerebellar degeneration, paroxysmal nocturnal hemoglobinuria, Parry Romberg syndrome, pars planitis, Parsonnage-Turner syndrome, pediatric autoimmune neuropsychiatry disorders associated with streptococcal infections (PANDAS), pemphigus, peripheral neuropathy, perivenous encephalomyelitis, pernicious anemia, POEMS syndrome, polyarteritis nodosa, polyglandular syndromes, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Sucac's syndrome sympathetic ophtalmia, systemic lupus erythematosus, Takayasu's arteritis, temporal arteritis, Tolosa-Hunt syndrome, transverse myelitis, type 1 diabetes, undifferentiated connective tissue disease, uveitis, vasculitis Vogt-Koyanagi-Harada disease, vitiligo and Wegener's granulomatosis.

### II. Disruption of a Target Gene(s)—METRNL, CXCR6 and/or CXCL16

As described herein, in certain embodiments, the expression of METRNL, CXCR6 and/or CXCL16 is disrupted. For example, the expression of METRNL, CXCR6 and/or CXCL16 can be disrupted in a T cell, more specifically, a CART cell. Gene suppression can be accomplished in a number of ways. For example, gene expression can be suppressed by knock out, altering a promoter of a gene, and/or by administering interfering RNAs. This can be done at an organism level or at a tissue, organ, and/or cellular level. If one or more genes are knocked down in a cell, tissue, and/or organ, the one or more genes can be suppressed by administrating RNA interfering reagents, e.g., siRNA, shRNA, or microRNA. For example, a nucleic acid which can express shRNA can be stably transfected into a cell to knockdown expression. Furthermore, a nucleic acid which can express shRNA can be inserted into the genome of a T cell, thus knocking down a gene within the T cell.

Disruption methods can also comprise overexpressing a dominant negative protein. This method can result in overall decreased function of a functional wild-type gene. Additionally, expressing a dominant negative gene can result in a phenotype that is similar to that of a knockout and/or knockdown.

In certain embodiments, a stop codon can be inserted or created (e.g., by nucleotide replacement), in the METRNL, CXCR6 and/or CXCL16 genes, which can result in a nonfunctional transcript or protein (sometimes referred to as knockout). For example, if a stop codon is created within the middle of one or more genes, the resulting transcription and/or protein can be truncated, and can be nonfunctional. However, in some cases, truncation can lead to an active (a partially or overly active) protein. If a protein is overly active, this can result in a dominant negative protein.

This dominant negative protein can be expressed in a nucleic acid within the control of any promoter. For example, a promoter can be a ubiquitous promoter. A promoter can also be an inducible promoter, tissue specific promoter, cell specific promoter, and/or developmental specific promoter. The nucleic acid that codes for a dominant negative protein can then be inserted into a cell. Any method can be used. For example, stable transfection can be used. Additionally, a nucleic acid that codes for a dominant negative protein can be inserted into a genome of a T cell.

One or more genes in a T cell, specifically, METRNL, CXCR6 and/or CXCL16, can be knocked out or disrupted using any method. For example, knocking out one or more genes can comprise deleting one or more genes including METRNL, CXCR6 and/or CXCL16 from a genome of a T cell. Knocking out can also comprise removing all or a part of a gene sequence from a T cell. It is also contemplated that knocking out can comprise replacing all or a part of a gene in a genome of a T cell with one or more nucleotides. Knocking out one or more genes can also comprise inserting a sequence in one or more genes thereby disrupting expression of the one or more genes. For example, inserting a sequence can generate a stop codon in the middle of one or more genes. Inserting a sequence can also shift the open reading frame of one or more genes.

The knockout of METRNL, CXCR6 and/or CXCL16 expression can be conditional. Conditional knockouts can be inducible, for example, by using tetracycline inducible promoters, development specific promoters. This can allow for eliminating or suppressing expression of a gene/protein at any time or at a specific time. For example, with the case of a tetracycline inducible promoter, tetracycline can be given to a T cell any time.

It is also contemplated that any combinations of knockout technology can be combined. For example, tissue specific knockout or cell specific knockout can be combined with inducible technology, creating a tissue specific or cell specific, inducible knockout. Furthermore, other systems such developmental specific promoter, can be used in combination with tissues specific promoters, and/or inducible knockouts.

Knocking out technology can also comprise gene editing. For example, gene editing can be performed using a nuclease, including CRISPR associated proteins (Cas proteins, e.g., Cas9), Zinc finger nuclease (ZFN), Transcription Activator-Like Effector Nuclease (TALEN), and meganucleases. Nucleases can be naturally existing nucleases, genetically modified, and/or recombinant. Gene editing can also be performed using a transposon-based system (e.g., PiggyBac, Sleeping beauty). For example, gene editing can be performed using a transposase.

Thus, as described herein, cells can be genetically altered ex vivo and used accordingly. These cells can be used for cell-based therapies. These cells can be used to treat disease in a recipient. For example, these cells can be used to treat cancer. In a particular embodiment, a method of treating a disease (e.g., cancer) in a recipient comprises transplanting to the recipient one or more cells comprising engineered cells. In more specific embodiments, about 5×10¹⁰ cells are administered to a subject. In some embodiments, about 5×10¹⁰ cells represents the median amount of cells administered to a subject. In some embodiments, about 5×10¹⁰ cells are necessary to effect a therapeutic response in a subject. In some embodiments, at least about at least about 1×10⁷ cells, at least about 2×10⁷ cells, at least about 3×10⁷ cells, at least about 4×10⁷ cells, at least about 5×10⁷ cells, at least about 6×10⁷ cells, at least about 6×10⁷ cells, at least about 8×10⁷ cells, at least about 9×10⁷ cells, at least about 1×10⁸ cells, at least about 2×10⁸ cells, at least about 3×10⁸ cells, at least about 4×10⁸ cells, at least about 5×10⁸ cells, at least about 6×10⁸ cells, at least about 6×10⁸ cells, at least about 8×10⁸ cells, at least about 9×10⁸ cells, at least about 1×10⁹ cells, at least about 2×10⁹ cells, at least about 3×10⁹ cells, at least about 4×10⁹ cells, at least about 5×10⁹ cells, at least about 6×10⁹ cells, at least about 6×10⁹ cells, at least about 8×10⁹ cells, at least about 9×10⁹ cells, at least about 1×10¹⁰ cells, at least about 2×10¹⁰ cells, at least about 3×10¹⁰ cells, at least about 4×10¹⁰ cells, at least about 5×10¹⁰ cells, at least about 6×10¹⁰ cells, at least about 6×10¹⁰ cells, at least about 8×10¹⁰ cells, at least about 9×10¹⁰ cells, at least about 1×10¹¹ cells, at least about 2×10¹¹ cells, at least about 3×10¹¹ cells, at least about 4×10¹¹ cells, at least about 5×10¹¹ cells, at least about 6×10¹¹ cells, at least about 6×10¹¹ cells, at least about 8×10¹¹ cells, at least about 9×10¹¹ cells, or at least about 1×10¹² cells. For example, about 5×10¹⁰ cells may be administered to a subject. In another example, starting with 3×10⁶ cells, the cells may be expanded to about 5×10¹⁰ cells and administered to a subject. In some cases, cells are expanded to sufficient numbers for therapy. For example, 5×10⁷ cells can undergo rapid expansion to generate sufficient numbers for therapeutic use. In some cases, sufficient numbers for therapeutic use can be 5×10¹⁰.

Any number of cells can be infused for therapeutic use. For example, a patient may be infused with a number of cells between 1×10⁶ to 5×10¹² inclusive. A patient may be infused with as many cells that can be generated for them. In some cases, cells that are infused into a patient are not all engineered. For example, at least 90% of cells that are infused into a patient can be engineered. In other instances, at least 85%, at least 80%, at least 75%, at least 70, at least 65%, at least 60%, at least 55%, at least 50%, at least 45%, or at least 40% of cells that are infused into a patient can be engineered.

The method disclosed herein can be used for treating or preventing disease including, but not limited to, cancer, autoimmune diseases and generally any disease or condition mediated, at least in part, by METRNL, CXCR6 and/or CXCL16.

### III. RNA Interference Compositions for Targeting METRNL, CXCR6 and/or CXCL16 mRNA

In one aspect of the present invention, the expression of METRNL, CXCR6 and/or CXCL16 may be inhibited by the use of RNA interference techniques (RNAi). RNAi is a remarkably efficient process whereby double-stranded RNA (dsRNA) induces the sequence-specific degradation of homologous mRNA in animals and plant cells. See Hutvagner and Zamore, 12 CURR. OPIN. GENET. DEV. 225-32 (2002); Hammond et al., 2 NATURE REV. GEN. 110-19 (2001); Sharp, 15 GENES DEV. 485-90 (2001). RNAi can be triggered, for example, by nucleotide (nt) duplexes of small interfering RNA (siRNA) (Chiu et al., 10 MOL. CELL. 549-61 (2002); Elbashir et al., 411 Nature 494-98 (2001)), micro-RNAs (miRNA), functional small-hairpin RNA (shRNA), or other dsRNAs which are expressed in-vivo using DNA templates with RNA polymerase III promoters. See, e.g., Zeng et al., 9 MOL. CELL. 1327-33 (2002); Paddison et al., 16 GENES DEV. 948-58 (2002); Lee et al., 20 NATURE BIOTECHNOL. 500-05 (2002); Paul et al., 20 NATURE BIOTECHNOL. 505-08 (2002); Tuschl, 20 NATURE BIOTECHNOL. 440-48 (2002); Yu et al., 99(9) PROC. NATL. ACAD. SCI. USA, 6047-52 (2002); McManus et al., 8 RNA 842-50 (2002); Sui et al., 99(6) PROC. NATL. ACAD. SCI. USA 5515-(2002).

As used herein, a METRNL, CXCR6 and/or CXCL16 inhibitory nucleic acid sequence can be a siRNA sequence or a miRNA sequence. An approximately 21-25 nucleotide siRNA or miRNA sequence can, for example, be produced from an expression vector by transcription of a short-hairpin RNA (shRNA) sequence, a 60-80 nucleotide precursor sequence, which is processed by the cellular RNAi machinery to produce either an siRNA or miRNA sequence. Alternatively, an approximately 21-25 nucleotide siRNA or miRNA sequence can, for example, be synthesized chemically. Chemical synthesis of siRNA or miRNA sequences is commercially available from such corporations as Dharmacon, Inc. (Lafayette, Colo.), Qiagen (Valencia, Calif.), and Ambion, Inc. (Austin, Tex.). An siRNA sequence preferably binds a unique sequence within the METRNL, CXCR6 or CXCL16 mRNA with exact complementarity and results in the degradation of the mRNA molecule. An siRNA sequence can bind anywhere within the mRNA molecule. An miRNA sequence preferably binds a unique sequence within the mRNA with exact or less than exact complementarity and results in the translational repression of the mRNA molecule. An miRNA sequence can bind anywhere within the mRNA molecule, but preferably binds within the 3′UTR of the mRNA molecule. Methods of delivering siRNA or miRNA molecules are known in the art. See, e.g., Oh and Park, Adv. Drug Deliv. Rev. 61(10):850-62 (2009); Gondi and Rao, J. Cell. Physiol. 220(2):285-91 (2009); and Whitehead et al., Nat. Rev. Drug Discov. 8(2)129-38 (2009).

As used herein, a METRNL, CXCR6 and/or CXCL16 inhibitory nucleic acid sequence can be an antisense nucleic acid sequence. Antisense nucleic acid sequences can, for example, be transcribed from an expression vector to produce an RNA which is complementary to at least a unique portion of the mRNA and/or the endogenous gene which encodes target protein. Hybridization of an antisense nucleic acid molecule under specific cellular conditions results in inhibition of the target protein expression by inhibiting transcription and/or translation.

A. Small Interfering RNA

In particular embodiments, the present invention features “small interfering RNA molecules” (“siRNA molecules” or “siRNA”), methods of making siRNA molecules and methods for using siRNA molecules (e.g., research and/or therapeutic methods). The siRNAs of this invention encompass any siRNAs that can modulate the selective degradation of METRNL, CXCR6 and/or CXCL16 mRNA.

In a specific embodiment, the siRNA of the present invention may comprise double-stranded small interfering RNA molecules (ds-siRNA). A ds-siRNA molecule of the present invention may be a duplex made up of a sense strand and a complementary antisense strand, the antisense strand being sufficiently complementary to a target mRNA to mediate RNAi. The siRNA molecule may comprise about 10 to about 50 or more nucleotides. More specifically, the siRNA molecule may comprise about 16 to about 30, e.g., 16, 17, 18, 19, 20, 21, 22, 23, 24, 25, 26, 27, 28, 29, or 30 nucleotides in each strand. The strands may be aligned such that there are at least 1, 2, or 3 bases at the end of the strands which do not align (e.g., for which no complementary bases occur in the opposing strand) such that an overhang of 1, 2 or 3 residues occurs at one or both ends of the duplex when strands are annealed.

In an alternative embodiment, the siRNA of the present invention may comprise single-stranded small interfering RNA molecules (ss-siRNA). Similar to the ds-siRNA molecules, the ss-siRNA molecule may comprise about 10 to about 50 or more nucleotides. More specifically, the ss-siRNA molecule may comprise about 15 to about 45 or more nucleotides. Alternatively, the ss-siRNA molecule may comprise about 19 to about 40 nucleotides. The ss-siRNA molecules of the present invention comprise a sequence that is “sufficiently complementary” to a target mRNA sequence to direct target-specific RNA interference (RNAi), as defined herein, e.g., the ss-siRNA has a sequence sufficient to trigger the destruction of the target mRNA by the RNAi machinery or process. In one embodiment, the ss-siRNA molecule can be designed such that every residue is complementary to a residue in the target molecule. Alternatively, substitutions can be made within the molecule to increase stability and/or enhance processing activity of the molecule. Substitutions can be made within the strand or can be made to residues at the ends of the strand. In a specific embodiment, the 5′-terminus may be phosphorylated (e.g., comprises a phosphate, diphosphate, or triphosphate group). In another embodiment, the 3′ end of an siRNA may be a hydroxyl group in order to facilitate RNAi, as there is no requirement for a 3′ hydroxyl group when the active agent is a ss-siRNA molecule. In other instances, the 3′ end (e.g., C3 of the 3′ sugar) of ss-siRNA molecule may lack a hydroxyl group (e.g., ss-siRNA molecules lacking a 3′ hydroxyl or C3 hydroxyl on the 3′ sugar (e.g., ribose or deoxyribose).

In another aspect, the siRNA molecules of the present invention may be modified to improve stability under in vitro and/or in vivo conditions, including, for example, in serum and in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference. For example, the absence of a 2′ hydroxyl may significantly enhance the nuclease resistance of the siRNAs in tissue culture medium.

Furthermore, the siRNAs of the present invention may include modifications to the sugar-phosphate backbone or nucleosides. These modifications can be tailored to promote selective genetic inhibition, while avoiding a general panic response reported to be generated by siRNA in some cells. In addition, modifications can be introduced in the bases to protect siRNAs from the action of one or more endogenous enzymes.

In an embodiment of the present invention, the siRNA molecule may contain at least one modified nucleotide analogue. The nucleotide analogues may be located at positions where the target-specific activity, e.g., the RNAi mediating activity is not substantially effected, e.g., in a region at the 5′-end and/or the 3′-end of the RNA molecule. Particularly, the ends may be stabilized by incorporating modified nucleotide analogues. Examples of nucleotide analogues include sugar- and/or backbone-modified ribonucleotides (e.g., include modifications to the phosphate-sugar backbone). For example, the phosphodiester linkages of natural RNA may be modified to include at least one of a nitrogen or sulfur heteroatom. In backbone-modified ribonucleotides, the phosphoester group connecting to adjacent ribonucleotides may be replaced by a modified group, e.g., a phosphothioate group. In sugar-modified ribonucleotides, the 2′ OH-group may be replaced by a group selected from H, OR, R, halo, SH, SR, NH₂, NHR, NR₂ or ON, wherein R is C₁-C₆ alkyl, alkenyl or alkynyl and halo is F, Cl, Br or I.

Nucleobase-modified ribonucleotides may also be utilized, e.g., ribonucleotides containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; 0- and N-alkylated nucleotides, e.g., N6-methyl adenosine are suitable. It should be noted that the above modifications may be combined.

Derivatives of siRNAs may also be utilized herein. For example, cross-linking can be employed to alter the pharmacokinetics of the composition, e.g., to increase half-life in the body. Thus, the present invention includes siRNA derivatives that include siRNA having two complementary strands of nucleic acid, such that the two strands are crosslinked. The present invention also includes siRNA derivatives having a non-nucleic acid moiety conjugated to its 3′ terminus (e.g., a peptide), organic compositions (e.g., a dye), or the like. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The siRNAs of the present invention can be enzymatically produced or totally or partially synthesized. Moreover, the siRNAs can be synthesized in vivo or in vitro. For siRNAs that are biologically synthesized, an endogenous or a cloned exogenous RNA polymerase may be used for transcription in vivo, and a cloned RNA polymerase can be used in vitro. siRNAs that are chemically or enzymatically synthesized are preferably purified prior to the introduction into the cell.

Although one hundred percent (100%) sequence identity between the siRNA and the target region is preferred in particular embodiments, it is not required to practice the invention. siRNA molecules that contain some degree of modification in the sequence can also be adequately used for the purpose of this invention. Such modifications may include, but are not limited to, mutations, deletions or insertions, whether spontaneously occurring or intentionally introduced.

Moreover, not all positions of a siRNA contribute equally to target recognition. In certain embodiments, for example, mismatches in the center of the siRNA may be critical and could essentially abolish target RNA cleavage. In other embodiments, the 3′ nucleotides of the siRNA do not contribute significantly to specificity of the target recognition. In particular, residues 3′ of the siRNA sequence which is complementary to the target RNA (e.g., the guide sequence) may not be critical for target RNA cleavage.

Sequence identity may be determined by sequence comparison and alignment algorithms known to those of ordinary skill in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (e.g., % homology=#of identical positions/total #of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (e.g., a local alignment). A non-limiting example of a local alignment algorithm utilized for the comparison of sequences is the algorithm of Karlin and Altschul, 87 PROC. NATL. ACAD. SCI. USA 2264-68 (1990), and as modified as in Karlin and Altschul 90 PROC. NATL. ACAD. SCI. USA 5873-77 (1993). Such an algorithm is incorporated into the BLAST programs (version 2.0) of Altschul, et al., 215 J. MOL. BIOL. 403-10 (1990).

In another embodiment, the alignment may be optimized by introducing appropriate gaps and determining percent identity over the length of the aligned sequences (e.g., a gapped alignment). To obtain gapped alignments for comparison purposes, Gapped BLAST can be utilized as described in Altschul et al., 25(17) NUCLEIC ACIDS RES. 3389-3402 (1997). In another embodiment, the alignment may be optimized by introducing appropriate gaps and determining percent identity over the entire length of the sequences aligned (e.g., a global alignment). A non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

In particular embodiments, greater than 90% sequence identity, e.g., 91%, 92%, 93%, 94%, 95%, 96%, 97%, 98%, 99% or even 100% sequence identity, between the siRNA and the portion of the target gene may be used. Alternatively, the siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). Additional hybridization conditions include, but are not limited to, hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length can be about 5-10° C. less than the melting temperature (Tm) of the hybrid, where Tm is determined according to the following equations. For hybrids less than 18 base pairs in length, Tm(° C.)=2(#of A+T bases)+4(#of G+C bases). For hybrids between 18 and 49 base pairs in length, Tm(° C.)=81.5+16.6(log 10[Na+])+0.41(% G+C)−(600/N), where N is the number of bases in the hybrid, and [Na⁺] is the concentration of sodium ions in the hybridization buffer ([Na⁺] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 50 or more bases.

B. Other Compositions for Targeting METRNL, CXCR6 and/or CXCL16 DNA or mRNA

Antisense molecules can act in various stages of transcription, splicing and translation to block the expression of a target gene. Without being limited by theory, antisense molecules can inhibit the expression of a target gene by inhibiting transcription initiation by forming a triple strand, inhibiting transcription initiation by forming a hybrid at an RNA polymerase binding site, impeding transcription by hybridizing with an RNA molecule being synthesized, repressing splicing by hybridizing at the junction of an exon and an intron or at the spliceosome formation site, blocking the translocation of an mRNA from nucleus to cytoplasm by hybridization, repressing translation by hybridizing at the translation initiation factor binding site or ribosome biding site, inhibiting peptide chain elongation by hybridizing with the coding region or polysome binding site of an mRNA, or repressing gene expression by hybridizing at the sites of interaction between nucleic acids and proteins. An example of an antisense oligonucleotide of the present invention is a cDNA that, when introduced into a cell, transcribes into an RNA molecule having a sequence complementary to at least part of the METRNL, CXCR6 and/or CXCL16 mRNA.

Furthermore, antisense oligonucleotides of the present invention include oligonucleotides having modified sugar-phosphodiester backbones or other sugar linkages, which can provide stability against endonuclease attacks. The present invention also encompasses antisense oligonucleotides that are covalently attached to an organic or other moiety that increase their affinity for a target nucleic acid sequence. For example, intercalating agents, alkylating agents, and metal complexes can be also attached to the antisense oligonucleotides of the present invention to modify their binding specificities.

The present invention also provides ribozymes as a tool to inhibit METRNL, CXCR6 and/or CXCL16 expression. Ribozymes are enzymatic RNA molecules capable of catalyzing the specific cleavage of RNA. The characteristics of ribozymes are well-known in the art. See, e.g., Rossi, 4 CURRENT BIOLOGY 469-71 (1994). Without being limited by theory, the mechanism of ribozyme action involves sequence specific hybridization of the ribozyme molecule to complementary target RNA, followed by an endonucleolytic cleavage. In particular embodiments, the ribozyme molecules include one or more sequences complementary to the target gene mRNA, and include the well-known catalytic sequence responsible for mRNA cleavage. See U.S. Pat. No. 5,093,246. Using the known sequence of the target mRNA, a restriction enzyme-like ribozyme can be prepared using standard techniques.

The expression of the METRNL, CXCR6 and/or CXCL16 genes can also be inhibited by using triple helix formation. Nucleic acid molecules to be used in triple helix formation for the inhibition of transcription can be single stranded and composed of deoxynucleotides. The base composition of these oligonucleotides must be designed to promote triple helix formation via Hoogsteen base paring rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarity to a purine-rich region of a single strand of the duplex in a parallel orientation to that strand. In addition, nucleic acid molecules that are purine-rich, e.g., containing a stretch of G residues, may be chosen. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′,3′-5′ manner, such that they base pair first with one strand of a duplex and then the other, eliminating the necessity for a sizeable stretch of either purines or pyrimidines to be present on one strand of a duplex.

The expression of METRNL, CXCR6 and/or CXCL16 may be also inhibited by what is referred to as “co-repression.” Co-repression refers to the phenomenon in which, when a gene having an identical or similar to the target sequence is introduced to a cell, expression of both introduced and endogenous genes becomes repressed. This phenomenon, although first observed in plant system, has been observed in certain animal systems as well. The sequence of the gene to be introduced does not have to be identical to the target sequence, but sufficient homology allows the co-repression to occur. The determination of the extent of homology depends on individual cases, and is within the ordinary skill in the art.

It would be readily apparent to one of ordinary skill in the art that other methods of gene expression inhibition that selectively target a METRNL, CXCR6 and/or CXCL16 DNA or mRNA can also be used in connection with this invention without departing from the spirit of the invention. In a specific embodiment, using techniques known to those of ordinary skill in the art, the present invention contemplates affecting the promoter region of METRNL, CXCR6 and/or CXCL16 to effectively switch off transcription.

C. Design and Production of the RNAi Compositions

One or more of the following guidelines may be used in designing the sequence of siRNA and other nucleic acids designed to bind to a target mRNA, e.g., shRNA, stRNA, antisense oligonucleotides, ribozymes, and the like, that are advantageously used in accordance with the present invention.

Beginning with the AUG start codon of the METRNL, CXCR6 and/or CXCL16 genes, each AA dinucleotide sequence and the 3′ adjacent 16 or more nucleotides are potential siRNA targets. In a specific embodiment, the siRNA is specific for a target region that differs by at least one base pair between the wild type and mutant allele or between splice variants. In dsRNAi, the first strand is complementary to this sequence, and the other strand identical or substantially identical to the first strand. siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in another embodiment, the nucleic acid molecules may have a 3′ overhang of 2 nucleotides, such as TT. The overhanging nucleotides may be either RNA or DNA. In one embodiment, it may be desirable to choose a target region wherein the mismatch is a purine:purine mismatch.

Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website (http://www.ncbi.nih.gov). Select one or more sequences that meet the criteria for evaluation.

Another method includes selecting in the sequence of the target mRNA, a region located from about 50 to about 100 nt 3′ from the start codon. In this region, search for the following sequences: AA(N19)TT or AA(N21), where N=any nucleotide. The GC content of the selected sequence should be from about 30% to about 70%, preferably about 50%. To maximize the specificity of the RNAi, it may be desirable to use the selected sequence in a search for related sequences in the genome of interest; sequences absent from other genes are preferred. The secondary structure of the target mRNA may be determined or predicted, and it may be preferable to select a region of the mRNA that has little or no secondary structure, but it should be noted that secondary structure seems to have little impact on RNAi. When possible, sequences that bind transcription and/or translation factors should be avoided, as they might competitively inhibit the binding of a siRNA, sbRNA or stRNA (as well as other antisense oligonucleotides) to the mRNA. Further general information about the design and use of siRNA may be found in “The siRNA User Guide,” available at The Max-Planck-Institut fur Biophysikalishe Chemie website (http://www.mpibpc.mpg.de).

Negative control siRNAs should have the same nucleotide composition as the selected siRNA, but without significant sequence complementarity to the appropriate genome. Such negative controls may be designed by randomly scrambling the nucleotide sequence of the selected siRNA; a homology search can be performed to ensure that the negative control lacks homology to any other gene in the appropriate genome.

D. Delivery of METRNL, CXCR6 and/or CXCL16 RNA Targeting Compositions

Delivery of the compositions of the present invention (e.g., siRNAs, antisense oligonucleotides, or other compositions described herein) into a patient can either be direct, e.g., the patient is directly exposed to the compositions of the present invention or compound-carrying vector, or indirect, e.g., cells are first transformed with the compositions of this invention in vitro, then transplanted into the patient for cell replacement therapy. These two approaches are known as in vivo and ex vivo therapy, respectively.

In the case of in vivo therapy, the compositions of the present invention are directly administered in vivo, where they are expressed to produce the encoded product. This can be accomplished by any of numerous methods known in the art, e.g., by constructing them as part of an appropriate nucleic acid expression vector and administering them so that they become intracellular, by infection using a defective or attenuated retroviral or other viral vector, by direct injection of naked DNA, by coating with lipids or cell-surface receptors or transfecting agents, encapsulation in liposomes, nanoparticles, microparticles, or microcapsules, by administering them in linkage to a peptide which is known to enter the cell or nucleus, or by administering them in linkage to a ligand subject to receptor-mediated endocytosis which can be used to target cell types specifically expressing the receptors. Further, the compositions of the present invention can be targeted in vivo for cell specific uptake and expression, by targeting a specific receptor. See, e.g., WO93/14188, WO 93/20221, WO 92/22635, WO92/20316, and WO 92/06180.

Ex vivo therapy involves transferring the compositions of the present invention to cells in tissue culture by methods well-known in the art such as electroporation, transfection, lipofection, microinjection, calcium phosphate mediated transfection, cell fusion, chromosome-mediated gene transfer, microcell-mediated gene transfer, spheroplast fusion, and infection with a viral vector containing the nucleic acid sequences. These techniques should provide for the stable transfer of the compositions of this invention to the cell, so that they are expressible by the cell and preferably heritable and expressible by its cell progeny. In particular embodiments, the method of transfer includes the transfer of a selectable marker to the cells. The cells are then placed under selection to isolate those cells that have taken up and are expressing the transferred compositions. The resulting recombinant cells can be delivered to a patient by various methods known in the art. Examples of the delivery methods include, but are not limited to, subcutaneous injection, skin graft, and intravenous injection.

### IV. Small Molecule Modulators of METRNL, CXCR6 and/or CXCL16

In one aspect, the methods of the present invention can be used to identify a METRNL, CXCR6 and/or CXCL16modulator. In particular embodiments, the METRNL, CXCR6 and/or CXCL16 modulator is a small molecule. The term “small molecule organic compounds” refers to organic compounds generally having a molecular weight less than about 5000, 4000, 3000, 2000, 1000, 800, 600, 500, 250 or 100 Daltons, preferably less than about 500 Daltons. A small molecule organic compound may be prepared by synthetic organic techniques, such as by combinatorial chemistry techniques, or it may be a naturally-occurring small molecule organic compound.

Nevertheless, compound libraries may be screened for METRNL, CXCR6 and/or CXCL16 modulators. A compound library is a mixture or collection of one or more putative modulators generated or obtained in any manner. Any type of molecule that is capable of interacting, binding or has affinity for METRNL, CXCR6 or CXCL16 may be present in the compound library. For example, compound libraries screened using this invention may contain naturally-occurring molecules, such as carbohydrates, monosaccharides, oligosaccharides, polysaccharides, amino acids, peptides, oligopeptides, polypeptides, proteins, receptors, nucleic acids, nucleosides, nucleotides, oligonucleotides, polynucleotides, including DNA and DNA fragments, RNA and RNA fragments and the like, lipids, retinoids, steroids, glycopeptides, glycoproteins, proteoglycans and the like; or analogs or derivatives of naturally-occurring molecules, such as peptidomimetics and the like; and non-naturally occurring molecules, such as “small molecule” organic compounds generated, for example, using combinatorial chemistry techniques; and mixtures thereof.

A library typically contains more than one putative modulator or member, i.e., a plurality of members or putative modulators. In certain embodiments, a compound library may comprise less than about 50,000, 25,000, 20,000, 15,000, 10000, 5000, 1000, 500 or 100 putative modulators, in particular from about 5 to about 100, 5 to about 200, 5 to about 300, 5 to about 400, 5 to about 500, 10 to about 100, 10 to about 200, 10 to about 300, 10 to about 400, 10 to about 500, 10 to about 1000, 20 to about 100, 20 to about 200, 20 to about 300, 20 to about 400, 20 to about 500, 20 to about 1000, 50 to about 100, 50 to about 200, 50 to about 300, 50 to about 400, 50 to about 500, 50 to about 1000, 100 to about 200, 100 to about 300, 100 to about 400, 100 to about 500, 100 to about 1000, 200 to about 300, 200 to about 400, 200 to about 500, 200 to about 1000, 300 to about 500, 300 to about 1000, 300 to 2000, 300 to 3000, 300 to 5000, 300 to 6000, 300 to 10,000, 500 to about 1000, 500 to about 2000, 500 to about 3000, 500 to about 5000, 500 to about 6000, or 500 to about 10,000 putative modulators. In particular embodiments, a compound library may comprise less than about 25,000, 20,000, 15,000, 10,000, 5,000, 1000, or 500 putative modulators.

A compound library may be prepared or obtained by any means including, but not limited to, combinatorial chemistry techniques, fermentation methods, plant and cellular extraction procedures and the like. A library may be obtained from synthetic or from natural sources such as for example, microbial, plant, marine, viral and animal materials. Methods for making libraries are well-known in the art. See, for example, E. R. Felder, Chimia 1994, 48, 512-541; Gallop et al., J. Med. Chem. 1994, 37, 1233-1251; R. A. Houghten, Trends Genet. 1993, 9, 235-239; Houghten et al., Nature 1991, 354, 84-86; Lam et al., Nature 1991, 354, 82-84; Carell et al., Chem. Biol. 1995, 3, 171-183; Madden et al., Perspectives in Drug Discovery and Design 2, 269-282; Cwirla et al., Biochemistry 1990, 87, 6378-6382; Brenner et al., Proc. Natl. Acad. Sci. USA 1992, 89, 5381-5383; Gordon et al., J. Med. Chem. 1994, 37, 1385-1401; Lebl et al., Biopolymers 1995, 37 177-198; and references cited therein. Compound libraries may also be obtained from commercial sources including, for example, from Maybridge, ChemNavigator.com, Timtec Corporation, ChemBridge Corporation, A-Syntese-Biotech ApS, Akos-SC, G & J Research Chemicals Ltd., Life Chemicals, Interchim S.A., and Spectrum Info. Ltd.

### V. Antibodies to METRNL, CXCR6 and/or CXCL16

The term antibody is used herein in a broad sense and includes both polyclonal and monoclonal antibodies. The term can also refer to a human antibody and/or a humanized antibody. Examples of techniques for human monoclonal antibody production include those described by Cole et al. (Monoclonal Antibodies and Cancer Therapy, Alan R. Liss, p. 77 (1985)) and by Boerner et al. (J. Immunol. 147(1):86-95 (1991)). Human antibodies (and fragments thereof) can also be produced using phage display libraries (Hoogenboom et al., J. Mol. Biol. 227:381 (1991); Marks et al., J. Mol. Biol. 222:581 (1991)). Human antibodies can also be obtained from transgenic animals. For example, transgenic mutant mice that are capable of producing a full repertoire of human antibodies, in response to immunization, have been described (see, e.g., Jakobovits et al., Proc. Natl. Acad. Sci. USA 90:2551-5 (1993); Jakobovits et al., Nature 362:255-8 (1993); Bruggermann et al., Year in Immunol. 7:33 (1993)).

Various procedures known in the art may be used for the production of antibodies to METRNL, CXCR6 and/or CXCL16, or any subunit thereof, or a fragment, derivative, homolog or analog of the protein. Antibodies of the present invention include, but are not limited to, synthetic antibodies, polyclonal antibodies, monoclonal antibodies, recombinantly produced antibodies, intrabodies, multispecific antibodies (including bi-specific antibodies), human antibodies, humanized antibodies, chimeric antibodies, synthetic antibodies, single-chain Fvs (scFv) (including bi-specific scFvs), single chain antibodies Fab fragments, F(ab′) fragments, disulfide-linked Fvs (sdFv), and anti-idiotypic (anti-Id) antibodies, and epitope-binding fragments of any of the above. In particular, antibodies of the present invention include immunoglobulin molecules and immunologically active portions of immunoglobulin molecules, e.g., molecules that contain an antigen binding site that immunospecifically binds to an antigen (e.g., one or more complementarity determining regions (CDRs) of an antibody).

Another embodiment for the preparation of antibodies according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993). The underlying rationale behind the use of peptide mimetics in rational design is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second generation molecules having many of the natural properties of the targeting antibodies disclosed herein, but with altered and even improved characteristics. More specifically, under this rational design approach, peptide mapping may be used to determine “active” antigen recognition residues, and along with molecular modeling and molecular dynamics trajectory analysis, peptide mimic of the antibodies containing antigen contact residues from multiple CDRs may be prepared.

In some embodiments, an antibody specifically binds an epitope of the METRNL, CXCR6 or CXCL16 protein. It is to be understood that the peptide regions may not necessarily precisely map one epitope, but may also contain a METRNL sequence that is not immunogenic. Methods of predicting other potential epitopes to which an immunoglobulin of the invention can bind are well-known to those of skill in the art and include, without limitation, Kyte-Doolittle Analysis (Kyte, J. and Dolittle, R. F., 157 J. MOL. BIOL. 105-32 (1982)); Hopp and Woods Analysis (Hopp, T. P. and Woods, K. R., 78 PROC. NATL. ACAD. SCI. USA 3824-28 (1981); Hopp, T. J. and Woods, K. R., 20 MOL. IMMUNOL. 483-89 (1983); Hopp, T. J., 88 J. IMMUNOL. METHODS 1-18 (1986)); Jameson-Wolf Analysis (Jameson, B. A. and Wolf, H., 4 COMPUT. APPL. BIOSCI. 181-86 (1988)); and Emini Analysis (Emini et al., 140 VIROLOGY 13-20 (1985)).

Amino acid sequence variants of the antibodies of the present invention may be prepared by introducing appropriate nucleotide changes into the polynucleotide that encodes the antibody or by peptide synthesis. Such modifications include, for example, deletions from, and/or insertions into and/or substitutions of, residues within the amino acid sequences of the antibody. Any combination of deletions, insertions, and substitutions may be made to arrive at the final construct.

Amino acid sequence insertions include amino-terminal and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody of a polypeptide that increases the serum half-life of the antibody.

Another type of antibody variant is an amino acid substitution variant. These variants have at least one amino acid residue in the antibody molecule replaced by a different residue. For example, the sites of greatest interest for substitutional mutagenesis of antibodies include the hypervariable regions, but framework region (FR) alterations are also contemplated.

A useful method for the identification of certain residues or regions of the METRNL, CXCR6 or CXCL16 antibodies that are preferred locations for substitution, i.e., mutagenesis, is alanine scanning mutagenesis. See Cunningham & Wells, 244 SCIENCE 1081-85 (1989). Briefly, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. The amino acid locations demonstrating functional sensitivity to the substitutions are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis may be conducted at the target codon or region and the expressed antibody variants screened for the desired activity.

Substantial modifications in the biological properties of the antibody can be accomplished by selecting substitutions that differ significantly in their effect on, maintaining (i) the structure of the polypeptide backbone in the area of the substitution, for example, as a sheet or helical conformation, (ii) the charge or hydrophobicity of the molecule at the target site, or (iii) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class. Conservative substitutions involve exchanging of amino acids within the same class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability, particularly where the antibody is an immunoglobulin fragment such as an FAT fragment.

Another type of substitutional variant involves substituting one or more hypervariable region residues of a parent antibody. Generally, the resulting variant(s), i.e., functional equivalents as defined above, selected for further development will have improved biological properties relative to the parent antibody from which they are generated. A convenient way for generating such substitutional variants is by affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed.

In order to identify candidate hypervariable region sites for modification, alanine-scanning mutagenesis may be performed to identify hypervariable region residues contributing significantly to antigen binding. Alternatively, or additionally, it may be beneficial to analyze a crystal structure of the antibody-antigen complex to identify contact points between the antibody and antigen. Such contact residues and neighboring residues are candidates for substitution according to the techniques elaborated herein. Once generated, the panel of variants is subjected to screening as described herein and antibodies with superior properties in one or more relevant assays may be selected for further development.

It may be desirable to modify the antibodies of the present invention, i.e., create functional equivalents, with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of an antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). Caron et al., 176 J. EXP MED. 1191-95 (1992); Shopes, 148 J. IMMUNOL. 2918-22 (1992). Homodimeric antibodies with enhanced anti-tumor activity may also be prepared using heterobifunctional cross-linkers as described in Wolff et al., 53 CANCER RESEARCH 2560-65 (1993). Alternatively, an antibody can be engineered which has dual Fc regions and may thereby have enhanced complement lysis and ADCC capabilities. Stevenson et al., 3 ANTI-CANCER DRUG DESIGN 219-30 (1989).

To increase the serum half-life of an antibody, one may incorporate a salvage receptor binding epitope into the antibody (especially an immunoglobulin fragment) as described in, for example, U.S. Pat. No. 5,739,277. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

Polynucleotide molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site directed) mutagenesis, PCR mutagenesis, and cassette mutagenesis of an earlier prepared variant or a non-variant version of the anti-METRNL, anti-CXCR6 and/or anti-CXCL16 antibodies of the present invention.

### VI. Chimeric Antigen Receptors (CARs)

In one aspect, the present invention provides CAR T therapy in which the expression of METRNL, CXCR6 and/or CXCL16 in the T cells is disrupted. In general, a CAR comprises at least one antigen binding domain, at least one transmembrane domain, and at least one intracellular domain. More specifically, a chimeric antigen receptor (CAR) is an artificially constructed hybrid protein or polypeptide containing the antigen binding domains of an antibody (e.g., single chain variable fragment (ScFv)) linked to T-cell signaling domains via the transmembrane domain. Characteristics of CARs include their ability to redirect T-cell specificity and reactivity toward a selected target in a non-MHC-restricted manner, and exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

In certain embodiments, the intracellular T cell signaling domains of the CARs can include, for example, a T cell receptor signaling domain, a T cell costimulatory signaling domain, or both. The T cell receptor signaling domain refers to a portion of the CAR comprising the intracellular domain of a T cell receptor. The costimulatory signaling domain refers to a portion of the CAR comprising the intracellular domain of a costimulatory molecule, which is a cell surface molecule other than an antigen receptor or their ligands that are required for an efficient response of lymphocytes to antigen.

A. Extracellular Domain

In one embodiment, a CAR comprises a target-specific binding element otherwise referred to as an antigen binding domain or moiety. The choice of domain depends upon the type and number of ligands that define the surface of a target cell. For example, the antigen binding domain may be chosen to recognize a ligand that acts as a cell surface marker on target cells associated with a particular disease state.

In one embodiment, the CAR can be engineered to target a tumor antigen of interest by way of engineering a desired antigen binding domain that specifically binds to an antigen on a tumor cell. Tumor antigens are proteins that are produced by tumor cells that elicit an immune response, particularly T-cell mediated immune responses. The selection of the antigen binding domain will depend on the particular type of cancer to be treated. Tumor antigens include, for example, a glioma-associated antigen, carcinoembryonic antigen (CEA), (3-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1 a, p53, prostein, PSMA, Her2/neu, survivin and telomerase, prostate-carcinoma tumor antigen-I (PCTA-1), MAGE, ELF2M, neutrophil elastase, ephrinB2, CD20, CD22, RORI, insulin growth factor (IGF)-I, IGF-11, IGF-I receptor and CD19. The tumor antigens disclosed herein are merely included by way of example. The list is not intended to be exclusive and further examples will be readily apparent to those of skill in the art.

The type of tumor antigen may also be a tumor-specific antigen (TSA) or a tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSAs or TAAs include the following: Differentiation antigens such as MART-I/MelanA (MART-I), gplOO (Pmel I 7), tyrosinase, TRP-I, TRP-2 and tumor-specific multi-lineage antigens such as MAGE-I, MAGE-3, BAGE, GAGE-I, GAGE-2, pI5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-I 80, MAGE-4, MAGE-5, MAGE-6, RAGE, NY-ESO, pI85erbB2, pI80erbB-3, c-met, nm-23HI, PSA, TAG-72, CA 19-9, CA 72-4, CAM 17.1, NuMa, K-ras, beta-Catenin, CDK4, Mum-I, p IS, p 16, 43-9F, 5T4, 79ITgp72, alpha-fetoprotein, beta-HCG, BCA225, BTAA, CA 125, CA I5-3\CA 27.29\BCAA, CA 195, CA 242, CA-50, CAM43, CD68\PI, CO-029, FGF-5, G250, Ga733\EpCAM, HTgp-175, M344, MA-50, MG7-Ag, MOVIE, NB/70K, NY-CO-I, RCASI, SDCCAGI 6, TA-90\Mac-2 binding protein\cyclophilin C-associated protein, TAAL6, TAG72, TLP, and TPS.

In one embodiment, the antigen binding domain portion of the CAR targets an antigen that includes but is not limited to CDI9, CD20, CD22, RORI, CD33, CD38, CDI23, CDI 38, BCMA, c-Met, PSMA, Glycolipid F77, EGFRvIII, GD-2, FGFR4, TSLPR, NY-ESO-I TCR, MAGE A3 TCR, and the like.

B. Transmembrane Domain

With respect to the transmembrane domain, a CAR comprises one or more transmembrane domains fused to the extracellular antigen binding domain of the CAR. The transmembrane domain may be derived either from a natural or from a synthetic source. Where the source is natural, the domain may be derived from any membrane-bound or transmembrane protein.

Transmembrane regions of particular use in the CARs described herein may be derived from (i.e., comprise at least the transmembrane region(s) of), but are not limited to, the alpha, beta or zeta chain of the T-cell receptor, CD2S, CD3 epsilon, CD45, CD4, CDS, CDS, CD9, CD16, CD22, mesothelin, CD33, CD37, CD64, CDSO, CDS3, CDS6, CD134, CD137, CD154, TNFRSF16, or TNFRSF19.

Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In one embodiment, the transmembrane domain that naturally is associated with one of the domains in the CAR is used in addition to the transmembrane domains described herein.

In some instances, the transmembrane domain can be selected or by amino acid substitution to avoid binding of such domains to the transmembrane domains of the same or different surface membrane proteins to minimize interactions with other members of the receptor complex.

C. Spacer (Hinge, H) Domain

In certain CAR embodiments, a spacer domain can be arranged between the extracellular domain and the transmembrane domain, or between the intracellular domain and the transmembrane domain. The spacer domain means any oligopeptide or polypeptide that serves to link the transmembrane domain with the extracellular domain and/or the transmembrane domain with the intracellular domain. The spacer domain comprises up to 300 amino acids, preferably 10 to 100 amino acids, and most preferably 25 to 50 amino acids.

In several embodiments, the linker can include a spacer element which, when present, increases the size of the linker such that the distance between the effector molecule or the detectable marker and the antibody or antigen binding fragment is increased. Exemplary spacers are known to the person of ordinary skill, and include those listed in U.S. Pat. Nos. 7,964,566; 7,495,295; 6,554,569; 6,323,315; 6,239,104; 6,034,065; 5,665,560; 5,663,149; 5,635,453; 5,599,902; 5,554,725; 5,530,097; 5,521,254; 5,410,024; 5,135,036; 5,076,973, 4,956,955; 4,975,744; 4,579,275; 4,516,444; and 4,45,414, as well as U.S. Pat. Pub. Nos. 20110212055 and 20110070245, each of which is incorporated by reference herein in its entirety.

In particular embodiments, the spacer domain comprises a sequence that promotes binding of a CAR with an antigen and enhances signaling into a cell. Examples of an amino acid that is expected to promote the binding include cysteine, a charged amino acid, and serine and threonine in a potential glycosylation site, and these amino acids can be used as an amino acid constituting the spacer domain.

In other embodiments, a spacer domain can comprise elements of Immunoglobulin (lg) constant domains including sequences that link immunoglobulin domains that comprise an immunoglobulin protein.

In further CAR embodiments, a signal peptide sequence can be linked to the N-terminus. The signal peptide sequence exists at the N-terminus of many secretory proteins and membrane proteins, and has a length of about 15 to about 30 amino acids. Because many of the protein molecules mentioned above as the intracellular domain have signal peptide sequences, the signal peptides can be used as a signal peptide for the CAR.

D. Intracellular Domain

The cytoplasmic domain or otherwise the intracellular signaling domain of the CAR is responsible for activation of at least one of the normal effector functions of the immune cell in which the CAR has been placed. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

E. Additional Description of CARs

Also expressly included within the scope of the invention are functional portions of CARs. The term “functional portion” when used in reference to a CAR refers to any part or fragment of one or more of a CAR, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to the parent CAR, the functional portion can comprise, for instance, about 10%, 25%, 30%, 50%, 68%, 80%, 90%, 95%, or more, of the parent CAR

The functional portion can comprise additional amino acids at the amino or carboxy terminus of the portion, or at both termini, which additional amino acids are not found in the amino acid sequence of the parent CAR Desirably, the additional amino acids do not interfere with the biological function of the functional portion, e.g., recognize target cells, detect cancer, treat or prevent cancer, etc. More desirably, the additional amino acids enhance the biological activity, as compared to the biological activity of the parent CAR.

Included in the scope of the disclosure are functional variants of the CARs disclosed herein. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR In reference to the parent CAR, the functional variant can, for instance, be at least about 30%, 50%, 75%, 80%, 90%, 98% or more identical in amino acid sequence to the parent CAR.

A functional variant can, for example, comprise the amino acid sequence of the parent CAR with at least one conservative amino acid substitution. Alternatively, or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Amino acid substitutions of the CARs are preferably conservative ammo acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, He, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gin, Ser, Thr, Tyr, etc.), an amino acid with a beta-branched side-chain substituted for another amino acid with a beta-branched side-chain (e.g., He, Thr, and Val), an amino acid with an aromatic side-chain substituted for another amino acid with an aromatic side chain (e.g., His, Phe, Trp, and Tyr), etc.

The CARs (including functional portions and functional variants) can be of any length, i.e., can comprise any number of amino acids, provided that the CARs (or functional portions or functional variants thereof) retain their biological activity, e.g., the ability to specifically bind to antigen, detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

The CARs (including functional portions and functional variants of the invention) can comprise synthetic amino acids in place of one or more naturally-occurring amino acids. Such synthetic amino acids are known in the art.

The CARs (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The CARs (including functional portions and functional variants thereof) can be obtained by methods known in the art. The CARs may be made by any suitable method of making polypeptides or proteins. Suitable methods of de nova synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; Epitope Mapping, ed. Westwood et al., Oxford University Press, Oxford, United Kingdom, 2001; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N Y 2001; and Ausubel et al., Current Protocols in Molecular Biology, Greene Publishing Associates and John Wiley & Sons, N Y, 1994. Further, some of the CARs (including functional portions and functional variants thereof) can be isolated and/or purified from a source, such as a plant, a bacterium, an insect, a mammal, e.g., a rat, a human, etc. Methods of isolation and purification are well-known in the art. Alternatively, the CARs described herein (including functional portions and functional variants thereof) can be commercially synthesized by companies. In this respect, the CARS can be synthetic, recombinant, isolated, and/or purified.

### VII. Pharmaceutical Compositions and Administration

Accordingly, a pharmaceutical composition of the present invention may comprise an effective amount of a METRNL, CXCR6 and/or CXCL16 modulator. As used herein, the term “effective,” means adequate to accomplish a desired, expected, or intended result. More particularly, an “effective amount” or a “therapeutically effective amount” is used interchangeably and refers to an amount of a METRNL, CXCR6 and/or CXCL16 modulator, perhaps in further combination with yet another therapeutic agent, necessary to provide the desired “treatment” (defined herein) or therapeutic effect, e.g., an amount that is effective to prevent, alleviate, treat or ameliorate symptoms of a disease or prolong the survival of the subject being treated. In particular embodiments, the pharmaceutical compositions of the present invention are administered in a therapeutically effective amount to treat patients suffering from cancer. As would be appreciated by one of ordinary skill in the art, the exact low dose amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

The pharmaceutical compositions of the present invention are in biologically compatible form suitable for administration in vivo for subjects. The pharmaceutical compositions can further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which a modulator is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain minor amounts of wetting or emulsifying agents, or pH buffering agents.

The pharmaceutical compositions of the present invention can take the form of solutions, suspensions, emulsions, tablets, pills, capsules, powders, sustained-release formulations and the like. The composition can be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount of a modulator together with a suitable amount of a pharmaceutically acceptable carrier so as to provide the form for proper administration to the patient. The formulation should suit the mode of administration.

The pharmaceutical compositions of the present invention may be administered by any particular route of administration including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. Most suitable routes are oral administration or injection. In certain embodiments, subcutaneous injection is preferred.

In general, the pharmaceutical compositions comprising a METRNL, CXCR6 and/or CXCL16 modulator may be used alone or in concert with other therapeutic agents at appropriate dosages defined by routine testing in order to obtain optimal efficacy while minimizing any potential toxicity. The dosage regimen utilizing a pharmaceutical composition of the present invention may be selected in accordance with a variety of factors including type, species, age, weight, sex, medical condition of the patient; the severity of the condition to be treated; the route of administration; the renal and hepatic function of the patient; and the particular pharmaceutical composition employed. A physician of ordinary skill can readily determine and prescribe the effective amount of the pharmaceutical composition (and potentially other agents including therapeutic agents) required to prevent, counter, or arrest the progress of the condition.

Optimal precision in achieving concentrations of the therapeutic regimen (e.g., pharmaceutical compositions comprising a modulator, optionally in combination with another therapeutic agent) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical compositions and various therapeutic agents may be independently optimized and combined to achieve a synergistic result wherein the pathology is reduced more than it would be if either was used alone.

In particular, toxicity and therapeutic efficacy of a pharmaceutical composition disclosed herein may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD₅₀/ED₅₀. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO 00/67776, which is entirely expressly incorporated herein by reference.

In particular embodiments, the compositions described herein, including engineered T cells, can be co-administered with one or more chemotherapeutic agents or chemotherapeutic compounds.

A “chemotherapeutic agent” or “chemotherapeutic compound” and their grammatical equivalents as used herein, can be a chemical compound useful in the treatment of cancer. The chemotherapeutic cancer agents that can be used in combination with a T cell include, but are not limited to, mitotic inhibitors (vinca alkaloids). These include vincristine, vinblastine, vindesine and Navelbine™ (vinorelbine, 5′-noranhydroblastine). In yet other cases, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that can be used in the methods and compositions disclosed herein are podophyllotoxin derivatives, such as etoposide, teniposide and mitopodozide. The present disclosure further encompasses other chemotherapeutic cancer agents known as alkylating agents, which alkylate the genetic material in tumor cells. These include, without limitation, cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that may be used in the methods and compositions disclosed herein include antibiotics. Examples include without limitation doxorubicin, bleomycin, dactinomycin, daunorubicin, mithramycin, mitomycin, mytomycin C, and daunomycin. There are numerous liposomal formulations commercially available for these compounds. The present disclosure further encompasses other chemotherapeutic cancer agents including without limitation anti-tumor antibodies, dacarbazine, azacytidine, amsacrine, melphalan, ifosfamide and mitoxantrone.

A composition, including an engineered T cell, can be administered in combination with other anti-tumor agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide.

Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 interleukin 12, retinoic acid, and tissue inhibitors of metalloproteinase-1 and -2 (TIMP-1 and -2). Small molecules, including topoisomerases such as razoxane, a topoisomerase II inhibitor with anti-angiogenic activity, can also be used.

Other anti-cancer agents that can be used in combination with the compositions described herein, including an engineered T cell, include, but are not limited to, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; avastin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idambicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclambicin; acylfulvene; adecypenol; adozelesin; aldesleukin; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-I; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor I-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; R11 retinamide; rogletimide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

For example, a pharmaceutical composition, including an engineered T cell, can be administered to a patient in conjunction with (e.g., before, simultaneously, or following) any number of relevant treatment modalities, including but not limited to treatment with agents such as antiviral therapy, cidofovir and interleukin-2, or Cytarabine (also known as ARA-C). In some cases, a pharmaceutical composition, including an engineered T cell, can be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. A pharmaceutical composition, including an engineered T cell, can also be administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some cases, a pharmaceutical composition, including an engineered T cell, can be administered following B-cell ablative therapy such as agents that react with CD20, e.g., Rituxan. For example, subjects can undergo standard treatment with high dose chemotherapy followed by peripheral blood stem cell transplantation. In certain cases, following the transplant, subjects can receive a pharmaceutical composition, including an infusion of the engineered cells, e.g., expanded engineered cells, of the present invention. Additionally, a pharmaceutical composition, including expanded engineered T cells, can be administered before or following surgery.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: MATRNL is an Immunosuppressive Cytokine in CD8 Tumor-Infiltrating Lymphocytes

Checkpoint inhibitors (CI) mitigate T cell exhaustion to achieve durable remissions in some patients with advanced solid tumors [1]. However, many patients with CI-sensitive pathologies, such as melanoma [2], non-small cell lung cancer (NSCLC) [3], renal cell carcinoma (RCC) [4], and urothelial carcinoma [5], fail to derive clinical benefit. Apart from hypermutated tumors [6,7], objective responses in CI-insensitive pathologies, such as glioblastoma (GBM), are infrequent and lack durability [8,9]. Immunotherapy resistance can be primary, adaptive, or acquired. Primary immunotherapy resistance mechanisms stem from a paucity of clonal mutations that generate mutation-associated neoantigens, poor HLA affinity of available neoantigens, impaired antigen presentation, and altered interferon responses [10]. Adaptive resistance counters an antitumor immune response via upregulation of immune checkpoints [11] as well as recruitment of suppressive immune cell populations [12]. Acquired resistance can encompass multiple changes in gene programs among different cellular components of the tumor microenvironment arising under immunologic pressure [13]. The relative roles of each of these mechanisms in tumor progression are not yet fully understood and may vary based on pathology. Therefore, identifying universal immunosuppressive pathways in cancer is of interest for informing more effective immunotherapeutic strategies.

The present inventors used a comparative transcriptomics approach to identify genes differentially expressed among tumor-infiltrating lymphocytes (TILs) and peripheral blood lymphocytes (PBLs) collected from patients harboring previously untreated tumors with different response rates to CIs: prostate cancer (n=12), GBM (n=8), RCC (n=8), and urothelial bladder carcinoma (n=8). To enrich for genes associated with exhaustion, the present inventors dichotomized high and low expression of programmed cell death protein 1 (PDCD 1 or PD-1), lymphocyte activating 3 (LAG-3), and hepatitis A virus cellular receptor 2 (HAVCR2 or TIM-3) within these datasets. An analysis of differentially expressed genes among checkpoint-high CD8 TILs vs. all other samples identified 13 genes of interest. Analysis of differentially expressed genes among checkpoint-high CD8 TILs vs. activated checkpoint-high CD8 PBLs identified 36 genes of interest. METRNL was present in both sets, indicating that it is co-expressed with immune checkpoints and independently associated with intratumoral location.

The METRNL gene is located on human chromosome 17q25.3 and mouse chromosome 11qE2 and encodes a small (˜30KD) protein homologous to Meteorin (METRN), which is involved in glial cell development [14]. Also known as Cometin, Subfatin or Metrnb, METRNL is a small, secreted protein with diverse functions in metabolism and immunity [14]. METRNL was identified as a novel adipokine based on a screen using diet-induced obese mice [15]. Rao and colleagues found that METRNL is secreted by skeletal muscle following exercise or cold exposure and improves glucose tolerance, induces expression of genes associated with beige fat thermogenesis, and stimulates alternative activation of adipose tissue macrophages via an eosinophil-dependent increase in IL-4 [16]. Subsequent work confirmed that METRNL increases expression of fatty acid oxidation-associated and anti-inflammatory genes via AMP-activated protein kinase (AMPK) or peroxisome proliferator activated receptor delta (PPARd) and is also highly expressed in barrier tissues by M2-polarized macrophages [18]. Metrnl KO mice exhibit alterations in IgM and IgG levels, are susceptible to LPS endotoxemia, and develop autoimmune lesions [19]. More recently, METRNL was shown to inhibit chronic inflammation and facilitate skeletal muscle repair [20]. A potential role in cancer was implicated by a TCGA screen that identified METRNL as being involved in one of 11 epigenetic interactions associated with survival in bladder cancer [21]. In vitro studies of pancreatic cancer cells have also suggested that METRNL may directly promote tumor cell proliferation [22]. However, a role for METRNL in immune-oncology has not been previously reported.

The present inventors confirmed that Metrnl is co-expressed with immune checkpoints in CD8 TILs isolated from a murine glioma model. Exogenous Metrnl suppressed CD8 T cell activation and effector function in vitro and in vivo. The present inventors found that Metrnl ablation improved anti-tumor immunity in murine models of glioma, prostate, and colorectal cancers in a CD8-dependent manner. Mechanistically, METRNL depolarized mitochondria, increased the concentration of reactive oxygen species (ROS), and induced apoptosis of CD8 T cells. Furthermore, a compensatory oxidative stress response to Metrnl exposure decreased glycolytic flux of activated CD8 T cells. Taken together, these results identify METRNL as a novel metabolic immune checkpoint and potential target for immunotherapy.

### Materials and Methods

Human Subjects/Lymphocyte sorting. All procedures were approved by the Johns Hopkins Institutional Review Board (IRB0049987, NA_00026693, NA_0082175, IRB00033839). Specimens were obtained from patients undergoing resection of previously untreated GBM, prostate cancer, bladder urothelial carcinoma, and RCC. T cells were isolated from tumor tissue (TIL) and peripheral blood (PBL) as previously described [64]. Enriched T-cells were stained with the following antibodies: CCR7, CD45RO, CD127, CD25, CD45RA, CD4, CD8, CD28 and CD27. Target populations were defined as the following: PBL CD4 naive (CD4+CD25LowCD127+/−CCR7+CD45RA+CD27+CD28+), PBL CD4 regulatory T-cell (Treg)(CD4+CD25HiCD127Low), PBL CD8 naive (CD8+CD45RA+CD45RO-CD27+CD28+), TIL CD4 Treg (CD4+CD25HiCD127Low), TIL CD8 antigen experienced (CD8+CD45RA-CD45R0+), PBL CD4 activated and PBL CD8 activated subgroups. For prostate tumors, PBL CD8 antigen experienced (CD8+CD45RA-CD45R0+) were also sorted and included in the analysis (FIG. S6A, B). Each population was sorted into 2 mL of complete RPMI media and separated into two fractions each; the first fraction of each was centrifuged at 400RCF for 10 minutes at RT then supernatant was discarded, and the cells were resuspended/lysed in 800 uL of Trizol Reagent LS (Thermo Fisher Scientific), the second fraction for each was then activated ex-vivo using anti-CD3/CD28 stimulatory beads for 72 hours. PBL CD4 regulatory T-cell, PBL CD8 antigen experienced, TIL CD4 Treg, TIL CD8 antigen experienced groups were each sorted directly into 800 uL of Trizol Reagent LS. Samples resuspended in Trizol Reagent LS were stored in a −80 C freezer until RNA extraction.

RNA extraction. Sample-containing trizol tubes were thawed at room temperature for approximately 10 minutes, after which 160 uL of chloroform was added to each tube. Tubes were mixed one at a time by inverting them for 15 seconds each. Using a pre-cooled microcentrifuge (4° C.), samples were centrifuged at maximum speed for twenty minutes. The chloroform layer was then removed and transferred to a new RNAse free 1.7 mL tube. 400 uL of 100% isopropanol and 2 uL of molecular grade glycogen (ThermoFisher Scientific) were added to each sample. Samples were individually mixed by inverting the tubes for 15 seconds and incubated for 10 minutes at room temperature. Samples were then centrifuged at 4° C. for 10 minutes at maximum speed. The supernatant was aspirated, with caution not to disturb the glycogen pellet. The tube and pellet were then washed with 70% EtOH, with effort to dislodge the pellet from the bottom of the tube but not to resuspend it in solution. Samples were centrifuged at maximum speed at 4° C. for 10 minutes. Supernatant was aspirated down to a few microliters; the remaining supernatant was aspirated with a small pipette tip. The pellet was then resuspended in 10 uL of RNase/DNase free water and tested with a bioanalyzer for RNA quality.

RNA sequencing. Libraries were prepared for all samples starting with 500 pg-100 ng of total RNA. cDNA was prepared as directed in the Nugen Ovation RNA-Seq System V2 Sample Preparation Guide. After fragmentation on the Covaris S2, adapter ligation and indexing were followed by PCR amplification to selectively enrich correctly ligated DNA fragments. Libraries were run on a High Sensitivity chip using the Agilent Bioanalyzer to assess size distribution and overall quality of the amplified library. Quantification of the libraries was performed by qPCR with the Kappa Library Quantification Kit or by the Agilent Bioanalyzer. Equimolar concentrations of each library were pooled together.

Sequencing was performed in multiple batches with two sequencers. For some samples, cluster generation and sequencing were performed on an Illumina HS2000 platform for a 100 bp×100 bp, paired end sequencing utilizing the TruSeq PE Cluster Kit v3 and TruSeq SBS Kit v3 (200 cycles). For other samples, cluster generation and sequencing were performed on an Illumina HS2500 platform for a 100 bp×100 bp, paired end sequencing utilizing the TruSeq Rapid PE Cluster Kit and TruSeq Rapid SBS Kit (200 cycles).

Samples were aligned and gene transcript expression levels were generated using rsem-1.2.29. The ‘rsem-calculate-expression’ module was used with the following options: —bowtiechunkmbs 200, —calc-ci, —output-genome-bam, —paired-end and —forward-prob 0.5. The data was aligned to “hg19” human reference genome. Both RefSeq gene annotations and Illumina iGenomes annotation were used as annotations. For each study, the present inventors selected the genes in common resulting from the two annotations.

RNA data analysis. The present inventors performed a meta-analysis of four different RNA-Seq studies of prostate cancer, GBM, RCC, and bladder cancer TILs and matched PBLs data. Each study was carried out at the sequencing core at Sidney Kimmel Comprehensive Cancer Center at Johns Hopkins. The alignments were performed using a standardized pipeline including bowtie for alignment and RSEM for quantification of the transcriptome. Given that the four studies were carried out at separate times and, considering that different reference genomes were used within and between studies, in the meta-analysis the present inventors subjected each study to the same bioinformatics analysis and compared the final level differential expression analysis results.

With some of the cohorts, two different references genomes (hg19) were used for alignment and therefore the genes in common were selected. Estimation of gene level expression counts were performed using RSEM, resulting in 26341, 34609, 22845, and 22885 gene level identifiers for the GBM, PRAD, RCC and BLCA cohorts respectively, with 21204 genes in common. The final sample counts (including both CD4 and CD8) were 96 for PRAD and 56 for GBM, RCC and BLCA. The present inventors worked with the RSEM estimated count data from hereon. A differential expression analysis between the antigen-experienced CD8 tumor group and the activated CD8 PBL group was carried out with the limma package. Zero expression genes were removed from the analysis and further, only genes where the count per million value was greater than 1 more than 1 sample were retained from each cohort. Following this, the voom transformation was used for normalization of the data prior to the t-test based differential expression analysis with the limma package on the R platform.

For dichotomizing the samples into positive and negative states for PD-1, TIM-3, and LAG-3, the present inventors used the expectation maximization algorithm. The expression values for each marker were supplied to the algorithm and fitter to obtain two or three normal distributions that would approximate the overall distribution of the gene. The cutpoint between the two distributions (if the algorithm selected three distributions, then two of these were selected visually) was computed and selected as the threshold for deciding whether each sample had a positive or negative status with respect to the immune checkpoint marker. The other differential expression analyses were carried out in the same manner as the previous, using the voom transformation and the linear model fitting provided by limma. Gene Set Enrichment Analysis (GSEA) was performed as previously described using the Functional Gene Set (FGS) collections from the Broad Institute Molecular Signature Database (MSigDB).

The differential expression analysis was carried out by fitting a linear model with only gene expression data as variables. A variance stabilizing transformation was applied to the gene expression data prior to this model fitting. Generally, the present inventors can state the model for each gene in the form of

y=Xb+e

with y being the observed outcome (immune marker status sub-group), X being the design matrix, and e being the error, with b being estimated.

Immunohistochemistry. Fresh frozen paraffin embedded (FFPE) GBM specimens were stained using rabbit anti-human METRNL antibody (Abcam, ab121775) according to the protocol provided by the manufacturer. Images were obtained using a Zeiss Axiocam (Zeiss, Germany).

Mice, cell lines, and cell lysates. Female C57BL/6J (Jackson Laboratory), B6.SJL-PtprcaPepcb/BoyJ (CD45.1) (Jackson Laboratory), and C57BL/6-Tg(TcraTcrb)1100Mjb/J (OT-1) (Jackson Laboratory) mice (6-8 weeks) were housed in pathogen-free conditions under animal protocols approved by the Johns Hopkins University Institutional Animal Care and Use Committee (IACUC). CD45.1 mice were used as donors for dendritic cells in co-culture experiments. OT-1 mice were used as donors for CD3+CD8+OT-1 T cells for in vitro suppression assays. Metrnl KO mice (C57BL/6JMetrnitm1d(KOMP)Wtsi/MbpMmucd) were obtained from the Mutant Mouse Resource and Research Center (MMRC) at the University of California Davis. GL261-Luc2 cells were maintained in cell culture with medium DMEM+10% FBS+1% Penicillin/Streptomycin upon which they were either used for intracranial implantation or to produce tumor cell lysate with 1×RIPA buffer. MC38 cells were maintained in cell culture with medium DMEM+10% FBS+1% Penicillin/Streptomycin. B6CaP cells were propagated in vivo and isolated after surgical resection of flank tumor, mechanical disruption into single cell suspension using 70 μm cell strainer and CD45-depletion using CD45 microbeads.

Tumor models and TIL harvest. To establish glioma, 1.3e5 GL261-Luc2 cells were injected with a mouse stereotaxic frame at coordinates 1 mm anterolateral from bregma at a depth of 2 mm as previously described [66]. Mice were imaged for tumor burden on day 7 post-implantation with IVISR using 1 mg/kg injections of D-luciferin with luminescent imaging of luciferase activity. Mice with tumor signal on day 7 were segregated from mice without tumor signal and followed until day 21 with repeat IVISR imaging to confirm continued presence of tumor. On day 24, mice brains were harvested, mechanically dissociated, and strained through a 70μ.m filter, and spun down in a 30%/70% PercollR (Sigma-Aldrich) gradient at 2200 rpm for 20 minutes without brakes. Tumor infiltrating lymphocytes and myeloid populations were extracted and resuspended in phosphate buffered saline (Quality Biological, Gaithersburg, MD) for flow cytometric staining.

For flank tumors, 100,000 MC38 cells were injected into the right flank of 6-8 weeks old female C57BL/6J and Metrnl KO mice, and 100,000 B6CaP cell were injected into the right flank of male mice. Tumor volume was measured using calipers every few days after tumors became palpable. On day 16, mice were euthanized, and tumors were isolated, weighed, mechanically dissected and strained through a 70 μm filter. After multiple washes, cells were resuspended in phosphate buffered saline for further analysis.

For intratumoral injection of exogenous METRNL mixed with hydrogel, 800 uL of PBS was added to 200 mg of PLCL-PEG-PLCL hydrogel and mixed until fully dissolved, using a tube rotator. Exogenous METRNL was added to dissolved hydrogel before intratumoral injections to achieve 50 μg/ml concentration of METRNL. The admixture was injected into the site of MC38 flank tumors on day 8 after implantation.

Flow cytometric staining and sorting. Lymphocytes were stained for CD45, CD3, CD4, CD8, PD-1, TIM-3, and LAG-3 after L/D for excluding dead cells. Live CD45+CD3+CD8+ cells were sorted into triple negative (PD-1−, TIM-3−, LAG-3−), single positive (PD-1+, TIM-3−, LAG-3−), and triple positive (PD-1+, TIM-3+, LAG-3+) populations. The gating strategy is shown in FIG. S5A. For Annexin V and propidium iodide staining, TILs were stained with anti-CD45, anti-CD3, anti-CD8 antibodies for 15 minutes. Cells were stained with Annexin V and PI, diluted in annexin binding buffer for 15 min as per manufacturer's protocol.

IVIS imaging. For imaging luminescent activity of luciferase protein in mice, GL261-luc2 bearing mice were administered intraperitoneal D-luciferin (10 mg/kg). After 5 minutes mice were anesthetized in an induction chamber with an Isoflurane-02 gas mixture at 2.5 L/min. After achieving adequate anesthesia as noted by noxious stimuli of the hindpaw, mice were moved to the imaging chamber and remained anesthetized by continuous administration of Isoflurane-02 via nose cone.

TIL co-culture. For METRNL and IFN-gamma based assays with triple negative, single positive, and triple negative cells, 1e3 cells from each population were co-cultured with 100 μg/ml GL261-Luc2 tumor cell lysate and 5e3 dendritic cells isolated from 45.1 mouse spleen using a pan-dendritic cell isolation kits in 96-well round bottomed plates in T cell media (RPMI 1640+10% FBS+1% NEAA+1% 2-Mercaptoethanol+1% Penicillin/Streptomycin). Co-cultured cells were incubated at 37° C. for 48 hours with GolgiStop (BD Biosciences, Franklin Lakes, NJ) administered 6 hours prior to cell harvest. Supernatant was collected for subsequent ELISA for Mouse Meteorin-like/METRNL DuoSet (R&D Systems, Minneapolis, MN) and IFN-gamma (Thermo Fisher, Waltham, MA).

In vitro activation assay. For in vitro activation assays involving mouse METRNL, CD3+CD8+ OT-1 T cells were harvested from the spleen of OT-1 mice using CD8a+ isolation kits and were co-cultured with dendritic cells isolated from 45.1 mice spleens using pan-dendritic cell isolation kits. Each well was given 2 μM of OVA SIINFEKL peptide along with varying concentrations of recombinant mouse METRNL at 0, 0.5 μg/ml, 2.5 μg/ml, and 5 μg/ml. Co-cultured cells were incubated at 37° C. for 48 hours with GolgiStop administered 6 hours prior to cell harvest. Supernatant was collected for subsequent ELISA for IFN-gamma. Cells were collected from each well, stained for CD8, CD45.2, PD-1, LAG-3, and TIM-3 and analyzed by flow cytometry.

To determine proliferation of cancer cells in response to Metrnl, MC38 cells were plated with 0 μg/ml, 0.5 μg/ml, 2.5 μg/ml, and 5 μg/ml of METRNL in 96 well plates. 10 μL of AlamarBlue viability dye was added to the cells (as per manufacturer's instructions). Absorbance was measured with a microplate absorbance reader at 1, 2, 3 and 4 hours on days 1-3.

Mitochondrial staining and 2-NBDG labeling. For in vitro assessment of mitochondrial mass and membrane potential, 250,000 T cells were incubated in a 96-well plate with PMA/Ionomycin and increasing dose of exogenous METRNL for 6 hours. After the incubation period, the plate was centrifuged, and the supernatant discarded. Pelleted cells were resuspended with the following dyes, incubated at 37 C while protected from light-MitoSOX Red Mitochondrial Superoxide Indicator, 5 μM in PBS for 15 minutes; MitoTracker Red CMXRos, 1 μM in culture media for 15 minutes and Mitotracker Green, 50 nM in PBS for 15 minutes. After staining was complete, cells were re-pelleted and washed 2× with PBS. Cells were analyzed by flow cytometry as previously described [67]. Glucose uptake was measured by exposing cells that were plated for 3 days with METRNL to 50 μM 2-NBDG in glucose-free RPMI at 37 C for 15 minutes.

Targeted Metabolite analysis with LC-MS/MS. Targeted metabolite analysis was performed with liquid-chromatography tandem mass spectrometry (LC-MS/MS). Metabolites from cells (treated with METRNL for 4 days) were extracted with 80% (v/v) methanol solution equilibrated at −80° C., and the metabolite-containing supernatants were dried under nitrogen gas. Dried samples were re-suspended in 50% (v/v) acetonitrile solution and 4 ml of each sample were injected and analyzed on a 5500 QTRAP mass spectrometer (AB Sciex) coupled to a Prominence ultra-fast liquid chromatography (UFLC) system (Shimadzu). The instrument was operated in selected reaction monitoring (SRM) with positive and negative ion-switching mode as described. This targeted metabolomics method allows for analysis of over two hundred metabolites from a single 25-min LC-MS acquisition with a 3-ms dwell time and these analyzed metabolites cover all major metabolic pathways. The optimized MS parameters were: ESI voltage was +5,000V in positive ion mode and −4,500V in negative ion mode; dwell time was 3 ms per SRM transition and the total cycle time was 1.57 seconds. Hydrophilic interaction chromatography (HILIC) separations were performed on a Shimadzu UFLC system using an amide column (Waters XBridge BEH Amide, 2.1×150 mm, 2.5μ,m). The LC parameters were as follows: column temperature, 40° C.; flow rate, 0.30 ml/min. Solvent A, Water with 0.1% formic acid; Solvent B, Acetonitrile with 0.1% formic acid; A nonlinear gradient from 99% B to 45% B in 25 minutes with 5 min of post-run time. Peak integration for each targeted metabolite in SRM transition was processed with MultiQuant software (v2.1, AB Sciex). The preprocessed data with integrated peak areas were exported from MultiQuant and re-imported into Metaboanalyst software for further data analysis (e.g., statistical analysis, fold change, principle components analysis, etc.).

Imaging. Naive CD8 T cells were activated with anti-CD3/CD28 beads with or without 2.5 μg/ml of exogenous Metrnl for duration indicated in the respective figures. The images were captured using the Zeiss Observer Z1 microscope (Oberkochen, Germany). Images were collected using equal exposure times and processed in a similar fashion using the Zen 2.6 Blue program. At least three images were collected per treatment group. Each image was counted and analyzed for statistical significance. The experiments were conducted multiple times and the shown images are representative samples.

Depletion studies. WT and Metrnl KO mice received intraperitoneal (IP) injections of either isotype control, anti-CD4 or anti-CD8 depletion antibodies at 200 μg/dose, on days −1, 2, 5, 8 and 11 from MC38 flank tumor implantation. Depletion of CD4 and CD8 T cell populations was confirmed on day 7 by flow cytometry analysis of 2 mice harvested from each group. Depletion was >99% for both populations.

Statistical analysis. Statistical analyses were performed as mentioned in the FIG. legends for each experiment. As appropriate for the experiment, statistical significance was determined using either unpaired Student's t test when comparing two groups, one-way ANOVA to compare multiple groups and two-way ANOVA for repeated measurements. Mantel-Cox log-rank test was used to compare survival. Significance was defined as *p≤0.05; **p≤0.01; ***p≤0.001; ****p≤0.0001. All statistical analysis was calculated using Prism software (GraphPad).

### Results

Differential expression of selected genes validates the dataset. TILs and PBLs were isolated from patients with previously untreated GBM, prostate cancer, bladder urothelial carcinoma, and RCC, sorted based on activation status and antigen experience, and bulk RNA sequencing was performed. To validate the dataset, a subset of 14 genes previously characterized as markers of lymphocyte activation or exhaustion were selected [23,24] and the ranges of expression for the selected genes in GBM (A), PRAD (B), RCC (C), and BLCA (D) were determined (FIG. S1). These data confirmed that expression patterns of these genes behaved as expected. For example, interferon gamma IFNG and granzyme B GZMB (granzyme B) were highly expressed in activated CD8 PBL, with lower expression in antigen-experienced TIL. Forkhead box P3 (FOXP3) was highly expressed in Tregs in tissue and blood, with low expression in other populations. PD-1 was highly expressed on activated T cells with the highest expression levels on CD8 TILs. Cytotoxic T-lymphocyte associated protein 4 (CTLA-4) showed the highest expression on Treg TILs, with relatively high levels of expression on activated PBL and antigen-experienced CD8 TILs. LAG-3 and TIM-3 were highly expressed on activated CD4 and CD8 PBL and antigen-experienced TILs. These expression patterns were consistent across tumor types and confirm that TILs express high levels of genes associated with exhaustion and lower levels of genes associated with activation and cytotoxicity compared with activated PBL.

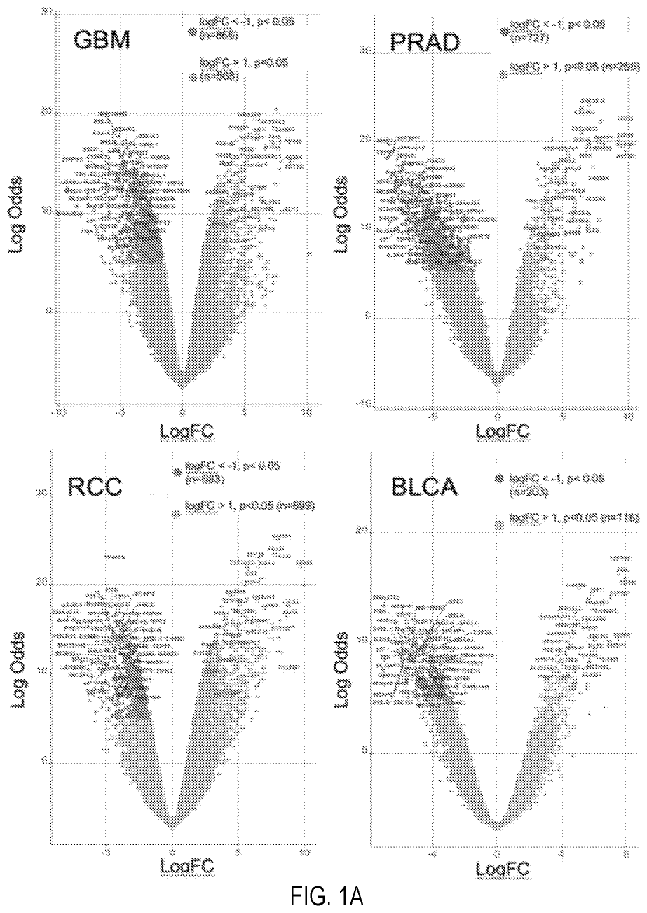

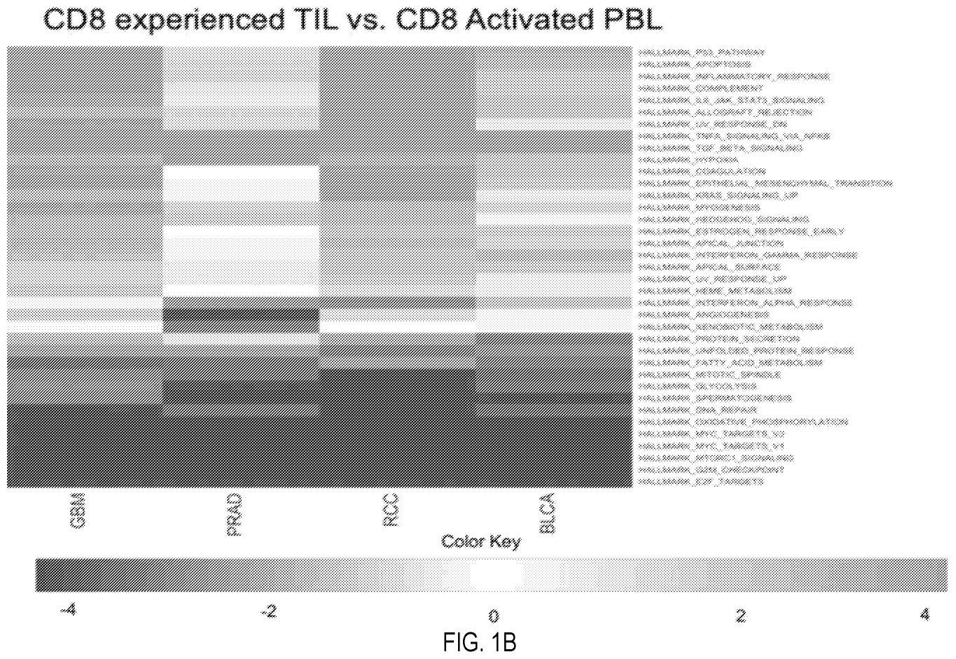

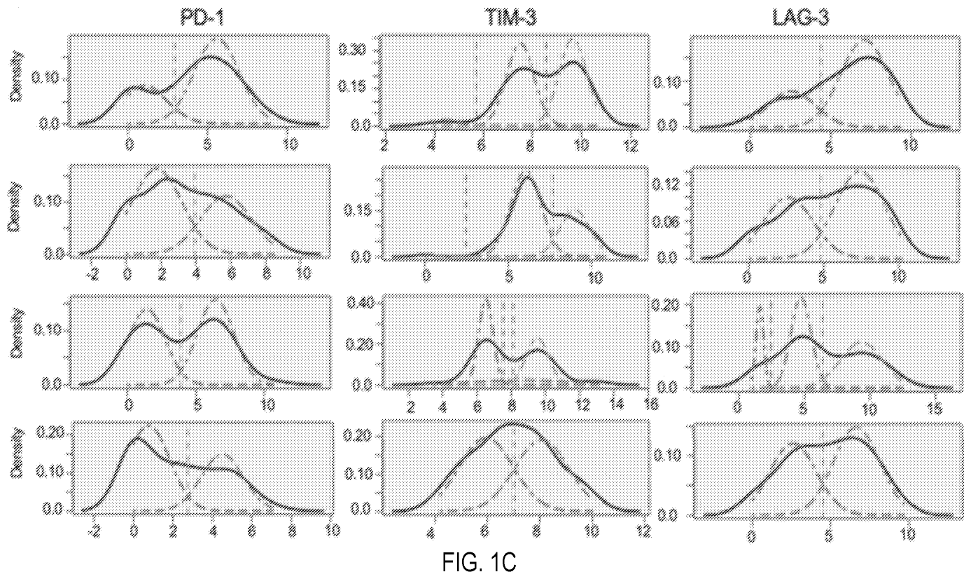

CD8 TILs have a distinct gene expression signature. The present inventors conducted a differential expression analysis between experienced CD8 TILs and activated CD8 PBL samples in the four patient cohorts. After false discovery rate (FDR) adjustment, the present inventors discovered 84 genes that were differentially expressed between the two cell types, (FDR<=0.00001) (FIG. 1A). The present inventors additionally performed Gene Set Enrichment Analysis (GSEA) on each tumor type and compared differentially expressed genes between experienced TILs and activated PBL across tumor types based on the Biological Hallmarks gene set (FIG. 1B). Pathways were selected if differential expression was significant for at least one tumor type. This analysis demonstrated that CD8 TILs have a largely similar gene expression profile across tumor types. There were, however, notable differences in some pathways, including angiogenesis, IFN-g response, heme metabolism, and IFN-α responses. A separate analysis including 7 functional gene sets was used to identify pathways displaying differential gene expression between CD8 TILs and experienced PBL common to all four tumor types (FIG. S2A). Taken together, these data indicate that CD8 TILs are more similar across tumor types than their activated PBL counterparts.

Samples are stratified by PD-1, LAG-3, and TIM-3 transcript levels. Expression of immune checkpoints is associated with T cell dysfunction in cancer [25]. Concurrent expression of the immune checkpoints PD-1, LAG-3, and TIM-3 is a marker of TIL dysfunction observed in tumors that respond poorly to CIs [24]. To identify novel genes associated with exhaustion in CD8 TILs the present inventors thus stratified samples based on expression of PD-1, LAG-3, and TIM-3 (FIG. S3A). Consistent with the present inventors' previous analysis, the present inventors found that coexpression of these checkpoints clustered samples by cell type and patient across malignancies. For example, naive cells showed low checkpoint expression compared with activated PBL and antigen-experienced TIL. Patients expressing high levels of immune checkpoints in TILs also generally expressed high levels of checkpoints in PBL and vice versa. PD-1 co-expression with TIM-3 or LAG-3 was highest in antigen-experienced TIL samples (except for PRAD, where coexpression was highest in activated PBL). Concordance of these data with previously reported patterns of immune checkpoint expression on exhausted CD8 TILs suggested that total RNA transcript levels could be used to infer relative degrees of exhaustion [24].

Based on this observation, the present inventors developed a model that dichotomized samples as expressing high levels of immune checkpoints (+) or low levels of immune checkpoints (−). To generate this model, the present inventors first evaluated the range of PD-1, LAG-3, and TIM-3 expression (FIG. S3B). This analysis suggested a bimodal distribution where naive T cells primarily cluster into the negative distribution and activated and experienced cells are overrepresented in the positive distribution. The present inventors then applied the expectation maximization (EM) algorithm to estimate two underlying distributions [26]. FIG. 1C shows the estimated underlying distributions along with the cut-points determined by the present inventors' model. All samples were then fit to this model (FIG. S4A).

Interestingly, while experienced TIL and activated PBL samples were typically in the positive distribution, this was not uniformly the case and varied with tumor type. In GBM and RCC, almost all TIL samples were positive for multiple checkpoints while several TIL samples were negative for one or more checkpoints in PRAD and BLCA (Table 1). Positive or negative status of all samples is displayed as a heatmap in FIG. S4B where samples are grouped into clusters by cell type with rows forming checkpoint genes and columns representing patients. The stratification for GBM samples is shown in a tree structure in FIG. S4C, highlighting the available subgroups. For example, all samples that were PD-1- and LAG-3− were also TIM-3- and there was no PD-1−, LAG-3−, TIM-3+sample. Tables S2 and S3 show the number of samples and percentage of samples, respectively, expressing one, two, or three checkpoints. These clusters are consistent with the concept of LAG-3 and TIM-3 as complementary checkpoints that are co-expressed with PD-1 [11].

TABLE 1
Stratification of Samples. For each gene and
tissue cohort, the counts and proportions of samples
that are PD1+/PD1−, TIM3+/TIM3−, LAG3+/LAG3−
after stratification through EM derived thresholds.

| | | Negative | Positive | | |
|---|---|---|---|---|---|
| Tissue | Gene | Samples | Samples | Negative % | Positive % |
| GBM | PD1 | 8 | 16 | 33.3 | 66.7 |
| GBM | TIM3 | 12 | 12 | 50 | 50 |
| GBM | LAG3 | 5 | 19 | 20.8 | 79.2 |
| PRAD | PD1 | 25 | 23 | 52.1 | 47.9 |
| PRAD | TIM3 | 34 | 14 | 70.8 | 29.2 |
| PRAD | LAG3 | 14 | 34 | 29.2 | 70.8 |
| RCC | PD1 | 9 | 15 | 37.5 | 62.5 |
| RCC | TIME3 | 10 | 14 | 41.7 | 58.3 |
| RCC | LAG3 | 9 | 15 | 37.5 | 62.5 |
| BLCA | PD1 | 13 | 11 | 54.2 | 45.8 |
| BLCA | TIM3 | 12 | 12 | 50 | 50 |
| BLCA | LAG3 | 8 | 16 | 33.3 | 66.7 |

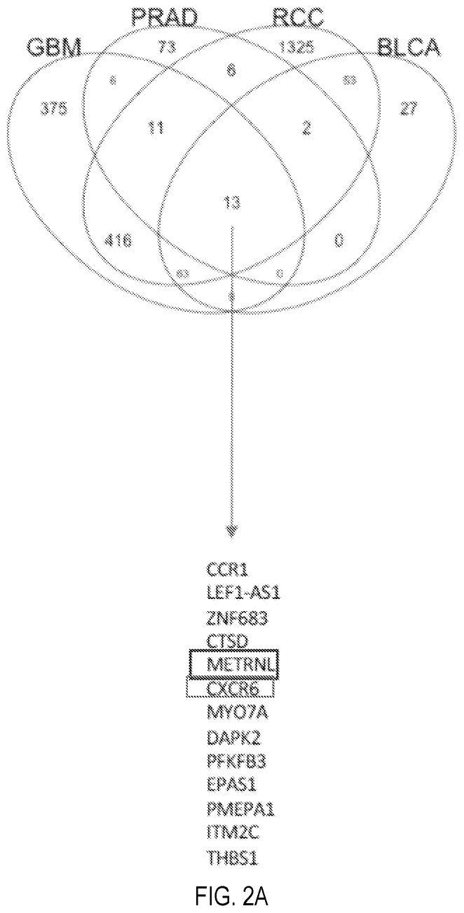

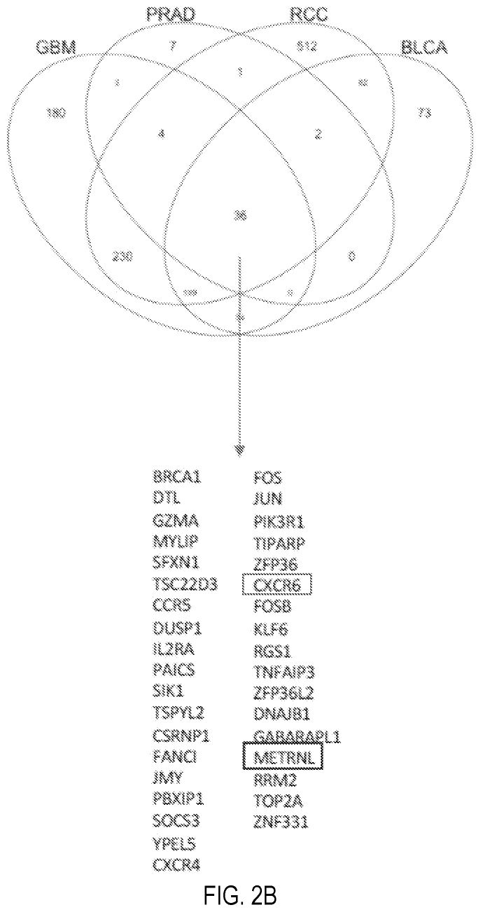

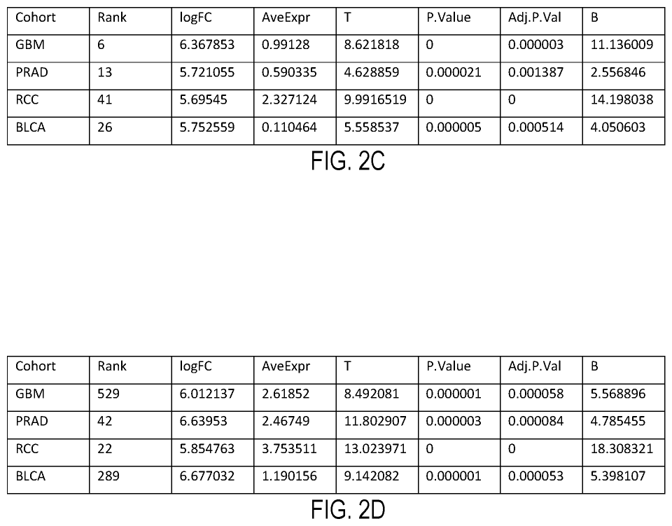

Differential gene expression identifies METRNL and CXCR6 as a marker of CD8 TIL. The present inventors performed two different analyses to identify novel genes associated with an exhausted phenotype in CD8 TIL. First, the present inventors performed a differential expression analysis for each subgroup against all other CD8 samples (e.g., PD-1+, LAG-3+, TIM-3+ against all other samples, PD-1+, LAG-3+, TIM-3− against all other samples, etc.). The present inventors were particularly interested in the contrast between triple positive (PD-1+, LAG-3+, TIM-3+) TILs against all other samples. The results of these differential expression analyses are provided as a sortable spreadsheet (Supplemental data, not shown). The false discovery rate adjustment of p-values was carried out globally for all contrasts. This analysis identified 13 genes differentially expressed by triple positive cells compared with all other samples with an FDR threshold of 0.01 (FIG. 2A).

To isolate gene expression patterns associated with intratumoral location the present inventors performed GSEA analysis of checkpoint triple positive CD8 TILs vs. triple positive PBL. Comparing this analysis with the GSEA analysis of CD8 TILs vs. all PBL revealed that intratumoral location was associated with distinct patterns of differential gene expression. Therefore, a separate differential expression analysis was performed to identify genes associated with intratumoral location among checkpoint-expressing CD8 T cells. For each tumor type the present inventors contrasted triple positive TILs against triple positive activated PBL samples, selecting patients in common for the two sample groups. The results of this analysis identified 36 differentially expressed genes (FDR<0.0001) (FIG. 2B). Combing the results of these two analyses identified 2 genes represented on both lists, CXCR6 and METRNL. FIG. 2C and FIG. 2D show the results for METRNL in the first and second analysis, respectively, with ranks being determined based on adjusted p-values. Based on the concordance of METRNL expression with immune checkpoints as well as its specificity for intratumoral location, the present inventors sought to determine if METRNL has a functional role in adaptive antitumor immunity.

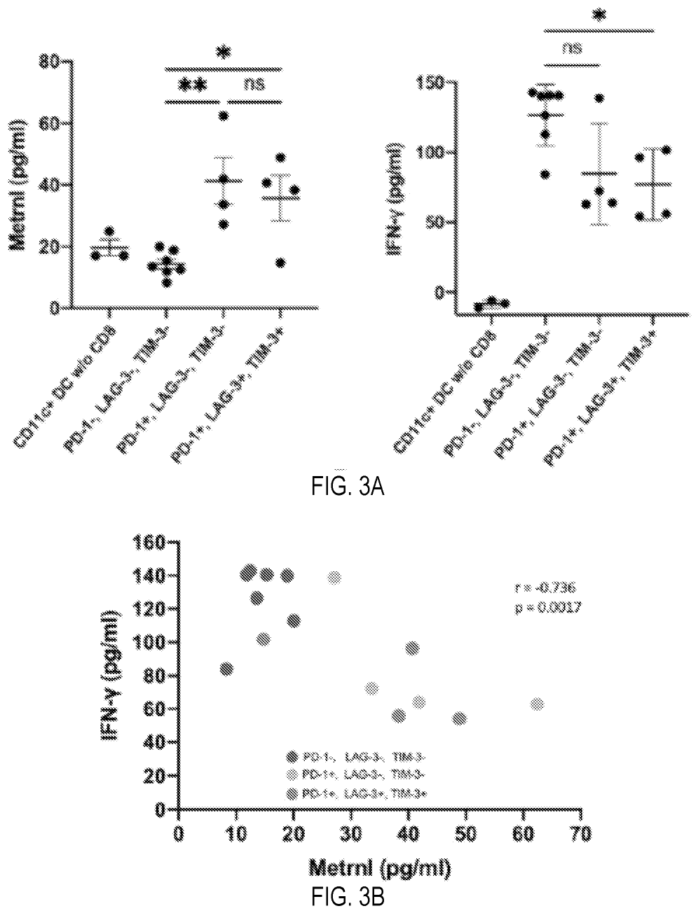

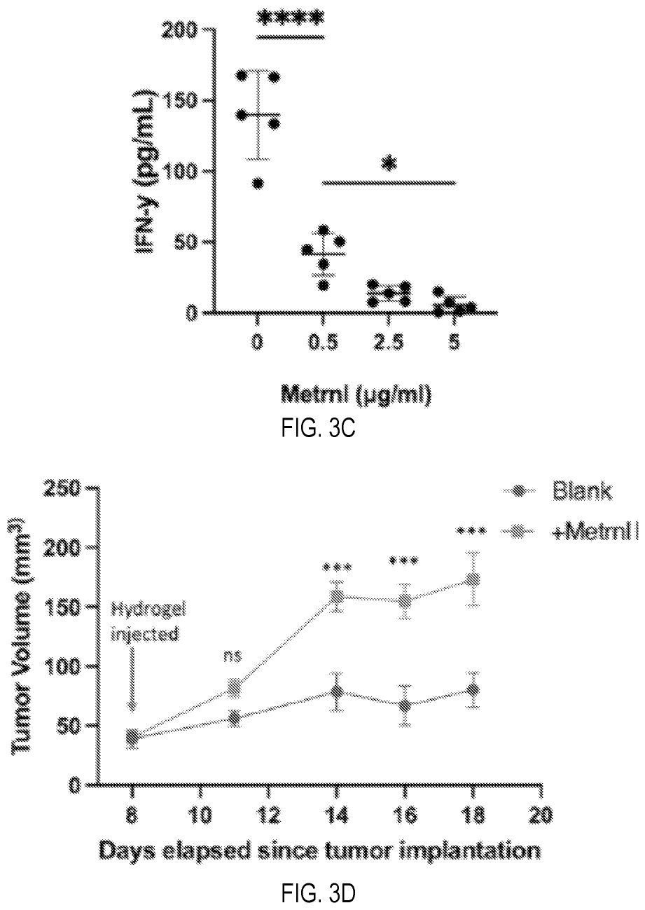

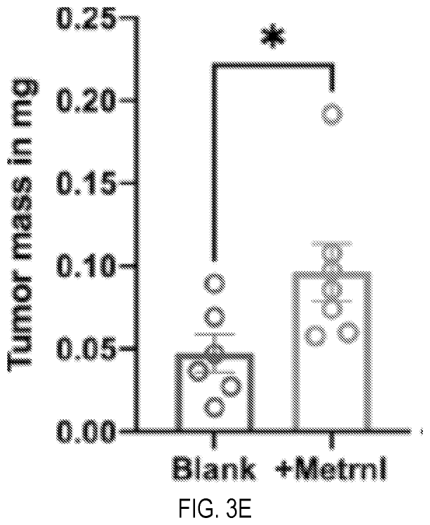

Metrnl is co-expressed with immune checkpoints in murine glioma CD8 TILs and inversely correlates with IFN-g secretion. The present inventors' comparative transcriptomics analysis revealed METRNL differential expression ranked highest for association with checkpoint expression in the GBM dataset (FIG. 2C). Therefore, the present inventors used GL261, a C57BL/6 syngeneic glioma cell line, to determine if Metrnl secretion correlates with immune checkpoint expression and impaired cytotoxicity. Mice with confirmed orthotopic GL261 tumor engraftment were sacrificed, and TILs were isolated from brain tumor tissue. CD8 TILs were sorted based on immune checkpoint expression into 3 groups: PD-1−, LAG-3−, TIM-3−; PD-1+, LAG-3−, TIM-3−; and PD-1+, LAG-3+, TIM-3+. CD8 TILs were cocultured in a 1:5 ratio with CD11c+ dendritic cells from tumor-free mice in the presence of GL261 lysate. Supernatants were collected and assayed for Metrnl and IFN-g by ELISA. Consistent with the present inventors' hypothesis that Metrnl secretion would be increased CD8 TILs with an exhausted phenotype, the present inventors observed significantly higher concentration of Metrnl in wells containing PD-1+, LAG-3+, TIM-3+ TILs compared to checkpoint-negative TILs (FIG. 3A). The checkpoint-negative TIL population isolated from murine GBM are activated effector T cells as they abundantly secreted IFN-g (FIG. 3A) and express CD44, a marker of T cell activation. Furthermore, IFN-g levels inversely correlated with Metrnl as checkpoint-positive TILs produced less IFN-g compared to activated CD8 TIL. The inverse correlation between Metrnl and IFN-g was evident on a well-by-well basis (FIG. 3B).

Metrnl suppresses CD8 T cell activation and accelerates tumor growth. To better understand the relation between Metrnl secretion and CD8 effector function, the present inventors used an in vitro assay to examine the effect of exogenous Metrnl treatment on CD8 T cell activation. CD8 T cells expressing a T cell receptor (TCR) specific for the ovalbumin epitope SIINFEKL were isolated from OT-1 transgenic mice through negative selection and plated with CD11c+ dendritic cells isolated from the spleens of tumor-naive C₅₇BL/6 mice expressing the congenic marker CD45.1 in a 1:5 ratio, along with soluble SIINFEKL peptide. Metrnl was added to the wells at increasing doses of 0.5 μg/ml, 2.5 μg/ml and 5 μg/ml. After incubating for 48 hours, supernatants were collected and assayed for IFN-g by ELISA as a proxy for activation. Metrnl suppressed IFN-g production by OT-1 T cells in a dose-dependent fashion (FIG. 3C). ELISA data were confirmed by flow cytometry for IFN-g (data not shown). These data demonstrated that METRNL not only correlates negatively with IFN-y secretion but can also directly suppress IFN-y secretion from CD8 T cells.

To further explore the immunosuppressive role of Metrnl, the present inventors injected exogenous Metrnl dissolved in hydrogel at the site of flank MC38 colorectal cancer and monitored tumor growth kinetics. Release of Metrnl at the tumor site increased tumor growth significantly compared to empty hydrogel injection (FIG. 3E, F). Exogenous Metrnl did not promote growth of tumor cells in vitro, indicating its pro-tumor effect in vivo is due to effects on non-tumor cell components of the TME (FIG. S5).

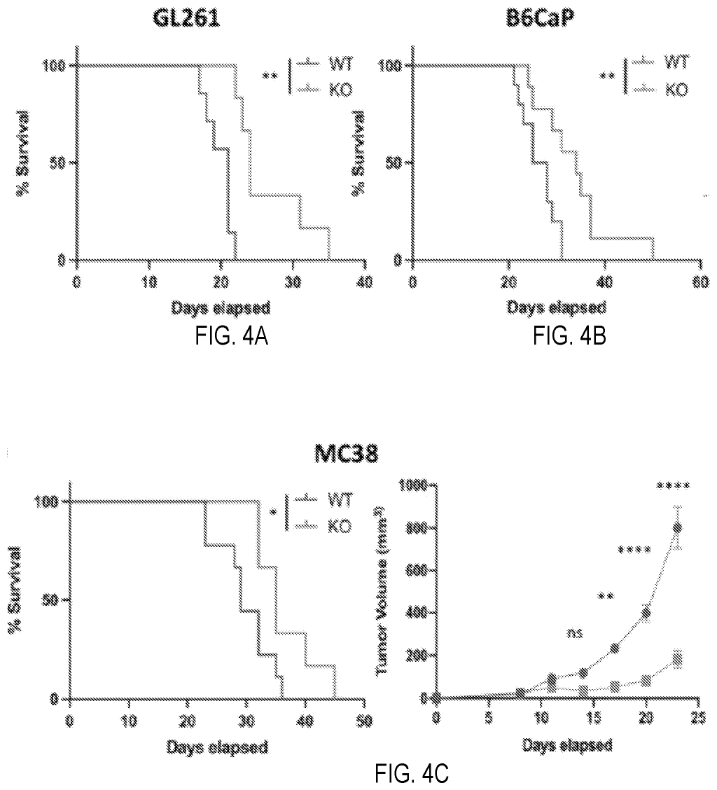

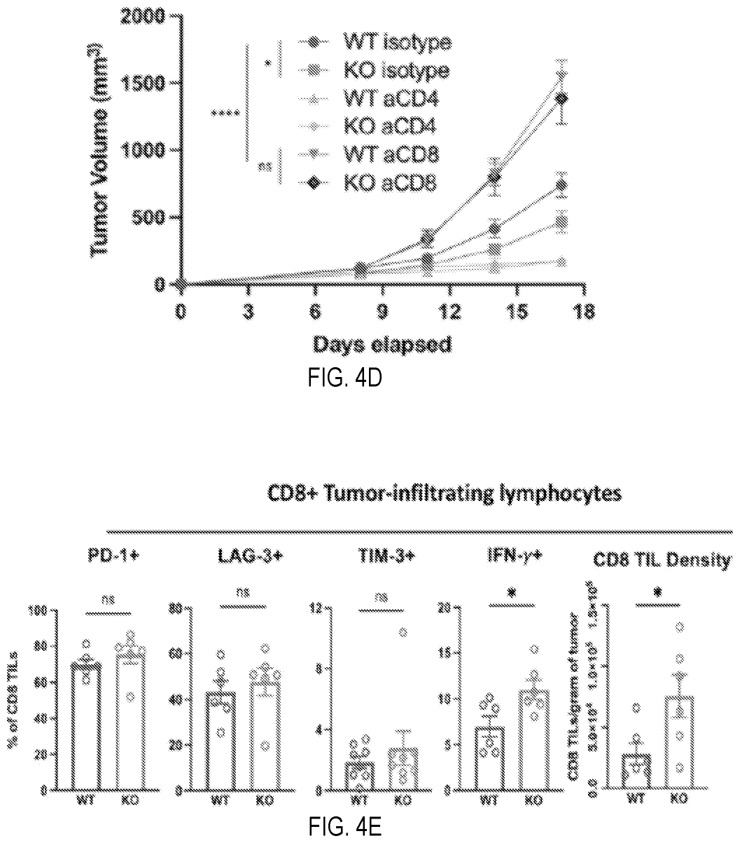

Metrnl ablation enhances anti-tumor responses in a CD8-dependent manner. The present inventors' observations indicated Metrnl is an immunosuppressive cytokine secreted by checkpoint positive TILs that can suppress CD8 T cell effector function. To determine if ablating Metrnl non-tumor tissues augments anti-tumor immunity, the present inventors compared immune response in Metrnl KO and wildtype mice bearing orthotopic glioma, flank prostate and colorectal tumors. Metrnl KO mice with GL261 glioma had improved survival compared to their wildtype cohort. (FIG. 4A). Metrnl KO mice controlled B6CaP prostate cancer and MC38 colorectal cancer growth better and exhibited enhanced survival compared to wildtypes (FIG. 4 B, C).

To determine if Metrnl ablation improves anti-tumor response in a CD8-dependent manner, the present inventors depleted CD4 and CD8 T cells in MC38 tumor-bearing wildtype and Metrnl KO mice. Depletion of CD8 T cells abrogated the effect observed in Metrnl KO mice but CD4 depletion did not, demonstrating that the effect of Metrnl ablation is CD8 dependent (FIG. 4D). Of note, CD4-depleting monoclonal antibody (GK1.5) significantly decreased tumor growth, due to depletion of CD4+ Tregs as previously reported [27]. The present inventors further investigated whether the exhaustion profile, cytotoxic activity, and viability of intratumoral CD8 T cells were improved by ablating endogenous Metrnl secretion. Flow cytometric analysis of MC38 tumors revealed CD8 TILs in Metrnl KO and wildtype mice were not different in terms of expression of immune checkpoints (FIG. 4E), suggesting that while Metrnl is co-expressed with immune checkpoints, it does not induce their expression. However, the present inventors observed a higher percentage of IFN-y+ TILs in Metrnl KO mice (FIG. 4E), indicating a robust immune response in the context of METRNL ablation. Metrnl KO mice also had higher density of CD8 TILs.

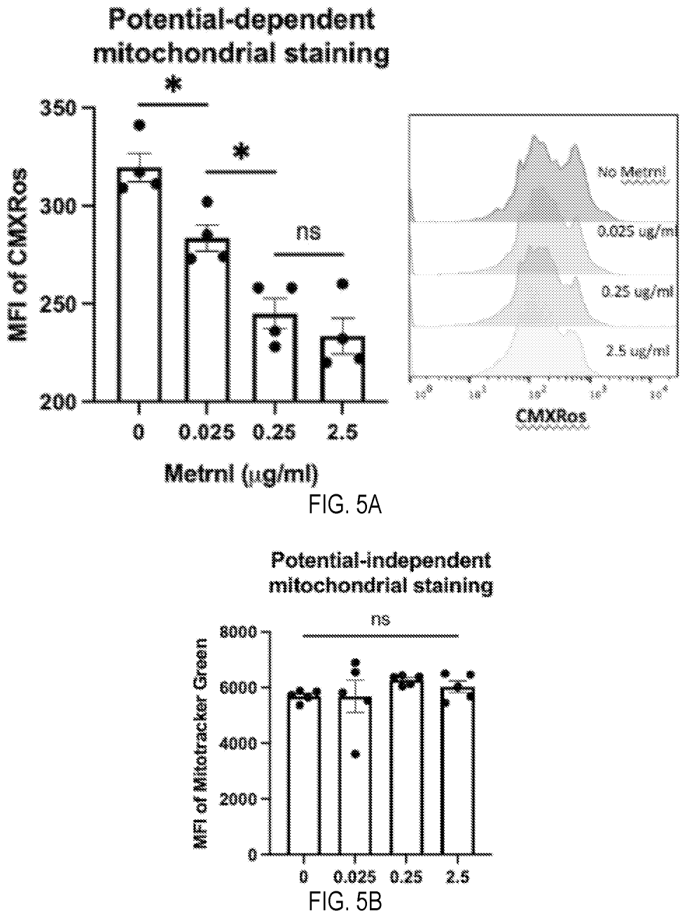

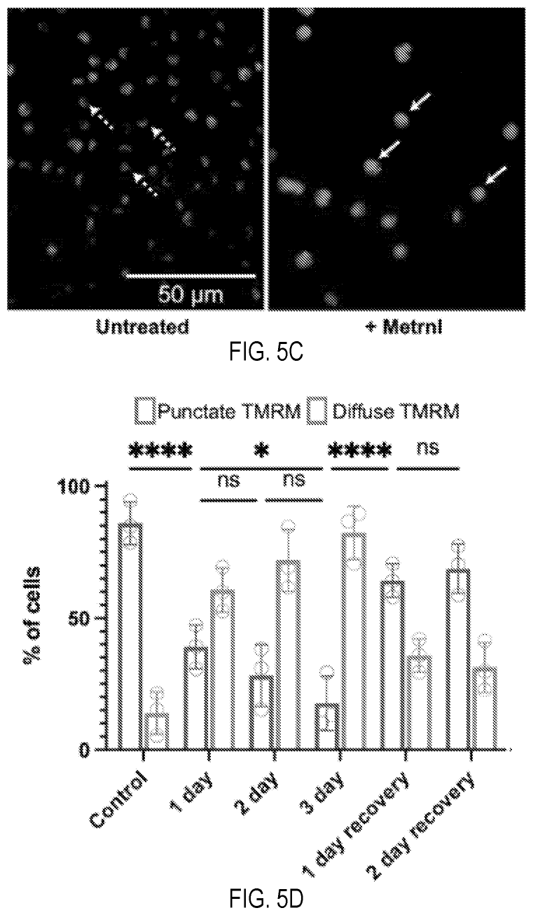

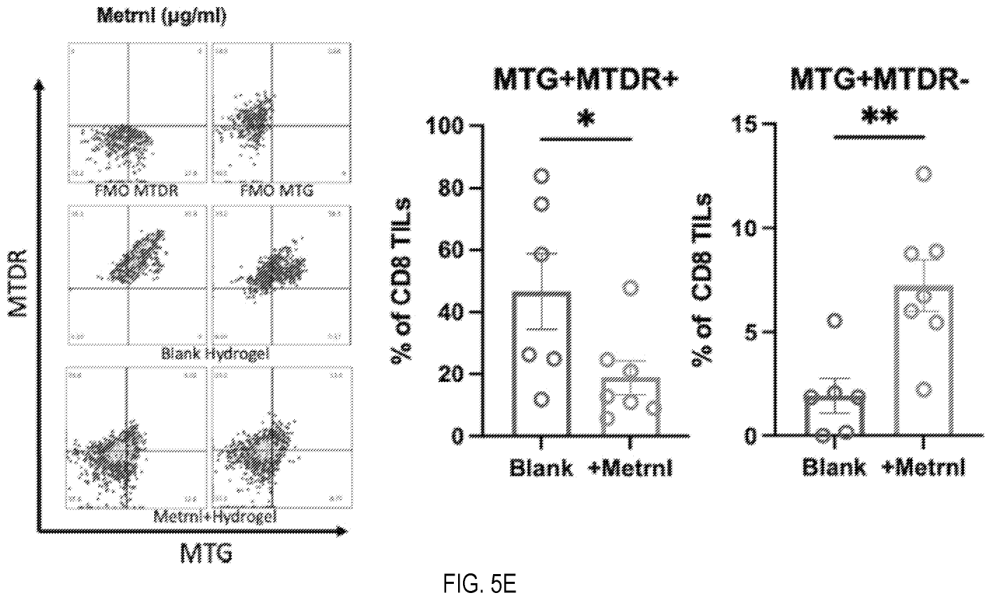

Exogenous Metrnl depolarizes CD8 T cell mitochondria. The metabolic profile of T cells is correlated with T cell fate and function [28,29] and dysregulation of metabolic processes can drive CD8 T cell exhaustion during an ongoing immune response [30]. METRNL has been shown to regulate several metabolic pathways, including improving glucose tolerance in myocytes and decoupling the mitochondrial electron transport chain in adipocytes [16]. Since Metrnl is secreted by checkpoint-positive TILs and can inhibit CD8 T cell effector phenotype, the present inventors investigated the role of Metrnl in promoting metabolic exhaustion in activated T cells. The present inventors stimulated naive T cells with phorbol-12-myristate-13-acetate (PMA) and Ionomycin for 6 hours in the presence of increasing doses of exogenous Metrnl. Using potential-independent and potential-dependent dyes, the present inventors assessed mitochondrial mass and membrane potential, respectively, in T cells. Metrnl promoted mitochondrial depolarization in a dose-dependent manner without affecting mitochondrial mass, demonstrating Metrnl's role in disrupting bioenergetic efficiency (FIG. 5A, B).

Confocal microscopy imaging of activated CD8 T in the presence of exogenous Metrnl revealed a diffuse pattern of staining with the potential-dependent mitochondrial dye Tetramethylrhodamine, methyl ester (TMRM), indicating a lack of integrity of the mitochondrial membrane. In contrast, untreated T cells displayed localization of TMRM in a punctate pattern, consistent with accumulation of the dye in intact mitochondria. (FIG. 5C). Treatment with Metrnl for 3 days resulted in the greatest percentage of cells staining diffusely with TMRM (FIG. 5D and FIG. S7). Of note, the effect of Metrnl on mitochondria was reversible. Mitochondrial localization of TMRM improved after cells were allowed to recover in the absence of exogenous Metrnl (FIG. 5D). Robust recovery of mitochondria was evident 24 hours after removing Metrnl and did not improve further after a 48-hour recovery period.

To investigate if the tumor-promoting effect of Metrnl involves metabolic suppression of CD8 TILs, the present inventors injected exogenous Metrnl dissolved in hydrogel at the site of flank MC38 tumors and assessed TILs mitochondrial mass and potential with Mitotracker Green (MTG) and Mitotracker Deep Red (MTDR), respectively. Metml injection decreased the proportion of T cells with polarized mitochondria (MTG+MTDR+), whereas T cells with depolarized mitochondria increased (MTG+MTDR−) (FIG. 5E). These findings support Metml's role in promoting mitochondrial dysfunction cascade and suggests that said dysfunction can be reversed by removing Metrnl.

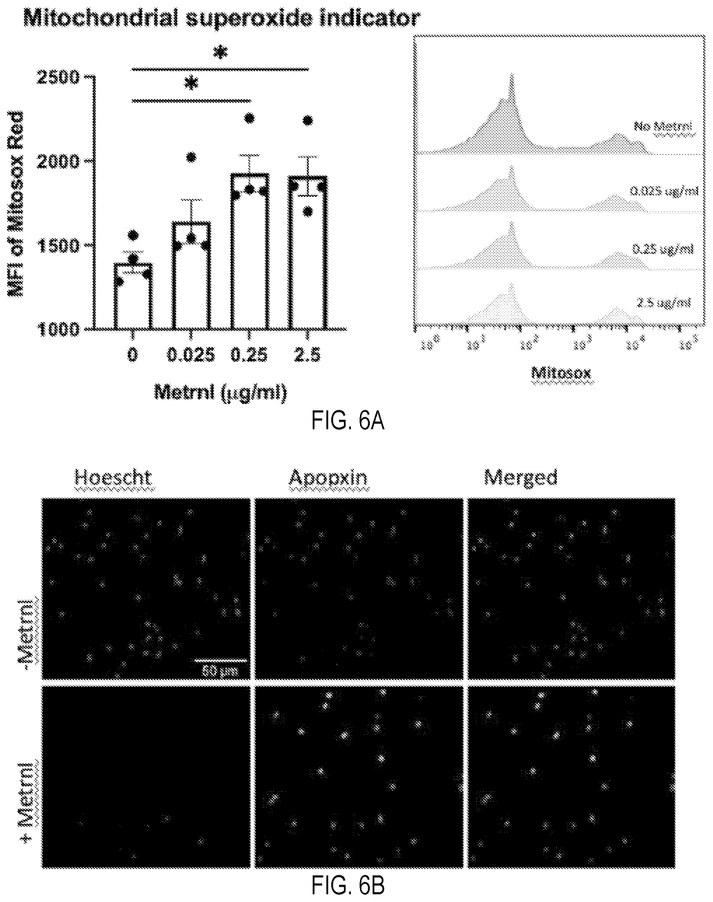

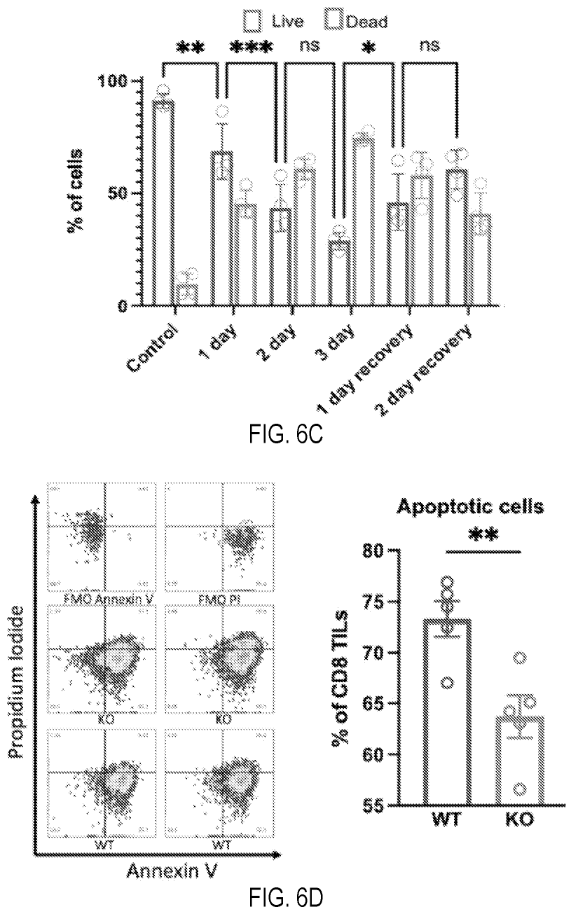

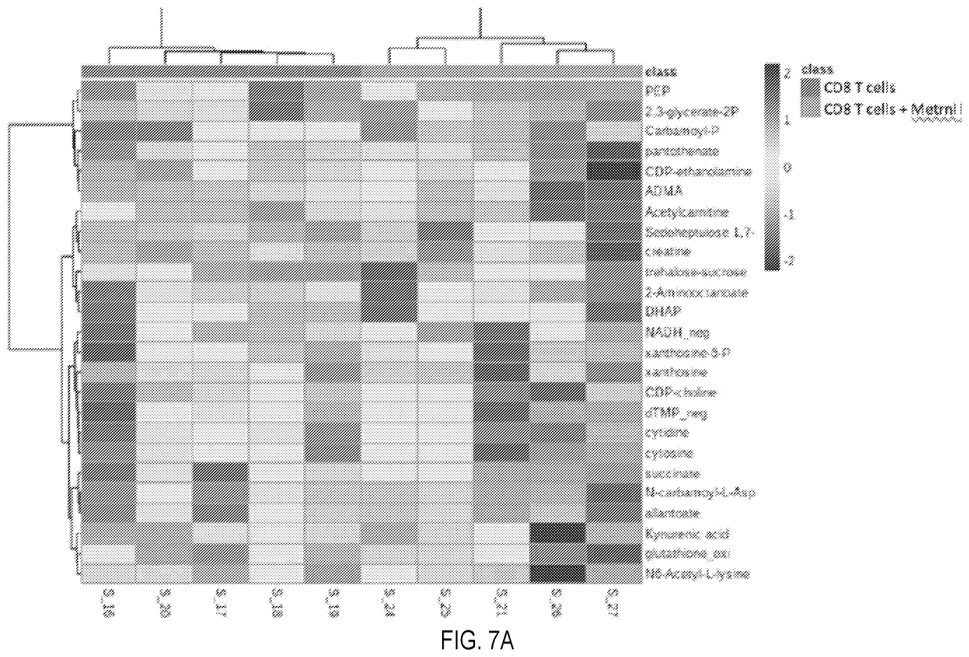

Exogenous Metrnl promotes ROS accumulation and apoptosis of CD8 T cells. Depolarization of mitochondria can increase ROS generation, making exhausted T cells susceptible to apoptosis [30]. The present inventors observed CD8 T cells treated with Metrnl exhibited a dose dependent increase in ROS accumulation in the cell (FIG. 6A). Furthermore, confocal microscopy imaging showed an increase in T cell apoptosis with Metrnl treatment (FIG. 6B). The duration of Metrnl treatment determined the extent of apoptosis, with treatment for 3 days resulting in the greatest percentage of apoptotic cells (FIG. 6C). Notably, viability of T cells also improved after cells were allowed to recover in the absence of exogenous Metrnl (FIG. 6C). Both the decline and recovery of cell viability lagged changes in mitochondria, which were more sensitive to Metrnl-induced damage and recovered more rapidly following removal of Metml (FIG. 5D).

The present inventors examined whether apoptosis of TILs in the immunosuppressive milieu of the TME would be reduced in Metrnl KO mice. Co-staining TILs with Annexin V and Propidium Iodide (PI) revealed a lower percentage of apoptotic CD8 TILs (Annexin V+, PI−) in tumors isolated from Metrnl KO mice (FIG. 6D), further supporting the present inventors' hypothesis that METRNL induces TIL apoptosis. Collectively, these findings suggest the dysregulation of cellular metabolic homeostasis promoted by METRNL limits the viability of T cells.

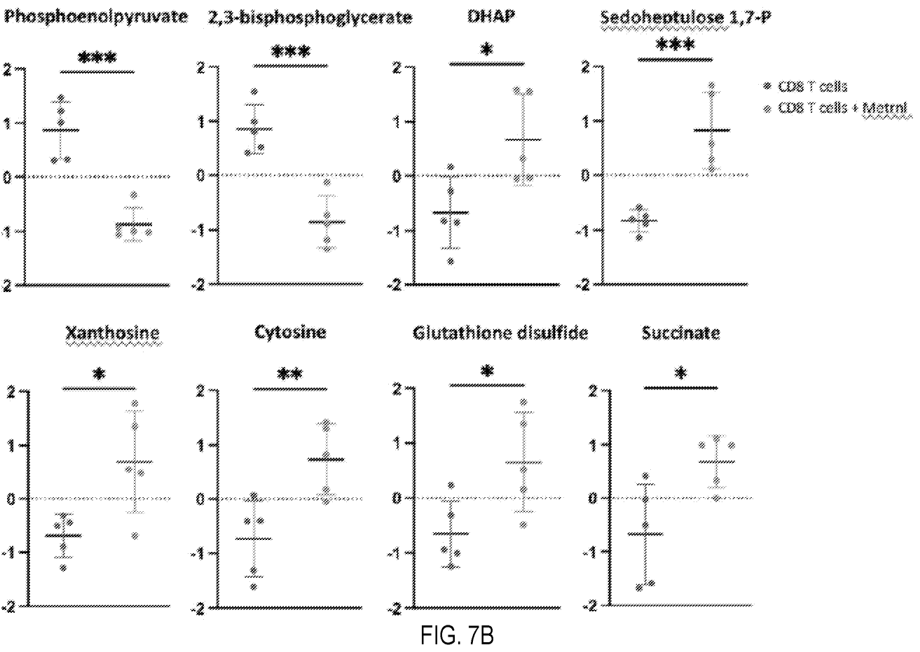

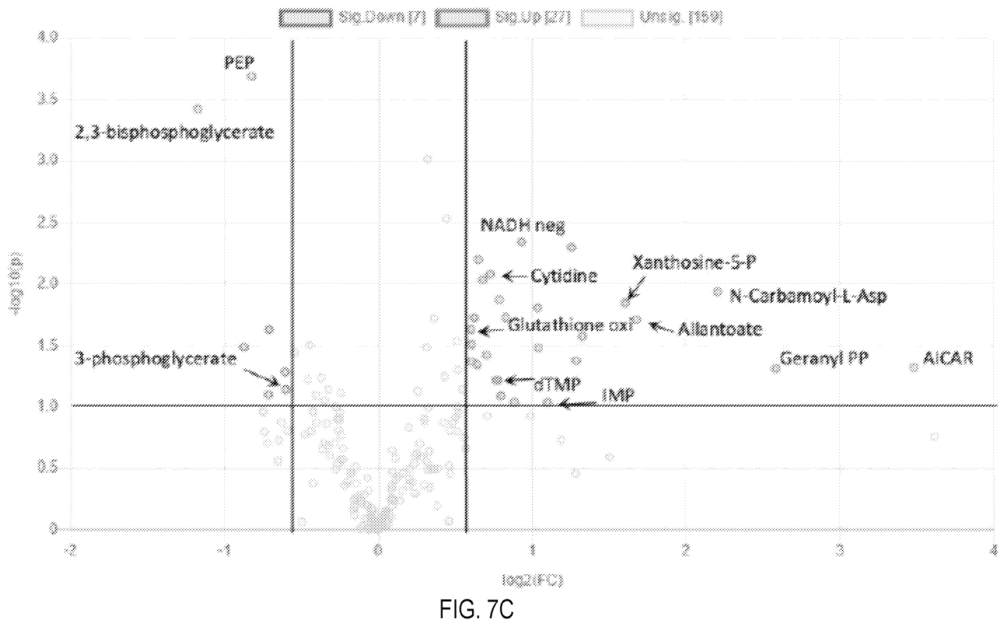

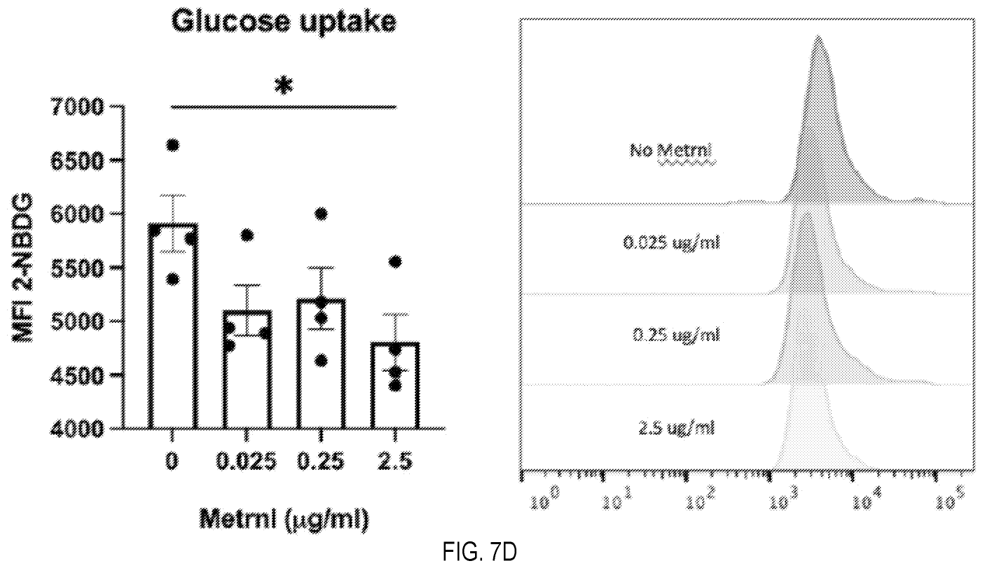

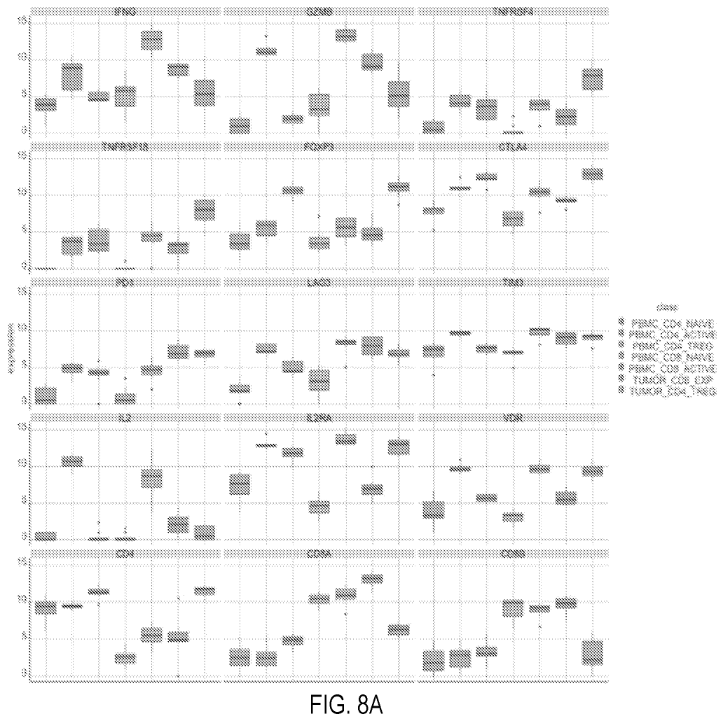

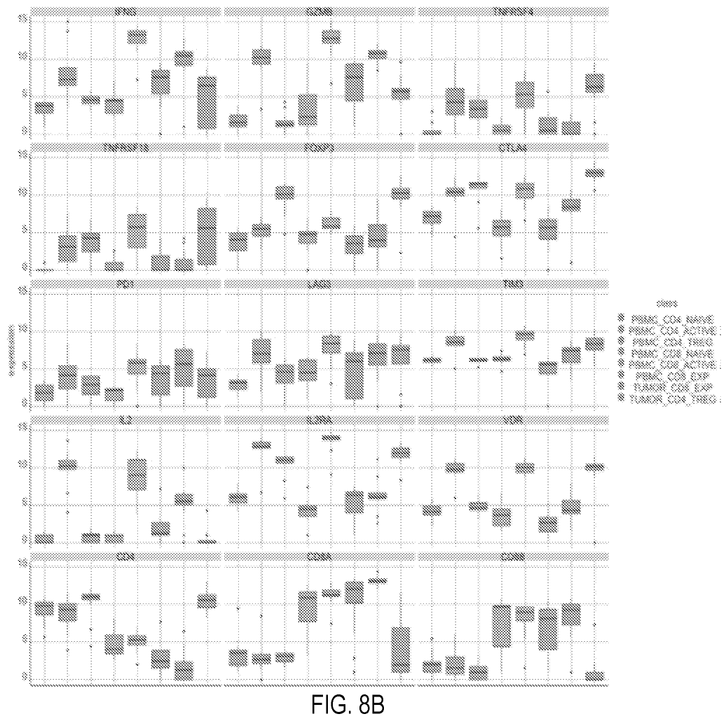

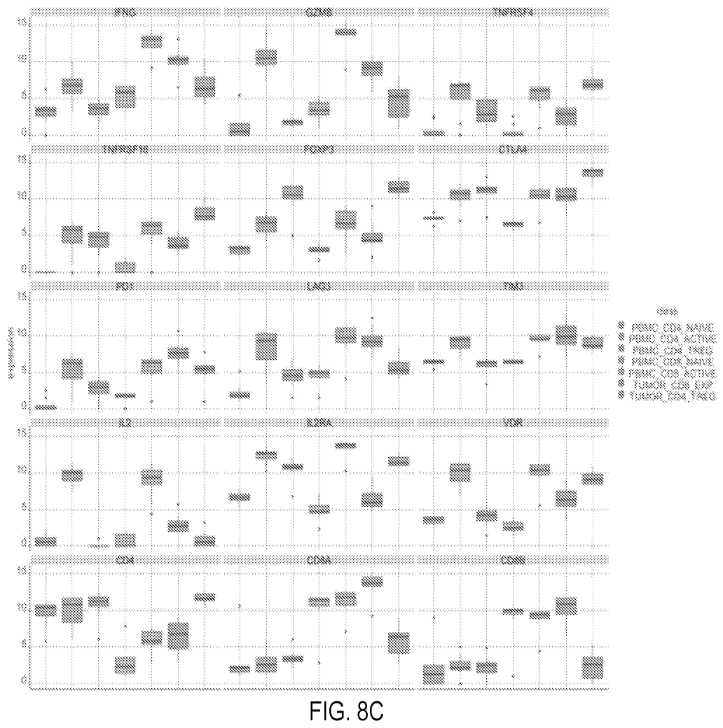

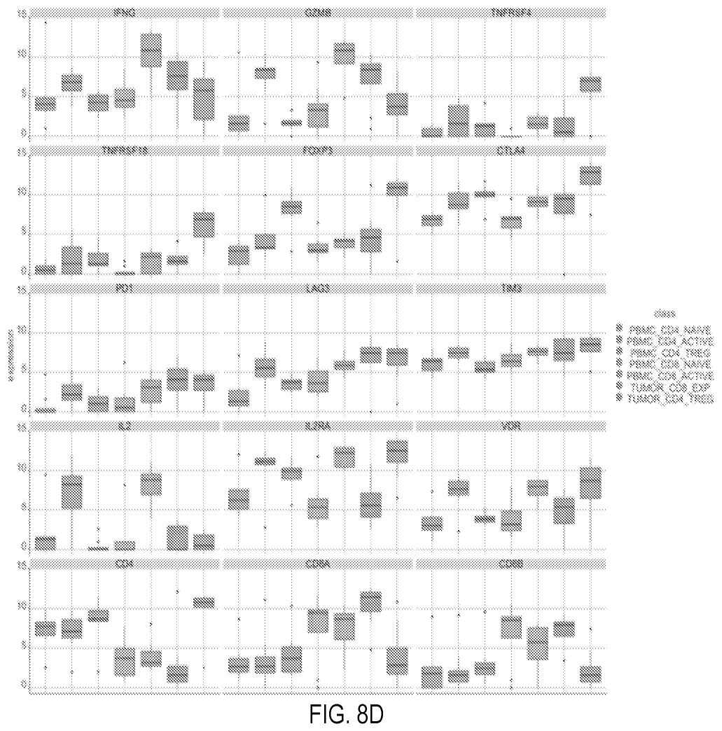

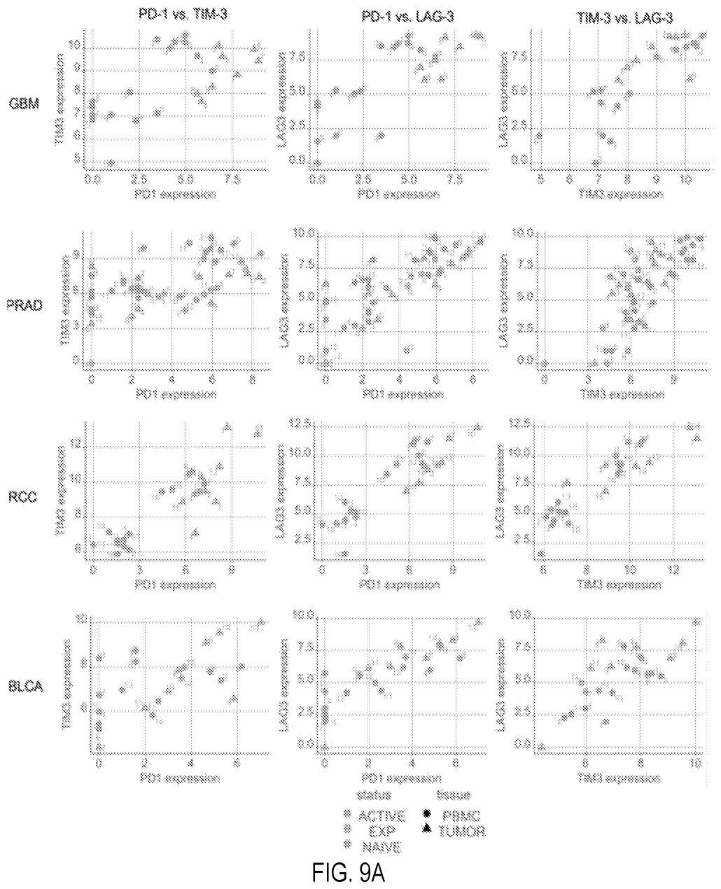

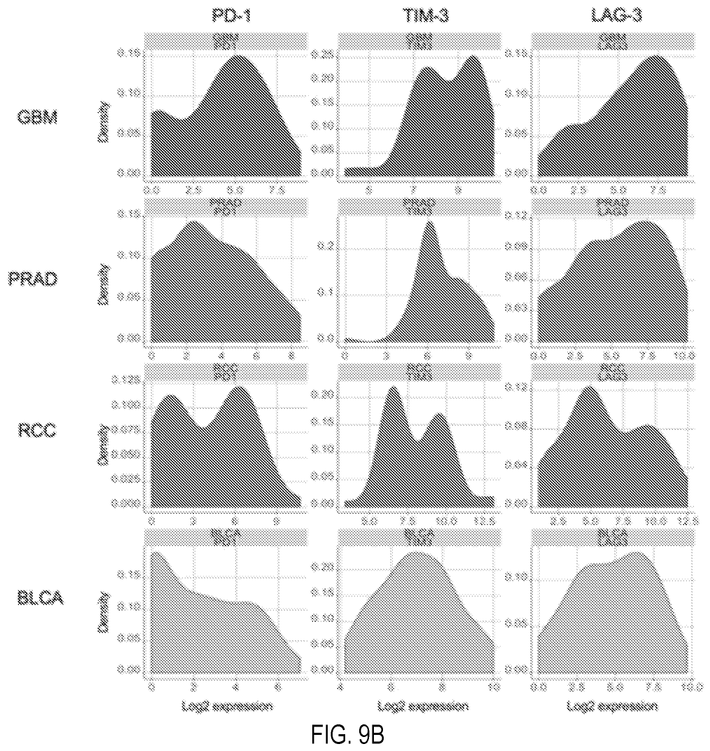

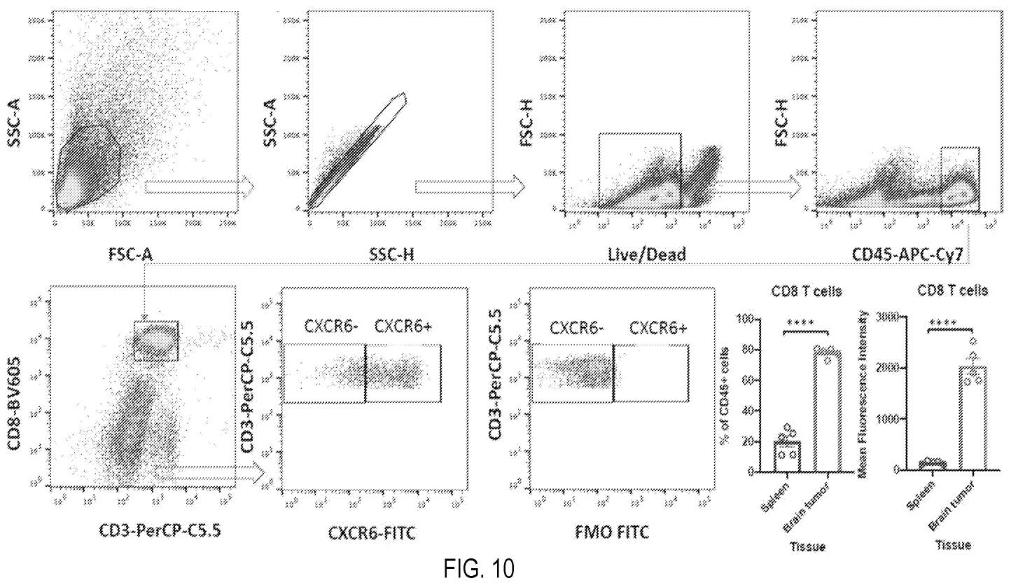

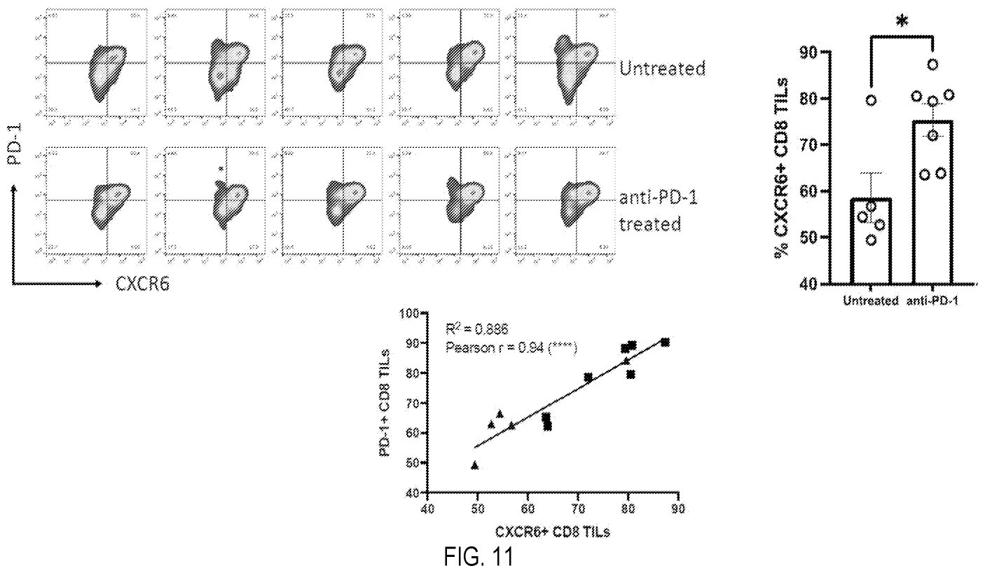

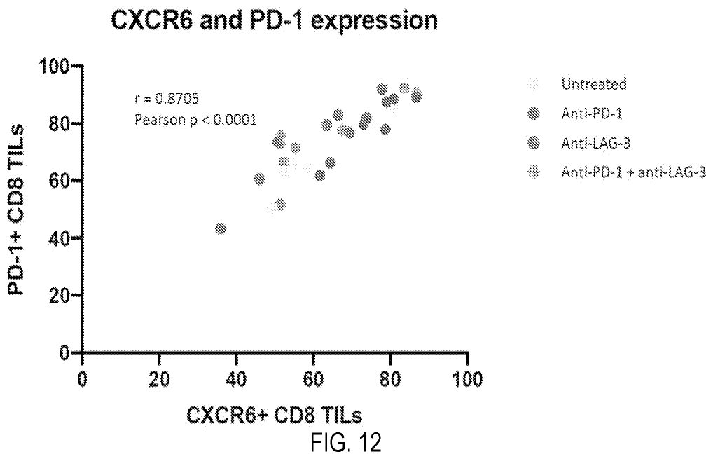

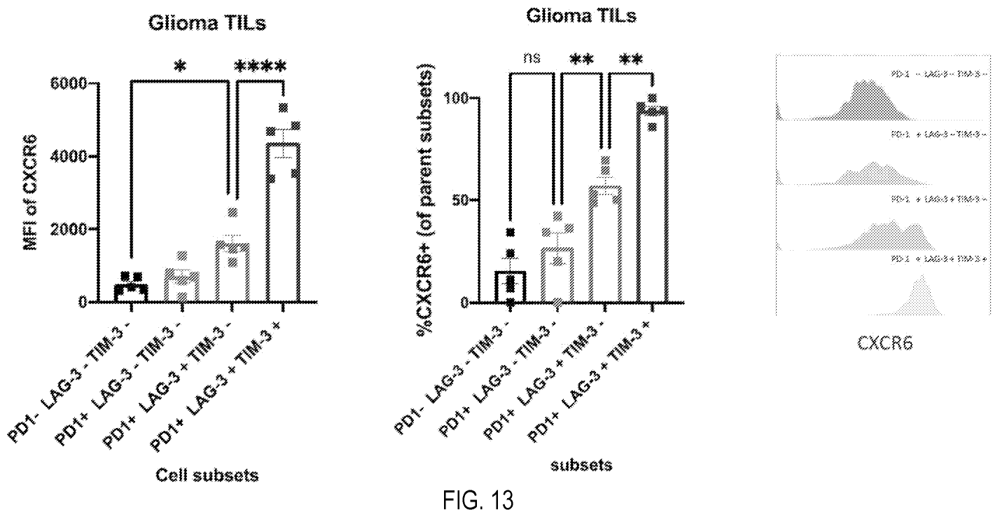

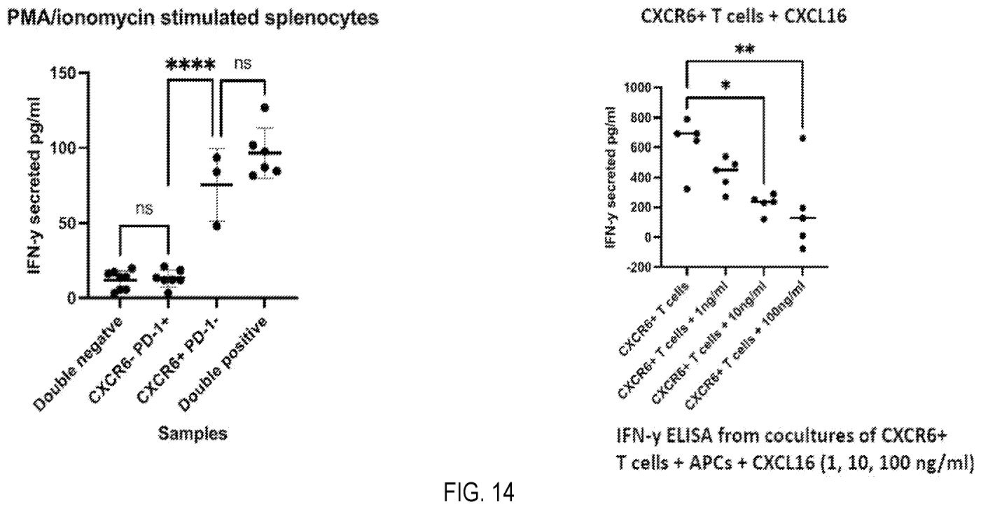

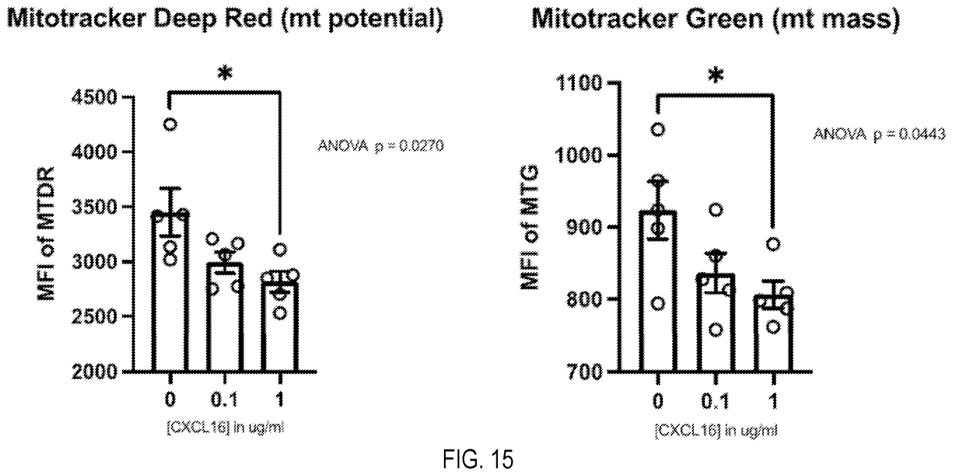

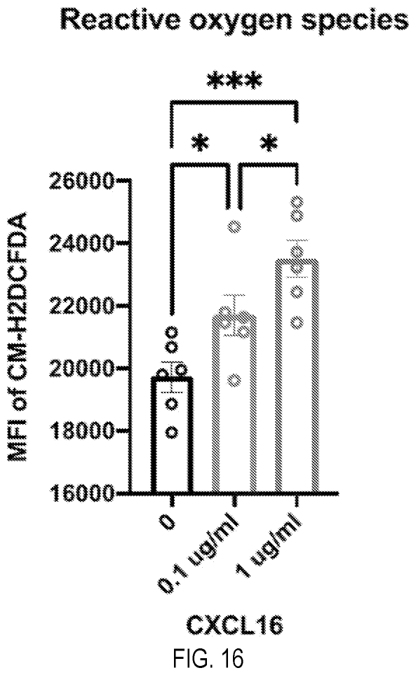

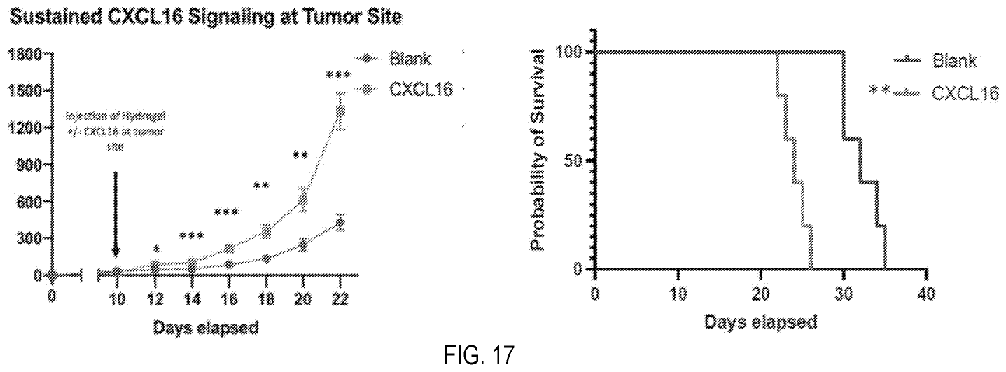

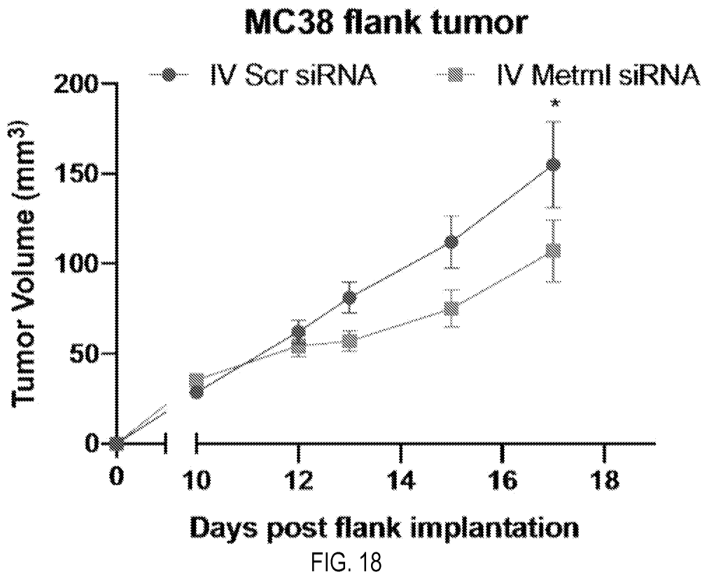

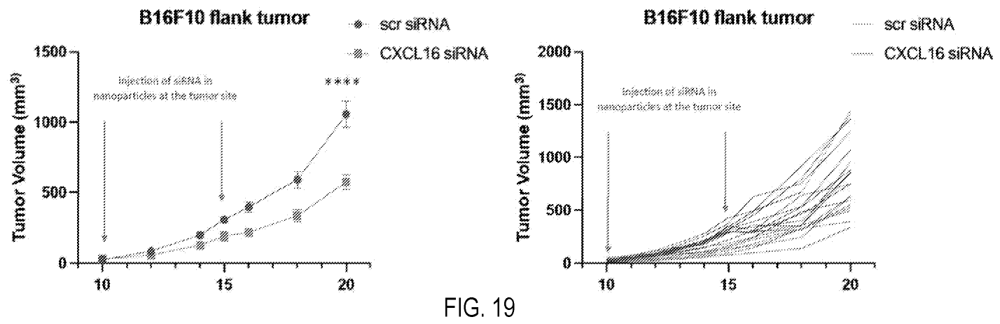

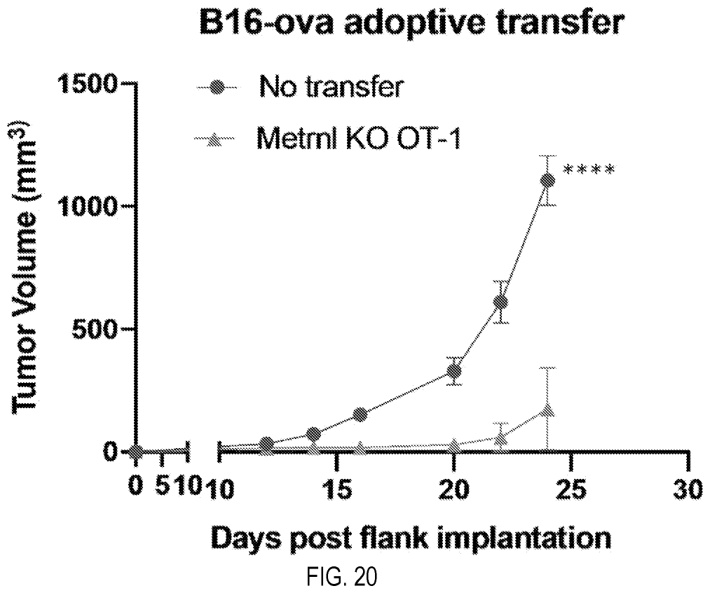

Quantitative mass spectrometry-based metabolomics analysis reveals oxidative stress and bioenergetic dysregulation with Metrnl treatment. The present inventors assessed metabolic alterations elicited by exogenous Metrnl using liquid chromatography mass spectrometry (LC-MS) analysis of metabolites in T cells. Consistent with the present inventors' in vitro experiments where treatment with Metrnl increased ROS staining in T cells, the present inventors noted metabolites indicative of oxidative stress increase in response to Metrnl (FIG. 7A-C). Oxidative stress can result in damage to DNA and cellular components [32,33] and causes metabolic flux to be redirected from glycolytic pathway to oxidative pentose phosphate pathway (PPP) and nucleotide synthesis in an attempt to reduce oxidative damage, generate reducing equivalents, and repair DNA damage [34]. Metabolites increased in Metrnl-treated T cells include Sedoheptulose 1,7-bisphosphate, a metabolite of the PPP [34,35], xanthosine, xanthosine-monophosphate cytosine, dTMP, and allantoate a product of purine catabolism that can act as a scavenger for ROS (FIG. 7A-C) [36]. Glutathione disulfide, generated during reduction of ROS by glutathione [37,38], was increased in Metrnl-treated cells, along with acetyl-L-carnitine, which is known to have both pro- and anti-oxidative roles in mammalian cells [39,40]. TCA cycle intermediate succinate, which can induce mitochondrial ROS generation, was also increased with Metml treatment [41]. Glycolytic flux in response to Metml treatment also resembled the oxidative stress response reported by Kuehne et al. involving an increase in Dihydroxyacetone phosphate (DHAP) and decrease in lower glycolysis metabolites Phosphoenolpyruvate (PEP), 1,3- and 2,3-diphosphoglycerate (FIG. 7B) [34].

To further explore changes in the glycolytic pathway, the present inventors examined whether aerobic glycolysis was suppressed in the presence of Metrnl. Based on intracellular staining with fluorescent glucose analog 2-NBDG, T cells treated with Metrnl were less efficient at glucose uptake (FIG. 7D). Glycolytic activity is crucial not only to support the metabolic needs of effector T cells but intermediates such as PEP can directly promote anti-tumor effector function of T cells [42]. The present inventors' observation that reduction of PEP levels with Metrnl treatment further supports the role of METRNL as a metabolic checkpoint.

TABLE 2
Stratification of samples. Sample counts after grouping into
single positive, double positive, and triple positive by
positive/negative status for immune checkpoint markers.

| | All | Single | Double | Triple |
|---|---|---|---|---|
| Cohort | Negative | Positive | Positive | Positive |
| GBM | 4 | 5 | 3 | 12 |
| PRAD | 13 | 11 | 12 | 12 |
| RCC | 9 | 0 | 1 | 14 |
| BLCA | 8 | 2 | 5 | 9 |

TABLE 3
Stratification of samples. Sample count percentages after grouping
into single positive, double positive, and triple positive by
positive/negative status for immune checkpoint markers.

| | All | Single | Double | Triple |
|---|---|---|---|---|
| Cohort | Negative | Positive | Positive | Positive |
| GBM | 16.67% | 20.83% | 12.5% | 50% |
| PRAD | 27.08% | 22.92% | 25% | 25% |
| RCC | 37.5% | 0% | 4.17% | 58.33% |
| BLCA | 33.33% | 8.33% | 20.83% | 37.5% |

### Discussion

CD8 T cell exhaustion limits immunity against solid tumors as density and quality of CD8 TILs correlate with clinical outcomes [43]. Immune checkpoints are markers of T cell exhaustion and have been targeted with blocking antibodies; however, CIs fail in most patients [44]. One hypothesis for CI failure is that TILs enter a state of severe, irreversible exhaustion characterized by specific genetic and epigenetic programs [45-48]. In this study, the present inventors used a comparative transcriptomics approach to identify differential gene expression patterns in sorted CD8 TILs and matched PBLs from patients with GBM, prostate cancer, RCC, and bladder cancer to identify differentially regulated genes associated with intratumoral location and exhaustion makers across tumor types. This screen identified METRNL and CXCR6 as candidate targets. Of note, CXCR6 was recently validated in a separate study as the most highly expressed chemokine receptor in TILs, and was reported to be critical for cytotoxic cell-mediated tumor control [49]. These results further support the validity of the present inventors' screen. Here the present inventors report that METRNL functions as a reversible metabolic checkpoint in TILs.

Single-cell RNA sequencing studies have highlighted TIL heterogeneity in several tumor types [50-52]. To identify common genetic threads, the present inventors reasoned that CD8 TIL-specific genes could be identified from bulk RNA sequencing data of sorted CD8 T cells by comparing differential gene expression of CD8 TILs and PBLs. To discover novel genes associated with an exhausted immunophenotype, the present inventors further hypothesized that transcript levels of PD-1, LAG-3, and TIM-3 in each sample could be leveraged to infer relative degrees of exhaustion among samples. Based on this assumption the present inventors developed a model to classify samples as “positive (+)” or “negative (−)” for checkpoint molecules using an expectation maximization algorithm. As expected, CD8 TIL samples were typically positive for multiple immune checkpoints, while naive PBL samples were negative. It is also notable that GBM TILs had the highest overall expression of one or more checkpoints, consistent with previous reports [24].

The present inventors employed the model to identify novel targets co-expressed with immune checkpoints across tumor types. This analysis identified 13 genes of interest. These genes have diverse functions, including cell migration/adhesion (THBS1, CCR1, CXCR6), apoptosis (DAPK2), lysosomal function (CTSD), and metabolism (METRNL, EPAS1, PFKFB3). The present inventors sought to further refine the screen by identifying genes correlated with intratumoral location. This analysis yielded 36 differentially expressed genes. METRNL was present on both lists, so the present inventors hypothesized that METRNL is a specific TIL exhaustion marker based on co-expression with immune checkpoints and independent association with intratumoral location.

Flow cytometric analysis of CD8 TILs isolated from murine gliomas corroborated the present inventors' human RNA data as most cells expressed no checkpoints, PD-1 alone, or all three checkpoints, with few cells expressing LAG-3 or TIM-3 in the absence of PD-1. Data from GBM indicates that PD-1 is a marker of exhaustion in TILs and a marker of activation in PBLs [53]. In a murine glioma model, exhausted CD8 TILs expressing PD-1 secreted more Metrnl than effector TILs when cultured with glioma antigens, confirming the association of Metml and checkpoint expression. In vitro activation assays revealed that Metrnl inhibits effector CD8 T cells in a dose-dependent manner and does not induce expression of immune checkpoints. Metrnl KO mice had delayed tumor growth and improved survival compared with WT animals while locally administered Metrnl accelerated tumor growth. Taken together, these findings confirm that METRNL is a novel immune regulator of TILs that co-localizes with, but does not induce, immune checkpoint expression. The latter finding raises the possibility that therapeutics targeting METRNL could have activity in CI-insensitive cancers.

METRNL is a metabolically active cytokine in skeletal muscle [20,31], adipocytes [14], and cardiac myocytes [54]. There is emerging rationale for targeting metabolic pathways to reverse immunosuppression in cancer [55,56], but few targets have been validated. Accumulation of ROS secondary to mitochondrial depolarization has been reported as a terminal mechanism of cell exhaustion [30,57,58]. The signals that govern this process, however, have not been previously identified. Mechanistically the present inventors found that Metml causes oxidative stress and mitochondrial membrane depolarization in CD8 TIL, which impaired effector function and survival in the tumor microenvironment. A quantitative approach to measure metabolite changes further revealed the extent to which Metrnl alters bioenergetic processes. CD8 T cells engaged in a compensatory response to Metrnl-induced oxidative stress by increasing antioxidant responses and nucleotide synthesis. This response has previously been reported to be counterproductive in T cell function due to blunting of the glycolytic pathway [42]. Taken together, these findings strongly implicate METRNL as a mediator of metabolic exhaustion in CD8 TIL.

The reversibility of T cell exhaustion is a topic of intense investigation and some debate. CD8 T cells under chronic antigen stimulation are subject to transcriptional regulation by TCF-1, T-bet, and Tox as they transition from effectors to reversibly exhausted and finally a terminally exhausted phenotype [45,59]. TCF-1 is a specific maker for a T cell's capacity to regain effector function in response to PD-1 blockade and loss of TCF-1 signifies transition to a terminally exhausted state [60]. Some authors propose that mitochondrial dysfunction is a pivotal step towards terminal exhaustion [57,61]. The present inventors found that mitochondrial polarization was restored, and apoptotic pathways were reversed, when Metrnl was removed, suggesting that there is a window of opportunity to rescue exhausted T cells with METRNL ablation. These data lend credence to the notion that metabolic exhaustion is not always a terminal phenomenon.

The present inventors' data identify METRNL as a metabolic checkpoint upregulated in CD8 TILs across immunologically diverse cancers. Future experiments will determine if pharmacologic inhibition of METRNL or its binding partners can reverse CD8 TIL exhaustion and mediate clinical tumor regression. More broadly, the identification of METRNL as the lead target of an agnostic screen of carefully sorted TILs and matched PBLs across otherwise immunologically diverse malignancies strongly supports metabolic dysfunction a unifying mechanism of immunosuppression in cancer.

### REFERENCES

1. Topalian S L, Drake C G, Pardoll D M. Immune checkpoint blockade: a common denominator approach to cancer therapy. Cancer Cell 2015; 27:450-461.
2. Topalian S L, Hodi F S, Brahmer J R, et al. Safety, activity, and immune correlates of anti-PD-1 antibody in cancer. N Engl J Med 2012; 366:2443-2454.
3. Borghaei H, Paz-Ares L, Horn L, et al. Nivolumab versus Docetaxel in Advanced Nonsquamous Non-Small-Cell Lung Cancer. N Engl J Med 2015; 373:1627-1639.
4. Motzer R J, Escudier B, McDermott D F, et al. Nivolumab versus Everolimus in Advanced Renal-Cell Carcinoma. N Engl J Med 2015; 373:1803-1813.
5. Sharma P, Retz M, Siefker-Radtke A, et al. Nivolumab in metastatic urothelial carcinoma after platinum therapy (CheckMate 275): a multicentre, single-arm, phase 2 trial. Lancet Oncol 2017; 18:312-322.
6. Johanns T M, Miller C A, Dorward I G, et al. Immunogenomics of Hypermutated Glioblastoma: A Patient with Germline POLE Deficiency Treated with Checkpoint Blockade Immunotherapy. Cancer Discov 2016; 6:1230-1236.
7. Boudadi K, Suzman D L, Anagnostou V, et al. Ipilimumab plus nivolumab and DNA-repair defects in A R-V7-expressing metastatic prostate cancer. Oncotarget 2018; 9:28561-28571.
8. Omuro A, Vlahovic G, Lim M, et al. Nivolumab with or without ipilimumab in patients with recurrent glioblastoma: results from exploratory phase I cohorts of CheckMate 143. Neuro Oncol 2018; 20:674-686.
9. Zhao J, Chen A X, Gartrell R D, et al. Immune and genomic correlates of response to anti-PD-1 immunotherapy in glioblastoma. Nat Med 2019; 25:462-469.

Sharma P, Hu-Lieskovan S, Wargo J A, Ribas A. Primary, Adaptive, and Acquired Resistance to Cancer Immunotherapy. Cell 2017; 168:707-723.
11. Koyama S, Akbay E A, Li Y Y, et al. Adaptive resistance to therapeutic PD-1 blockade is associated with upregulation of alternative immune checkpoints. Nat Commun 2016; 7:10501.
12. Obradovic A Z, Dallos M C, Zahurak M L, et al. T-Cell Infiltration and Adaptive Treg Resistance in Response to Androgen Deprivation With or Without Vaccination in Localized Prostate Cancer. Clin Cancer Res 2020; 26:3182-3192.
13. Anagnostou V, Smith K N, Forde P M, et al. Evolution of Neoantigen Landscape during Immune Checkpoint Blockade in Non-Small Cell Lung Cancer. Cancer Discov 2017; 7:264-276.
14. Zheng S-L, Li Z-Y, Song J, Liu J-M, Miao C-Y. Metrnl: a secreted protein with new emerging functions. Acta Pharmacol Sin 2016; 37:571-579.
15. Li Z-Y, Zheng S-L, Wang P, et al. Subfatin is a novel adipokine and unlike Meteorin in adipose and brain expression. CNS Neurosci Ther 2014; 20:344-354.
16. Rao R R, Long J Z, White J P, et al. Meteorin-like is a hormone that regulates immune-adipose interactions to increase beige fat thermogenesis. Cell 2014; 157:1279-1291.
17. Jung T W, Lee S H, Kim H-C, et al. METRNL attenuates lipid-induced inflammation and insulin resistance via AMPK or PPAR6-dependent pathways in skeletal muscle of mice. Exp Mol Med 2018; 50:122.
18. Ushach I, Burkhardt A M, Martinez C, et al. METEORIN-LIKE is a cytokine associated with barrier tissues and alternatively activated macrophages. Clin Immunol 2015; 156:119-127.
19. Ushach I, Arrevillaga-Boni G, Heller G N, et al. Meteorin-like/Meteorin-I3 Is a Novel Immunoregulatory Cytokine Associated with Inflammation. J Immunol 2018; 201:3669-3676.
20. Baht G S, Bareja A, Lee D E, et al. Meteorin-like facilitates skeletal muscle repair through a Stat3/IGF-1 mechanism. Nat Metab 2020; 2:278-289.
21. Shivakumar M, Lee Y, Bang L, Garg T, Sohn K-A, Kim D. Identification of epigenetic interactions between miRNA and DNA methylation associated with gene expression as potential prognostic markers in bladder cancer. BMC Med Genomics 2017;
22. Manning A A, Zhao L, Zhu Z, et al. IL-39 acts as a friend to pancreatic cancer. Med Oncol 2018; 36:12.
23. Szabo P A, Levitin H M, Miron M, et al. Single-cell transcriptomics of human T cells reveals tissue and activation signatures in health and disease. Nat Commun 2019;
24. Woroniecka K, Chongsathidkiet P, Rhodin K, et al. T-Cell Exhaustion Signatures Vary with Tumor Type and Are Severe in Glioblastoma. Clin Cancer Res 2018; 24:4175-4186.

Pardoll D M. The blockade of immune checkpoints in cancer immunotherapy. Nat Rev Cancer 2012; 12:252-264.
26. Do C B, Batzoglou S. What is the expectation maximization algorithm? Nat Biotechnol 2008; 26:897-899.
27. Yi H, Zhen Y, Zeng C, Zhang L, Zhao Y. Depleting anti-CD4 monoclonal antibody (GK1.5) treatment: influence on regulatory CD4+CD25+Foxp3+ T cells in mice. Transplantation 2008; 85:1167-1174.
28. Roy S, Rizvi Z A, Awasthi A. Metabolic Checkpoints in Differentiation of Helper T Cells in Tissue Inflammation. Front Immunol 2018; 9:3036.
29. Pearce E L, Poffenberger M C, Chang C-H, Jones R G. Fueling immunity: insights into metabolism and lymphocyte function. Science 2013; 342:1242454.
30. Bengsch B, Johnson A L, Kurachi M, et al. Bioenergetic Insufficiencies Due to Metabolic Alterations Regulated by the Inhibitory Receptor PD-1 Are an Early Driver of CD8(+) T Cell Exhaustion. Immunity 2016; 45:358-373.
31. Lee J O, Byun W S, Kang M J, et al. The myokine meteorin-like (metrnl) improves glucose tolerance in both skeletal muscle cells and mice by targeting AMPKa2. FEBS J 2020; 287:2087-2104.
32. Guo C, Sun L, Chen X, Zhang D. Oxidative stress, mitochondrial damage and neurodegenerative diseases. Neural Regen Res 2013; 8:2003-2014.
33. Imlay J A, Linn S. DNA damage and oxygen radical toxicity. Science 1988; 240:1302-1309.
34. Kuehne A, Emmert H, Soehle J, et al. Acute Activation of Oxidative Pentose Phosphate Pathway as First-Line Response to Oxidative Stress in Human Skin Cells. Mol Cell 2015; 59:359-371.

Cheng M-L, Lin J-F, Huang C-Y, et al. Sedoheptulose-1,7-bisphospate Accumulation and Metabolic Anomalies in Hepatoma Cells Exposed to Oxidative Stress. Oxid Med Cell Longev 2019; 2019:5913635.
36. Brychkova G, Alikulov Z, Fluhr R, Sagi M. A critical role for ureides in dark and senescence-induced purine remobilization is unmasked in the Atxdh1 Arabidopsis mutant. Plant J 2008; 54:496-509.
37. O'Donovan D J, Fernandes C J. Mitochondrial glutathione and oxidative stress: implications for pulmonary oxygen toxicity in premature infants. Mol Genet Metab 2000; 71:352-358.
38. Chakravarthi S, Jessop C E, Bulleid N J. The role of glutathione in disulphide bond formation and endoplasmic-reticulum-generated oxidative stress. EMBO Rep 2006; 7:271-275.
39. Palacios H H, Yendluri B B, Parvathaneni K, et al. Mitochondrion-specific antioxidants as drug treatments for Alzheimer disease. CNS Neurol Disord Drug Targets 2011; 10:149-162.
40. Robinson B, Gu Q, Ali S F, Dumas M, Kanungo J. Ketamine-induced attenuation of reactive oxygen species in zebrafish is prevented by acetyl 1-carnitine in vivo. Neurosci Lett 2019; 706:36-42.
41. Zhang Y, Zhang M, Zhu W, et al. Succinate accumulation induces mitochondrial reactive oxygen species generation and promotes status epilepticus in the kainic acid rat model. Redox Biol 2020; 28:101365.
42. Ho P-C, Bihuniak J D, Macintyre A N, et al. Phosphoenolpyruvate Is a Metabolic Checkpoint of Anti-tumor T Cell Responses. Cell 2015; 162:1217-1228.
43. Galon J, Angell H K, Bedognetti D, Marincola F M. The continuum of cancer immunosurveillance: prognostic, predictive, and mechanistic signatures. Immunity 2013; 39:11-26.
44. Lim M, Xia Y, Bettegowda C, Weller M. Current state of immunotherapy for glioblastoma. Nat Rev Clin Oncol 2018; 15:422-442.
45. Beltra J-C, Manne S, Abdel-Hakeem M S, et al. Developmental Relationships of Four Exhausted CD8+ T Cell Subsets Reveals Underlying Transcriptional and Epigenetic Landscape Control Mechanisms. Immunity 2020; 52:825-841.e8.
46. Bengsch B, Ohtani T, Khan 0, et al. Epigenomic-Guided Mass Cytometry Profiling Reveals Disease-Specific Features of Exhausted CD8 T Cells. Immunity 2018; 48:1029-1045.e5.
47. Sen D R, Kaminski J, Barnitz R A, et al. The epigenetic landscape of T cell exhaustion. Science 2016; 354:1165-1169.
48. Khan O, Giles J R, McDonald S, et al. TOX transcriptionally and epigenetically programs CD8+ T cell exhaustion. Nature 2019; 571:211-218.
49. Di Pilato M, Kfuri-Rubens R, Pruessmann J N, et al. CXCR6 positions cytotoxic T cells to receive critical survival signals in the tumor microenvironment. Cell 2021: S0092-8674(21)00856-4.
50. Guo X, Zhang Y, Zheng L, et al. Global characterization of T cells in non-small-cell lung cancer by single-cell sequencing. Nat Med 2018; 24:978-985.
51. Zheng C, Zheng L, Yoo J-K, et al. Landscape of Infiltrating T Cells in Liver Cancer Revealed by Single-Cell Sequencing. Cell 2017; 169:1342-1356.e16.
52. Chung W, Eum H H, Lee H-0, et al. Single-cell RNA-seq enables comprehensive tumour and immune cell profiling in primary breast cancer. Nat Commun 2017; 8:15081.
53. Davidson T B, Lee A, Hsu M, et al. Expression of PD-1 by T Cells in Malignant Glioma Patients Reflects Exhaustion and Activation. Clin Cancer Res 2019;
54. Ruperez C, Ferrer-Curriu G, Cervera-Barea A, et al. Meteorin-like/Meteorin-β protects heart against cardiac dysfunction. J Exp Med 2021; 218:e20201206.
55. Leone R D, Zhao L, Englert J M, et al. Glutamine blockade induces divergent metabolic programs to overcome tumor immune evasion. Science 2019; 366:1013-1021.
56. Bader J E, Voss K, Rathmell J C. Targeting Metabolism to Improve the Tumor Microenvironment for Cancer Immunotherapy. Mol Cell 2020; 78:1019-1033.
57. Yu Y-R, Imrichova H, Wang H, et al. Disturbed mitochondrial dynamics in CD8+ TILs reinforce T cell exhaustion. Nat Immunol 2020; 21:1540-1551.
58. Vardhana S A, Hwee M A, Berisa M, et al. Impaired mitochondrial oxidative phosphorylation limits the self-renewal of T cells exposed to persistent antigen. Nat Immunol 2020; 21:1022-1033.
59. Chen Z, Ji Z, Ngiow S F, et al. TCF-1-Centered Transcriptional Network Drives an Effector versus Exhausted CD8 T Cell-Fate Decision. Immunity 2019; 51:840-855.e5.
60. Siddiqui I, Schaeuble K, Chennupati V, et al. Intratumoral Tcf1+PD-1+CD8+ T Cells with Stem-like Properties Promote Tumor Control in Response to Vaccination and Checkpoint Blockade Immunotherapy. Immunity 2019; 50:195-211.e10.
61. Miller B C, Sen D R, Al Abosy R, et al. Subsets of exhausted CD8+ T cells differentially mediate tumor control and respond to checkpoint blockade. Nat Immunol 2019;
62. Ritchie M E, Phipson B, Wu D, et al. limma powers differential expression analyses for RNA-sequencing and microarray studies. Nucleic Acids Res 2015; 43:e47.
63. Li B, Dewey C N. RSEM: accurate transcript quantification from RNA-Seq data with or without a reference genome. BMC Bioinformatics 2011; 12:323.
64. Sfanos K S, Bruno T C, Meeker A K, De Marzo A M, Isaacs W B, Drake C G. Human prostateinfiltrating CD8+T lymphocytes are oligoclonal and PD-1+. Prostate 2009; 69:1694-1703.
65. Subramanian A, Tamayo P, Mootha V K, et al. Gene set enrichment analysis: a knowledgebased approach for interpreting genome-wide expression profiles. Proc Natl Acad Sci USA 2005; 102:15545-15550.
66. Zeng J, See A P, Phallen J, et al. Anti-PD-1 blockade and stereotactic radiation produce longterm survival in mice with intracranial gliomas. Int J Radiat Oncol Biol Phys 2013; 86:343-349.
67. Odorizzi P M, Pauken K E, Paley M A, Sharpe A, Wherry E J. Genetic absence of PD-1 promotes accumulation of terminally differentiated exhausted CD8+ T cells. J Exp Med 2015; 212:1125-1137.

### Example 2: T-cells That Cannot Produce METRNL Have Better Anti-Tumor Activity

The present inventors' data demonstrate for the first time that Meteorin-like (METRNL) is an immunosuppressive cytokine secreted by tumor-infiltrating T cells. Based on these data, the present inventors hypothesize that a T cell that cannot produce Metml upon entering the tumor microenvironment will have better antitumor activity and, therefore, prove superior for immunotherapy.

In order to evaluate this clinical application, METRNL^{-/-}OT-1 mice are produced. OT-1 mice are genetically modified so that their CD8 T cells express a T cell receptor (TCR) specific for SIINFEKL (a peptide derived from ovalbumin). OT-1 mice are bred with METRNL KO mice to develop a line of OT-1/METRNL^{-/-} mice. CD8 T cells are isolated from these animals and transferred into wild-type mice bearing tumors expressing ovalbumin. As a control, OT-1 cells capable of producing Metml are transferred to tumor bearing animals. The present inventors expect that OT-1/METRNL WT cells will demonstrate some antitumor activity and tumors will grow more slowly in these animals than untreated animals while OT-1/METRNL^{-/-} cells will have superior efficacy to METRNL WT cells. If this hypothesis is correct, METRNL could be knocked out in any CAR T cells used as a human therapeutic with the expectation that this would improve performance of the CAR T cell product.

It is plausible that CD8 T cells lacking the METRNL receptor will also exhibit superior antitumor activity. The METRNL receptor is currently unknown. To identify this receptor/receptor complex, co-immunoprecipitation is performed. Once this receptor is identified, a mouse lacking this receptor is generated using CRISPR technology. These mice are then crossed with OT-1 mice and adoptive transfer experiments are carried out in preclinical tumor models as described above.

### Example 3: Treatment of Inflammatory Disorders

The present inventors have shown that Metrnl protein profoundly suppresses T cell responses in vitro in a dose-dependent manner. Accordingly, the present inventors hypothesize that Metrnl protein or another agonist of the Metrnl pathway may be used to treat inflammatory disorders. The hypothesis is tested by administering METRNL in EAE models and other models of autoimmunity.

### Example 4: CXCR6 is an Immunosuppressive Cytokine in CD8 Tumor-Infiltrating Lymphocytes

As described herein, the present inventors performed two different analyses to identify novel genes associated with an exhausted phenotype in CD8 TIL. First, a differential expression analysis was performed for each subgroup against all other CD8 samples (e.g. PD-1+, LAG-3+, TIM-3+ against all other samples, PD-1+, LAG-3+, TIM-3− against all other samples, etc.). The present inventors were particularly interested in the contrast between triple positive (PD-1+, LAG-3+, TIM-3+) TILs against all other samples. The results of these differential expression analyses are provided as a sortable spreadsheet (not shown). The false discovery rate adjustment of p-values was carried out globally for all contrasts. This analysis identified 13 genes differentially expressed by triple positive cells compared with all other samples with an FDR threshold of 0.01 (FIG. 2A).

To isolate gene expression patterns associated with intratumoral location, GSEA analysis of checkpoint triple positive CD8 TILs vs. triple positive PBL was performed. Comparing this analysis with the GSEA analysis of CD8 TILs vs. all PBL revealed that intratumoral location was associated with distinct patterns of differential gene expression. Therefore, a separate differential expression analysis was performed to identify genes associated with intratumoral location among checkpoint-expressing CD8 T cells. For each tumor type, triple positive TILs were contrasted against triple positive activated PBL samples, selecting patients in common for the two sample groups. The results of this analysis identified 36 differentially expressed genes (FDR<0.0001) (FIG. 2B). Combing the results of these two analyses identified 2 genes represented on both lists, CXCR6 and METRNL.

CD8 T cell exhaustion limits immunity against solid tumors as density and quality of CD8 TILs correlate with clinical outcomes. Immune checkpoints are markers of T cell exhaustion and have been targeted with blocking antibodies; however, CIs fail in most patients. One hypothesis for CI failure is that TILs enter a state of severe, irreversible exhaustion characterized by specific genetic and epigenetic programs. In this study, the present inventors used a comparative transcriptomics approach to identify differential gene expression patterns in sorted CD8 TILs and matched PBLs from patients with GBM, prostate cancer, RCC, and bladder cancer to identify differentially regulated genes associated with intratumoral location and exhaustion makers across tumor types. This screen identified METRNL and CXCR6 as candidate targets. Of note, CXCR6 was recently validated in a separate study as the most highly expressed chemokine receptor in TILs, and was reported to be critical for cytotoxic cell-mediated tumor control. These results further support the validity of the screen. Here, the present inventors report that CXCR6 functions as a reversible metabolic checkpoint in TILs.

Single-cell RNA sequencing studies have highlighted TIL heterogeneity in several tumor types. To identify common genetic threads, the present inventors reasoned that CD8 TIL-specific genes could be identified from bulk RNA sequencing data of sorted CD8 T cells by comparing differential gene expression of CD8 TILs and PBLs. To discover novel genes associated with an exhausted immunophenotype, it was further hypothesized that transcript levels of PD-1, LAG-3, and TIM-3 in each sample could be leveraged to infer relative degrees of exhaustion among samples. Based on this assumption, the present inventors developed a model to classify samples as “positive (+)” or “negative (−)” for checkpoint molecules using an expectation maximization algorithm. As expected, CD8 TIL samples were typically positive for multiple immune checkpoints, while naive PBL samples were negative. It is also notable that GBM TILs had the highest overall expression of one or more checkpoints, consistent with previous reports.

The present inventors employed the model to identify novel targets co-expressed with immune checkpoints across tumor types. This analysis identified 13 genes of interest. These genes have diverse functions, including cell migration/adhesion (THBS1, CCR1, CXCR6), apoptosis (DAPK2), lysosomal function (CTSD), and metabolism (METRNL, EPAS1, PFKFB 3). The present inventors sought to further refine the screen by identifying genes correlated with intratumoral location. This analysis yielded 36 differentially expressed genes. CXCR6 and METRNL was present on both lists, so the present inventors hypothesized that CXCR6 and METRNL are specific TIL exhaustion markers based on co-expression with immune checkpoints and independent association with intratumoral location.

### Example 5: T-Cells that Cannot Produce CXCR6 have Better Anti-Tumor Activity

The present inventors' data demonstrate for the first time that CXCR6 is an immunosuppressive cytokine secreted by tumor-infiltrating T-cells. Based on these data, the present inventors hypothesize that a T-cell that cannot produce CXCR6 upon entering the tumor microenvironment will have better antitumor activity and, therefore, prove superior for immunotherapy.

In order to evaluate this clinical application, CXCR6^{-/-} OT-1 mice are produced. OT-1 mice are genetically modified so that their CD8 T-cells express a T-cell receptor (TCR) specific for SIINFEKL (a peptide derived from ovalbumin). OT-1 mice are bred with CXCR6 KO mice to develop a line of OT-1/CXCR6^{-/-} mice. CD8 T-cells are isolated from these animals and transferred into wild-type mice bearing tumors expressing ovalbumin. As a control, OT-1 cells capable of producing CXCR6 are transferred to tumor bearing animals. The present inventors expect that OT-1/CXCR6 WT-cells will demonstrate some antitumor activity and tumors will grow more slowly in these animals than untreated animals while OT-1/CXCR6^{-/-} cells will have superior efficacy to CXCR6 WT-cells. If this hypothesis is correct, CXCR6 could be knocked out in any CAR T-cells used as a human therapeutic with the expectation that this would improve performance of the CAR T-cell product.

### Example 7: T-cells That Cannot Produce CXCL16 Are Expected to Have Better Anti-Tumor Activity

It is plausible that CD8 T-cells lacking the CXCR6 ligand—CXCL16—will also exhibit superior antitumor activity. A mouse lacking CXCL16 is generated using CRISPR technology. These mice are then crossed with OT-1 mice and adoptive transfer experiments are carried out in preclinical tumor models as described above.

### Example 8: Treatment of Inflammatory Disorders

The present inventors have shown that METRNL protein profoundly suppresses T-cell responses in vitro in a dose-dependent manner. Accordingly, along these same lines, the present inventors hypothesize that CXCL16 protein or another agonist of the CXCL16 pathway may be used to treat inflammatory disorders. The hypothesis is tested by administering CXCL16 in EAE models and other models of autoimmunity.

That which is claimed:

1. An engineered T-cell comprising disruption in an endogenous Meteorin-Like (METRNL) gene sequence.

2. An engineered T-cell comprising (a) at least one chimeric antigen receptor (CAR); and (b) at least one genomic disruption of METRNL.

3. The engineered T-cell of claim 2, wherein the genomic disruption is performed using a CRSIPR endonuclease system.

4. A method of treating cancer in a patient comprising the step of administering to the patient an effective amount of the engineered T cell of any of claims 1-3.

5. A method for treating cancer in a patient comprising the step of administering to the patient an effective amount of a METRNL inhibitor.

6. The method of claim 4, wherein the inhibitor is a small molecule, antibody or an inhibitory nucleic acid molecule.

7. The method of claim 5, wherein the inhibitory nucleic acid molecule is an siRNA, shRNA, antisense RNA or ribozyme.

8. A method for treating an autoimmune disorder in a patient comprising the step of administering to the patient an effective amount of a METRNL agonist.

9. An engineered T-cell comprising a disruption in an endogenous C-X-C Motif Chemokine Receptor 6 (CXCR6) gene sequence.

10. An engineered T-cell comprising (a) at least one chimeric antigen receptor (CAR); and (b) at least one genomic disruption of CXCR6.

11. The engineered T-cell of claim 10, wherein the genomic disruption is performed using a CRSIPR endonuclease system.

12. A method of treating cancer in a patient comprising the step of administering to the patient an effective amount of the engineered T-cell of any of claims 9-11.

13. A method for treating cancer in a patient comprising the step of administering to the patient an effective amount of a CXCR6 inhibitor.

14. The method of claim 13, wherein the inhibitor is a small molecule, antibody or an inhibitory nucleic acid molecule.

15. The method of claim 14, wherein the inhibitory nucleic acid molecule is an siRNA, shRNA, antisense RNA or ribozyme.

16. A method for treating an autoimmune disorder in a patient comprising the step of administering to the patient an effective amount of a CXCR6 agonist.

17. An engineered T-cell comprising a disruption in an endogenous C-X-C Motif Ligand 16 (CXCL16) gene sequence.

18. An engineered T-cell comprising (a) at least one chimeric antigen receptor (CAR); and (b) at least one genomic disruption of CXCL16.

19. The engineered T-cell of claim 18, wherein the genomic disruption is performed using a CRSIPR endonuclease system.

20. A method of treating cancer in a patient comprising the step of administering to the patient an effective amount of the engineered T-cell of any of claims 17-19.

21. A method for treating cancer in a patient comprising the step of administering to the patient an effective amount of a CXCL16 inhibitor.

22. The method of claim 21, wherein the inhibitor is a small molecule, antibody or an inhibitory nucleic acid molecule.

23. The method of claim 2, wherein the inhibitory nucleic acid molecule is an siRNA, shRNA, antisense RNA or ribozyme.

24. A method for treating an autoimmune disorder in a patient comprising the step of administering to the patient an effective amount of a CXCL16 agonist.
